(19) United States
(12) Patent Application Publication
(10) Pub. No.: US 2024/0360472 A1
(43) Pub. Date: Oct. 31, 2024

(54) POLYNUCLEOTIDES AND USES THEREOF

(71) Applicant: STRAND THERAPEUTICS INC., Cambridge, MA (US)

(72) Inventors: Jaspreet S. KHURANA, Boston, MA (US); Aalok SHAH, Shrewsbury, MA (US); Tasuku KITADA, Boston, MA (US)

(73) Assignee: STRAND THERAPEUTICS INC., Boston, MA (US)

(21) Appl. No.: 18/294,799

(22) PCT Filed: Aug. 3, 2022
(86) PCT No.: PCT/US2022/074491

§ 371 Date: Feb. 2, 2024

Related U.S. Application Data
(60) Provisional application No. 63/228,892, filed on Aug. 3, 2021.

(51) Int. Cl. C12N 15/86; A61K 38/00; C07K 14/005; C07K 14/54

(52) U.S. Cl. CPC C12N 15/86; C07K 14/005; C07K 14/5434; A61K 38/00; C12N 2760/16122; C12N 2770/36143

(57) ABSTRACT

The present disclosure relates to a polynucleotide or a set of polynucleotides comprising a first nucleic acid molecule encoding an innate immune inhibitor, e.g., an influenza non-structural (NS1) protein, and a second nucleic acid molecule encoding a heterologous target mRNA. The disclosure also includes methods of making the polynucleotides, methods of modifying a cell, and methods of treating a subject using the same.

### CROSS-REFERENCE TO RELATED APPLICATION

This PCT application claims the priority benefit of U.S. Provisional Application No. 63/228,892, filed Aug. 3, 2021, which is incorporated herein by reference in its entirety.

### REFERENCE TO SEQUENCE LISTING SUBMITTED ELECTRONICALLY VIA EFS WEB

The content of the electronically submitted sequence listing (4597_008PC01_Seqlisting_st26.xml; Size: 96,785 bytes; and Date of Creation: Aug. 3, 2022) submitted in this application is incorporated herein by reference in its entirety.

### FIELD OF THE DISCLOSURE

The present disclosure provides isolated polynucleotides (e.g., replicons) or a set of polynucleotides that comprise a first nucleotide sequence encoding innate immune inhibitor, e.g., an influenza non-structural (NS1) protein, and a second nucleotide sequence encoding a heterologous target mRNA. Such polynucleotides are capable of driving enhanced and persistent expression of the heterologous target mRNA in a cell.

### BACKGROUND OF THE DISCLOSURE

Nucleic acid therapeutics have emerged as a promising and rapidly developing treatment for a wide variety of diseases. These therapies rely on cells, in vitro, ex vivo, or in vivo, to produce biologically active molecules, such as functional RNAs and/or therapeutic polypeptides, in a way that retains native conformations and post-translational modifications, which are often difficult to achieve with recombinant proteins. Synthetic mRNA has proven to be a valuable tool, with an improved safety profile relative to viral or DNA-based modalities. However, the human immune system naturally degrades RNA, limiting the potency and persistence of administered synthetic RNAs (e.g., circular RNAs). As such, there remains a need in the art for RNA therapies that provide potent and durable effects in vitro and in vivo.

### BRIEF SUMMARY OF THE DISCLOSURE

Some aspects of the present disclosure are directed to a polynucleotide or a set of polynucleotides comprising a first nucleic acid molecule encoding an influenza non-structural (NS1) protein and a second nucleic acid molecule encoding a heterologous target mRNA.

In some aspects, the first nucleic acid molecule encoding the influenza NS1 protein and the second nucleic acid molecule encoding the target mRNA are present in a first vector. In some aspects, the first nucleic acid molecule encoding the influenza NS1 protein is present in a first vector, and wherein the second nucleic acid molecule encoding the target mRNA is present in a second vector.

In some aspects, the first nucleic acid molecule encoding the influenza NS1 protein is expressed under the control of a first promoter. In some aspects, the second nucleic acid molecule encoding the target mRNA is expressed under the control of a second promoter. In some aspects, the first promoter and the second promoter are the same. In some aspects, the first promoter and the second promoter are the different.

In some aspects, the first nucleic acid molecule encoding the influenza NS1 protein and the second nucleic acid molecule encoding the target mRNA are expressed under the control of a first promoter, wherein the first promoter drives expression of both the influenza NS1 protein and the target mRNA. In some aspects, the first nucleic acid molecule encoding the influenza NS1 protein and the second nucleic acid molecule encoding the target mRNA are linked by an IRES sequence. In some aspects, the first vector, the second vector, or both comprise one or more regulatory elements.

In some aspects, the expression of the target mRNA is increased relative to the expression of the target mRNA in the absence of the first nucleic acid molecule encoding the influenza NS1 protein. In some aspects, the expression of the target mRNA is increased by at least about 10%, at least about 20%, at least about 25%, at least about 30%, at least about 40%, at least about 50%, at least about 60%, at least about 70%, at least about 75%, at least about 80%, at least about 90%, at least about 100%, at least about 125%, at least about 150%, at least about 175%, at least about 200%, at least about 225%, at least about 250%, at least about 275%, or at least about 300% relative to the expression of the target mRNA in the absence of the first nucleic acid molecule encoding the influenza NS1 protein. In some aspects, the increase in the expression of the target mRNA persists for at least about 6 hours, at least about 12 hours, at least about 18 hours, at least about 24 hours, at least about 30 hours, at least about 36 hours, at least about 42 hours, or at least about 48 hours.

In some aspects, the target mRNA encodes a biologically active polypeptide. In some aspects, the biologically active polypeptide comprises a cytokine, a chemokine, a growth factor, a clotting factor, an enzyme, or any combination thereof. In some aspects, the cytokine is IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP, a fragment thereof, a variant thereof, or any combination thereof.

In some aspects, the target mRNA encodes an IL-12 polypeptide or a fragment or variant thereof. In some aspects, the target mRNA encodes a p35 subunit of IL-12 and a p40 subunit of IL-12. In some aspects, the p35 subunit and the p40 subunit are expressed from a single promoter. In some aspects, the p35 subunit and the p40 subunit are expressed as a single contiguous polypeptide. In some aspects, the p35 subunit and the p40 subunit are linked by one or more covalent bonds. In some aspects, the p35 subunit and the p40 subunit are linked by one or more peptide bonds. In some aspects, a portion of the mRNA that encodes the p35 subunit is separated from a portion of the mRNA that encodes the p40 subunit by an IRES.

In some aspects, the target mRNA encodes a miRNA, siRNA, shRNA, a dsRNA, antisense oligonucleotide, a guide RNA, or any combination thereof.

In some aspects, the first promoter is an inducible promoter, a tissue specific promoter, or a constitutively active promoter. In some aspects, the second promoter is an inducible promoter, a tissue specific promoter, or a constitutively active promoter.

In some aspects, the influenza NS1 is a type A influenza virus NS1, a type B influenza virus NS1, a type C influenza virus NS1, or a variant thereof. In some aspects, the influenza NS1 is an H1N1 NS1, H1N2 NS1, H2N2 NS1, H3N2 NS1, H5N1 NS1, H7N9 NS1, H7N7 NS1, H9N2 NS1, H7N2 NS1, H7N3 NS1, H5N2 NS1, H10N7 NS1, or any combination thereof. In some aspects, the influenza NS1 is H5N1 NS1. In some aspects, the influenza NS1 is H1N1 NS1. In some aspects, the H1N1 NS1 is the H1N1 TX91 variant NS1.

In some aspects, the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 1 or 2. In some aspects, the influenza NS1 encoded by the first nucleic acid molecule comprises the amino acid sequence encoded by the nucleotide sequence set forth in SEQ ID NO: 1 or 2. In some aspects, the first nucleic acid molecule comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1 or 2, wherein the nucleotide sequence encodes an influenza NS1 protein. In some aspects, the first nucleic acid molecule comprises the nucleotide sequence set forth in SEQ ID NO: 1 or 2, wherein the nucleotide sequence encodes an influenza NS1 protein. In some aspects, (i) the first nucleic acid molecule, (ii) the second nucleic acid molecule, or (iii) both (i) and (ii) are circular RNA

In some aspects, the polynucleotide or the set of polynucleotides comprises one or more modified nucleic acid molecule.

Some aspects of the present disclosure are directed to a polynucleotide or a set of polynucleotides comprising a self-replicating target mRNA, wherein the self-replicating target mRNA comprises one or more modified nucleic acid molecule. In some aspects, less than about 50%, less than about 45%, less than about 40%, less than about 35%, less than about 30%, or less than about 25% of the nucleic acids in the polynucleotide or the set of polynucleotides are modified nucleic acid molecules. In some aspects, about 25% of the nucleic acids in the polynucleotide or the set of polynucleotides are modified nucleic acid molecules. In some aspects, the one or more modified nucleic acid molecule is a modified rNTP. In some aspects, the one or more modified nucleic acid molecule comprises N1-methylpsuedo uracil, 5-methyl cytosine, N6-methyladenosine or combinations thereof.

Some aspects of the present disclosure are directed to a vector or a set of vectors comprising a polynucleotide or a set of polynucleotides disclosed herein. In some aspects, the vector is a replicon. In some aspects, the vector is a Venezuelan equine encephalitis (VEE) replicon or a derivative or portion thereof. In some aspects, the vector is a Venezuelan equine encephalitis (VEE) replicon comprising a nucleotide sequence encoding a lysine at residue 739, according to the wild-type amino acid sequence VEE.

In some aspects, the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA; and (vi) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA; (v) an E1 sequence; and (vi) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA; (vi) an E1 sequence; and (vii) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA, wherein the target mRNA encodes a human IL-12 polypeptide; (vi) an E1 sequence; and (vii) a VEE 3′UTR or a derivative thereof.

T In some aspects, the vector comprises (i) a VEE 5′UTR or a derivative thereof, (ii) one or more non-structural protein (nsP); (iii) a first nucleic acid molecule encoding an H1N1 TX91 variant NS1 or an H5N1 NS1; (iv) a second nucleic acid molecule encoding the target mRNA; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a P2A linker; (v) a second nucleic acid molecule encoding the target mRNA; (vi) an E1 sequence; and (vii) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a P2A linker; (v) a second nucleic acid molecule encoding the target mRNA, wherein the target mRNA encodes a human IL-12 polypeptide; (vi) an E1 sequence; and (vii) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding an H1N1 TX91 variant NS1; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule comprising the nucleotide sequence set forth in SEQ ID NO: 1; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule encoding an H5N1 NS1; (iv) a P2A linker; (v) a second nucleic acid molecule encoding the target mRNA, wherein the target mRNA encodes a human IL-12 polypeptide; (vi) an E1 sequence; and (vii) a VEE 3′UTR or a derivative thereof.

In some aspects, the vector comprises (i) a VEE 5′UTR or a derivative thereof; (ii) one or more non-structural protein (nsP); (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 2; (iv) a second nucleic acid molecule encoding the target mRNA, wherein the target mRNA encodes a human IL-12 polypeptide; and (v) a VEE 3′UTR or a derivative thereof.

In some aspects, the vector comprises (i) a VEE 5′UTR or a derivative thereof; (ii) one or more non-structural protein (nsP); (iii) a first nucleic acid molecule comprising the nucleotide sequence set forth in SEQ ID NO: 2; (iv) a second nucleic acid molecule encoding the target mRNA, wherein the target mRNA encodes a human IL-12 polypeptide; and (v) a 3′UTR from a parent replicon, e.g., a 3′UTR from VEE. In some aspects, the one or more nsP comprises a VEE nsP or a derivative thereof. In some aspects, the VEE nsP is selected from nsP2, nsP3, nsP4, and any combination thereof.

Some aspects of the present disclosure are directed to a cell comprising a polynucleotide or a set of polynucleotides disclosed here or a vector or a set of vectors disclosed herein. In some aspects, the cell is a mammalian cell. In some aspects, the cell is a human cell. In some aspects, the cell is an immune cell.

Some aspects of the present disclosure are directed to a pharmaceutical composition comprising a polynucleotide or a set of polynucleotides disclosed herein, a vector or a set of vectors disclosed herein, or a cell disclosed herein and a pharmaceutically acceptable carrier.

Some aspects of the present disclosure are directed to a method of expressing a target mRNA in a cell, comprising transfecting the cell with a polynucleotide or a set of polynucleotides disclosed herein or a vector or a set of vectors disclosed herein. In some aspects, the cell is a human cell. In some aspects, the cell is an ex vivo human cell. In some aspects, the cell is a human immune cell.

Some aspects of the present disclosure are directed to a method of treating a subject in need thereof, comprising administering to the subject a polynucleotide or a set of polynucleotides disclosed herein, a vector or a set of vectors disclosed herein, a cell disclosed herein, or a pharmaceutical composition disclosed herein.

Some aspects of the present disclosure are directed to a method of expressing a target mRNA in a subject in need thereof, comprising administering to the subject a polynucleotide or a set of polynucleotides disclosed herein, a vector or a set of vectors disclosed herein, a cell disclosed herein, or a pharmaceutical composition disclosed herein.

In some aspects, the subject is afflicted with a cancer. In some aspects, the cancer is selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, Hodgkin's lymphoma, and any combination thereof.

Some aspects of the present disclosure are directed to a method of expressing a target mRNA in a cell, comprising co-expressing the target mRNA and an influenza NS1 protein in the cell, wherein the target mRNA is not an influenza mRNA. In some aspects, the influenza NS1 protein is encoded by a first nucleic acid molecule and the target mRNA is encoded by a second nucleic acid molecule.

In some aspects, the first nucleic acid molecule encoding the influenza NS1 protein and the second nucleic acid molecule encoding the target mRNA are present in a first vector. In some aspects, the first nucleic acid molecule encoding the influenza NS1 protein is present in a first vector, and wherein the second nucleic acid molecule encoding the target mRNA is present in a second vector.

In some aspects, the first nucleic acid molecule encoding the influenza NS1 protein is expressed under the control of a first promoter. In some aspects, the second nucleic acid molecule encoding the target mRNA is expressed under the control of a second promoter. In some aspects, the first promoter and the second promoter are the same. In some aspects, the first promoter and the second promoter are the different. In some aspects, the first nucleic acid molecule encoding the influenza NS1 protein and the second nucleic acid molecule encoding the target mRNA are expressed under the control of a first promoter, wherein the first promoter drives expression of both the influenza NS1 protein and the target mRNA. In some aspects, the first nucleic acid molecule encoding the influenza NS1 protein and the second nucleic acid molecule encoding the target mRNA are linked by an IRES sequence. In some aspects, the first vector, the second vector, or both comprise one or more regulatory elements. In some aspects, expression of the target mRNA is increased relative to the expression of the target mRNA in the absence of the first nucleic acid molecule encoding the influenza NS1 protein. In some aspects, (i) the first nucleic acid molecule, (ii) the second nucleic acid molecule, or (iii) both (i) and (ii) are circular RNA.

Some aspects, of the present disclosure are directed to a method of expressing a target mRNA in a cell, comprising transfecting the cell with a polynucleotide or a set of polynucleotides comprising a self-replicating target mRNA comprising one or more modified nucleic acid molecules. In some aspects, less than about 50%, less than about 45%, less than about 40%, less than about 35%, less than about 30%, or less than about 25% of the nucleic acids in the polynucleotide or the set of polynucleotides are modified nucleic acid molecules. In some aspects, about 25% of the nucleic acids in the polynucleotide or the set of polynucleotides are modified nucleic acid molecules. In some aspects, the one or more modified nucleic acid molecules is a modified rNTP. In some aspects, the one or more modified nucleic acid molecules comprises N1-methylpsuedo uracil, 5-methyl cytosine, N6-methyladenosine, or combinations thereof.

In some aspects, expression of the target mRNA is increased relative to the expression of the target mRNA from a self-replicating target mRNA not comprising one or more modified nucleic acid molecules. In some aspects, the expression of the target mRNA is increased by at least about 10%, at least about 20%, at least about 25%, at least about 30%, at least about 40%, at least about 50%, at least about 60%, at least about 70%, at least about 75%, at least about 80%, at least about 90%, at least about 100%, at least about 125%, at least about 150%, at least about 175%, at least about 200%, at least about 225%, at least about 250%, at least about 275%, or at least about 300%.

In some aspects, the increase in the expression of the target mRNA persists for at least about 6 hours, at least about 12 hours, at least about 18 hours, at least about 24 hours, at least about 30 hours, at least about 36 hours, at least about 42 hours, or at least about 48 hours.

In some aspects, the target mRNA encodes a biologically active polypeptide. In some aspects, the biologically active polypeptide comprises a cytokine, a chemokine, a growth factor, a clotting factor, an enzyme, or any combination thereof. In some aspects, the cytokine is IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP, a fragment thereof, a variant thereof, or any combination thereof.

In some aspects, the target mRNA encodes an IL-12 polypeptide or a fragment or variant thereof. In some aspects, the target mRNA encodes a p35 subunit of IL-12 and a p40 subunit of IL-12. In some aspects, the p35 subunit and the p40 subunit are expressed from a single promoter. In some aspects, the p35 subunit and the p40 subunit are expressed as a single contiguous polypeptide. In some aspects, the p35 subunit and the p40 subunit are linked by one or more covalent bonds. In some aspects, the p35 subunit and the p40 subunit are linked by one or more peptide bonds. In some aspects, a portion of the mRNA that encodes the p35 subunit is separated from a portion of the mRNA that encodes the p40 subunit by an IRES.

In some aspects, the target mRNA comprises a miRNA, a siRNA, a shRNA, a dsRNA, an antisense oligonucleotide, a guide RNA, a circular RNA, or any combination thereof.

In some aspects, the first promoter is an inducible promoter, a tissue specific promoter, or a constitutively active promoter. In some aspects, the second promoter is an inducible promoter, a tissue specific promoter, or a constitutively active promoter.

In some aspects, the influenza NS1 is a type A influenza virus NS1, a type B influenza virus NS1, a type C influenza virus NS1, or a variant thereof. In some aspects, the influenza NS1 is an H1N1 NS1, H1N2 NS1, H2N2 NS1, H3N2 NS1, H5N1 NS1, H7N9 NS1, H7N7 NS1, H9N2 NS1, H7N2 NS1, H7N3 NS1, H5N2 NS1, H10N7 NS1, or a combination thereof. In some aspects, the influenza NS1 is H5N1 NS1. In some aspects, the influenza NS1 is H1N1 NS1. In some aspects, the H1N1 NS1 is the H1N1 TX91 variant NS1.

In some aspects, the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 1 or 2. In some aspects, the influenza NS1 encoded by the first nucleic acid molecule comprises the amino acid sequence encoded by the nucleotide sequence set forth in SEQ ID NO: 1 or 2. In some aspects, the first nucleic acid molecule comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1 or 2, wherein the nucleotide sequence encodes an influenza NS1 protein. In some aspects, the first nucleic acid molecule comprises the nucleotide sequence set forth in SEQ ID NO: 1 or 2, wherein the nucleotide sequence encodes an influenza NS1 protein.

### BRIEF DESCRIPTION OF THE DRAWINGS/FIGURES

FIGS. 1A-1B are illustrations of a sample non-cytopathic-EGFP vector (FIG. 1A) and a non-cytopathic-NS1-EGFP vector (FIG. 1).

FIGS. 2A-2D are images of 4T1 cells 24-hour post-transfection with Strand-mCherry (FIG. 2A), mCherry modRNA (FIG. 2B), and Strand-mCherry plus NS1 modRNA mRNAs (FIGS. 2C-2D) using Lipofectamine MessengerMax.

FIG. 2E summarizes the mean fluorescence intensity observed for each mRNA transfection tested in FIGS. 2A-2D.

FIGS. 3A-3D are images of 4T1 cells 24-hour post-transfection with Strand-mCherry (FIG. 3A), mCherry modRNA (FIG. 3B), and Strand-mCherry plus NS1 modRNA mRNAs (FIGS. 3C-3D) using TT3 lipid nanoparticle.

FIGS. 3E-3F summarize the mean fluorescence intensity observed for each mRNA transfected by tandem NS1 expression (FIG. 3E) or NS1 cotransfection (FIG. 3F).

FIGS. 4A-4H are graphical representations of flow cytometry data illustrating the number of cells expressing mCherry at 24 hours (FIGS. 4A-4D) and 48 hours (FIGS. 4E-4H) after transfection of Hcc38 tumor cells with NS1 modRNA (FIGS. 4B and 4F), NS1 repRNA (FIGS. 4C and 4G), or NS1-P2A-mCherry mRNA (FIGS. 4D and 4H) using Lipofectamine MessengerMax.

FIGS. 4I-4K show median fluorescence intensity (MFI) of Scc9 HNSCC cells (FIG. 41) and FaDu HNSCC cells (FIGS. 4J-4K).

FIGS. 5A-5D are graphical representations of flow cytometry data illustrating the number of cells expressing EGFP at 24 hours following transfection of BT20 cancer using TT3 LNP comprising an EGFP-encoding replicon vector containing either the original (Strand) backbone (FIGS. 5A-5B) or one containing a Q739L mutation (non-cytopathic; FIGS. 5C-5D).

FIGS. 5E-5F are graphical representations of median fluorescence intensity (MFI;

FIG. 5E) and transfection efficiency as measured by the percent of GFP-positive cells (FIG. 5F).

FIGS. 6A-6F are graphical representations of flow cytometry data illustrating the number of B16.F10 cells (FIGS. 6A-6C) and 4T1 cells (FIGS. 6D-6F) expressing mCherry following electroporation with replicon made either using unmodified rNTPs (Unmodified Rep;

FIGS. 6A and 6D) or using a ratio of 1:1 (50%; FIGS. 6B and 6E) or 1:3 (25%; FIGS. 6C and 6F) UTP to N1-methyl-pseudoUTP (denoted by psi).

FIG. 6G is a graphical representation of median fluorescence intensity (MFI) of cells described in FIGS. 6A-6G.

FIG. 7 is a bar graph illustrating the relative luminescence of 4T1 cells transfected with firefly luciferase (Fluc)-encoded replicons made either using unmodified rNTPs (unmod) or using a 1:3 ratio of certain NTPs-U: Ψ (M1) or U: Ψ; 1:3 C:Sme-C(M2), where C refers to Cytidine and 5me-C refers to 5-methyl-cytidine. Luminescence was measured at 24 as 48 hrs post-transfection via electroporation.

FIGS. 8A-8B are bar graphs illustrating the relative luminescence (FIG. 8A) and type I IFN activity as measured using the SEAP reporter assay using colorimetry (FIG. 8B) of B16-ISG cells, an interferon inducible cell line, transfected with firefly luciferase (Fluc)-encoded replicons made either using unmodified rNTPs (unmod) or using a 1:3 ratio of certain NTPs-U: Ψ (M1) or U: Ψ; 1:3 C:5me-C(M2), where C refers to Cytidine and 5me-C refers to 5-methyl-cytidine.

FIGS. 9A-9C are graphical representations of payload expression (FIG. 9A), signal intensity (FIG. 9B), and Type I IFN activity (FIG. 9C) of B16-ISG cells transfected with a Q739L replicon expressing NS1-EGFP (P2A linker) made either using unmodified or singly (M1) or doubly modified rNTPs.

FIGS. 10A-10F are images of GFP expression of T cells activated for 2 days with Anti-CD3/CD28/CD2 cocktail with high dose IL-2 with (FIGS. 10D-10F) or without (FIGS. 10A-10C) addition of a recombinant protein (Enhancer) to the media. T cells were transfected with lipid 1 (FIGS. 10A and 10D), lipid 2-cholesterol (FIGS. 10B and 10E), or lipid 2-β-sitosterol (FIGS. 10C and 10F).

FIGS. 11A-11C are graphical representations of transfection efficiency (percent GFP positive cells; FIG. 11A), median fluorescence intensity (FIG. 11B), and IFN-gamma levels (FIG. 11C) in primary human T cells activated using IL-2 and anti-CD3/CD28/CD2 for 2 days post thaw and transfected using the Q739L replicon driving NS1-EGFP, as used above, and made using unmodified or singly (M1) or doubly modified (M2) mRNAs.

FIGS. 12A-12B are graphical representations of transfection efficiency (percent GFP positive cells; FIG. 12A) and IFN-alpha activation (FIG. 12B) in human PBMCs isolated from three healthy donors and either grown with low dose IL-2 (Resting) or with high dose of IL-2 in presence of Anti-CD3/CD28/CD2 cocktail (Activated) for 2 days followed by mRNA:lipid delivery with M2 modified Q739L replicons driving NS1-EGFP or EGFP or with conventional EGFP mRNA (EGFP-mod). Measurements were taken 24 hours post transfection.

### DETAILED DESCRIPTION OF THE DISCLOSURE

Some aspects of the present disclosure are directed to a polynucleotide or a set of polynucleotides comprising a first nucleic acid molecule encoding an influenza non-structural (NS1) protein and a second nucleic acid molecule encoding a heterologous target mRNA. The present disclosure provides that expression of a target mRNA encoded by a polynucleotide, e.g., a replicon, can be enhanced in the presence of influenza NS1. In particular, coexpression of influenza NS1 and a target mRNA increases the expression level of the target mRNA and the persistence of the expression of the target mRNA in a cell. As such, some aspects of the present disclosure are directed to methods of expressing a target mRNA in a cell, e.g., a human cell, comprising transfecting the cell with a polynucleotide (e.g., circular RNA) or a set of polynucleotides (e.g., set of circular RNAs) comprising a first nucleic acid molecule encoding an influenza non-structural (NS1) protein and a second nucleic acid molecule encoding a heterologous target mRNA. In some aspects, the target mRNA encodes an IL-12 polypeptide or a fragment or variant thereof.

Additional aspects of the present disclosure are provided throughout the present application.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “approximately” or “about,” as applied to one or more values of interest, refers to a value that is similar to a stated reference value and within a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “ug” and “uM“are used interchangeably with”μg” and “μM,” respectively.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither, or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values that are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The terms “administration,” “administering,” and grammatical variants thereof refer to introducing a composition (e.g., such as an isolated polynucleotide described herein) into a subject via a pharmaceutically acceptable route. The introduction of a composition into a subject can be done by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition to perform its intended function. For example, if a suitable route is intravenous, the composition can be administered by introducing the composition into a vein of the subject.

As used herein, the term “cancer” refers to a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream. The compositions disclosed herein can be used in the treatment of any cancer, including but not limited to melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, Hodgkin's lymphoma, and any combination thereof.

The term “coding sequence” or sequence “encoding” is used herein to mean a DNA or RNA region (the transcribed region) which “encodes” a particular protein, e.g., an influenza NS1 protein or a target heterologous protein. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

The term “downstream” refers to a nucleotide sequence that is located 3′ to a reference nucleotide sequence. In some aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound.

The term “expression,” as used herein, refers to a process by which a polynucleotide produces a gene product, e.g., RNA or a polypeptide (e.g., therapeutic protein, e.g., influenza NS1 nonstructural protein). It includes, without limitation, transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and as well as the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA (e.g., circular RNA) or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

As used herein, the term “heterologous target mRNA” refers to any mRNA (linear or circular) that is not naturally present in a target cell that can be expressed in the target cell using the polynucleotides described herein. Unless indicated otherwise, a heterologous target mRNA can encode a polypeptide or RNA molecules that have regulatory function (such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof). Accordingly, as used herein, the term “encode” refers to the production of a moiety of interest (e.g., polypeptide or a RNA molecule, such as circular RNA) from a nucleic acid molecule (e.g., heterologous target mRNA). In some aspects, the heterologous target mRNA encodes a biologically active polypeptide, including but not limited to a cytokine, a chemokine, a growth factor, a clotting factor, an enzyme, or any combination thereof. In some aspects, the heterologous target mRNA is referred to herein as a “payload.” Unless indicated otherwise, the term “target mRNA” and “heterologous target mRNA” are used interchangeably.

In some aspects, two or more sequences are said to be “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “highly conserved” if they are about 70% identical, about 80% identical, about 90% identical, about 95% identical, about 98% identical, or about 99% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

As used herein, the term “identity” refers to the overall monomer conservation between polymeric molecules, e.g., between polypeptide molecules or polynucleotide molecules (e.g., DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage identity (% ID) of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated” and “purified,” and grammatical variants thereof, are used interchangeably and refer to the state of a preparation of desired composition of the present disclosure that has undergone one or more processes of purification. In some aspects, isolating or purifying as used herein is the process of removing, partially removing (e.g., a fraction) of a composition of the present disclosure from a sample containing contaminants. In some aspects, an isolated composition has no detectable undesired activity or, alternatively, the level or amount of the undesired activity is at or below an acceptable level or amount. In some aspects, an isolated composition has an amount and/or concentration of desired composition of the present disclosure, at or above an acceptable amount and/or concentration and/or activity. In some aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.99990%, or greater than 99.9999% as compared to the starting material. In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

As used herein, the term “nonstructural protein” refers to a protein encoded by a virus but that is not part of the viral particle. More specifically, the nonstructural proteins described herein comprise the influenza NS1 protein, including but not limited to a type A influenza virus NS1, a type B influenza virus NS1, a type C influenza virus NS1, an H1N1 NS1, an H1N2 NS1, an H2N2 NS1, an H3N2 NS1, an H5N1 NS1, an H7N9 NS1, an H7N7 NS1, an H9N2 NS1, an H7N2 NS1, an H7N3 NS1, an H5N2 NS1, an H10N7 NS1, combinations thereof, or variants thereof. Additional disclosures relating to such nonstructural proteins are provided elsewhere in the present disclosure.

“Nucleic acid,” “nucleic acid molecule,” “nucleotide sequence,” and grammatical variants thereof, are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form or a double-stranded helix. Additionally, as is apparent from the present disclosure, in some aspects, a nucleic acid molecule can also be in a circular form (e.g., circular RNA). Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA.

As used herein, the term “circular RNA” refers to a polyribonucleotide that forms a circular structure through covalent bonds. As is apparent from the present disclosure, any of the polynucleotides, sets of polynucleotides, first nucleic acid molecule, and second nucleic acid molecule can be circular in structure. For instance, in some aspects, a polynucleotide described herein (e.g., comprising a first nucleic acid molecule encoding an influenza NS1 protein and a second nucleic acid encoding a heterologous target mRNA) comprises a circular RNA. In some aspects, a first nucleic acid molecule provided herein (e.g., encoding an influenza NS1 protein) comprises a circular RNA. In some aspects, a second nucleic acid molecule provided herein (e.g., encoding a heterologous target mRNA) comprises a circular RNA. In some aspects, a polynucleotide provided herein comprises a circular RNA, which comprises the first nucleic acid molecule and the second nucleic acid molecule.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the polynucleotides described herein mixed, or intermingled with, or suspended in one or more other chemical components, such as pharmaceutically-acceptable carriers and excipients. In some aspects, a purpose of a pharmaceutical composition is to facilitate administration of preparations of polynucleotides to a subject.

The term “polynucleotide” as used herein refers to polymers of nucleotides of any length, including ribonucleotides, deoxyribonucleotides, analogs thereof, or mixtures thereof. In some aspects, a polynucleotide useful for the present disclosure can be linear. In some aspects, a polynucleotide is circular (e.g., circular RNA). This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

More particularly, the term “polynucleotide” includes polydeoxyribonucleotides (containing 2-deoxy-D-ribose), polyribonucleotides (containing D-ribose), including tRNA, rRNA, hRNA, siRNA, and mRNA (including circular RNA), whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multi-chain polypeptides. Most commonly, disulfide linkages are found in multi-chain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof, as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof, are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The disclosures provided herein are applicable to both human therapy and veterinary applications.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In some aspects, the term “treating” or “treatment” means inducing an immune response in a subject against an antigen (e.g., heterologous payload disclosed herein).

The term “upstream” refers to a nucleotide sequence that is located 5′ to a reference nucleotide sequence.

### II. Compositions of the Disclosure

Some aspects of the present disclosure are directed to a polynucleotide or a set of polynucleotides comprising a first nucleic acid molecule encoding an innate immune inhibitor and a second nucleic acid molecule encoding a heterologous target mRNA. In some aspects, the first nucleic acid molecule is circular. In some aspects, the second nucleic acid molecule is circular. In some aspects, both the first nucleic acid molecule and the second nucleic acid molecule are circular. As described herein, in some aspects, the polynucleotide or set of polynucleotides comprise circular RNA. Accordingly, in some aspects, provided herein is a circular RNA comprising a first nucleic acid molecule encoding an innate immune inhibitor and a second nucleic acid molecule encoding a heterologous target mRNA. Also provided herein is a set of circular RNAs comprising a first nucleic acid molecule encoding an innate immune inhibitor and a second nucleic acid molecule encoding a heterologous target mRNA.

Any innate immune inhibitor can be used in the compositions and methods disclosed herein. Examples of innate immune inhibitors include, but are not limited to Influenza NS1, African swine fever virus (ASFV) g5R, Coxsackievirus B3 (CVB3) 2A protease, CVB3 3C protease, encephalomyocarditis virus (EMCV) 2A protein (e.g., without NLS), EMCV 3C protease, feline calicivirus (FCV) 3C-like protease, foot-and mouth disease virus (FMDV) L protease, group A rotavirus (RVA) NSP3, hantavirus N, human adenovirus 5 (Ad5) 100K, human immunodeficiency virus 1 (HIV-1) protease, human rhinovirus (HRV) 2A protease, HRV 3C protease, human herpesvirus 1 (HSV) vhs, human herpesvirus 1 (HSV) vhs, human T-cell leukemia virus (HTLV-1) protease, Influenza A virus (FluAv) Pol, human herpesvirus 8 (KSHV) SOX, MD145-12 3C-like protease, measles virus (MV) N, poliovirus (PV) 2A protease, PV 3C protease, moloney murine leukemia virus (MMLV) protease 3C, rabies virus (RV) M, SARS-CoV NSP1, SARS-CoV S, SARS-CoV spike, simian virus 40 (SV40) small T antigen, vaccinia virus (VV) D10, VV D9, mouse 4E-BP1 (e.g., constitutive active), mouse 4E-BP2 (e.g., constitutive active), mouse 4E-BP3 (e.g., constitutive active), mouse 4EHP, mouse Ago1, mouse Ago2, mouse Ago3, mouse Ago4, mouse CPEB2, mouse DDX6, mouse eIF4E, mouse eIF4E (S209A), mouse eIF4E (S209D), mouse eIF4E (S209E), mouse eIF4g (N-term), mouse FMRP, mouse GW182, mouse p54, mouse p56 mouse p60, mouse PABP (eIF4G binding domain), mouse PDCD4, mouse RNase L (NΔ385: constitutive active), mouse Upfl (e.g., constitutive active), mouse (Me31B), EBFP2, any derivative thereof, and any combination thereof. In some aspects, the innate immune inhibitor is an innate immune inhibitor disclosed in US Publication No. 20180296702 A, which is incorporated by reference herein in its entirety. In some aspects, the innate immune inhibitor comprises a viral non-structural (NS) protein, e.g., an influenza NS1 protein. In some aspects, the first nucleic acid molecule encodes a viral non-structural (NS) protein. In some aspects, the first nucleic acid molecule encodes an influenza NS1 protein or a derivative thereof.

In some aspects, the first nucleic acid molecule encoding the innate immune inhibitor, e.g., influenza NS1 protein, and the second nucleic acid molecule encoding the target mRNA are present in a first vector. In some aspects, the first nucleic acid molecule encoding the innate immune inhibitor, e.g., influenza NS1 protein, is present in a first vector, and the second nucleic acid molecule encoding the target mRNA is present in a second vector.

In some aspects, the first nucleic acid molecule encoding the innate immune inhibitor, e.g., influenza NS1 protein, and the second nucleic acid molecule encoding the target mRNA are expressed under the control of a single promoter. In some aspects, the first nucleic acid molecule encoding the innate immune inhibitor, e.g., influenza NS1 protein, and the second nucleic acid molecule encoding the target mRNA are transcribed as a polycistronic mRNA. In some aspects, the first nucleic acid molecule encoding the innate immune inhibitor, e.g., influenza NS1 protein, is expressed under the control of a first promoter, and the second nucleic acid molecule encoding the target mRNA is expressed under the control of a second promoter. In some aspects, the first promoter and the second promoter are the same. In some aspects, the first promoter and the second promoter are different.

In some aspects, the first nucleic acid molecule encoding the innate immune inhibitor, e.g., influenza NS1 protein, and the second nucleic acid molecule encoding the target mRNA are expressed under the control of a first promoter, wherein the first promoter drives expression of both the innate immune inhibitor, e.g., influenza NS1 protein, and the target mRNA. In some aspects, the first nucleic acid molecule encoding the innate immune inhibitor, e.g., influenza NS1 protein, and the second nucleic acid molecule encoding the target mRNA are linked by an IRES sequence.

In some aspects, the first nucleic acid molecule encoding the innate immune inhibitor, e.g., influenza NS1 protein, the second nucleic acid molecule encoding the target mRNA, or both are expressed under the control of an inducible promoter. In some aspects, the first nucleic acid molecule encoding the innate immune inhibitor, e.g., influenza NS1 protein, the second nucleic acid molecule encoding the target mRNA, or both are expressed under the control of tissue specific promoter. In some aspects, the first nucleic acid molecule encoding the innate immune inhibitor, e.g., influenza NS1 protein, the second nucleic acid molecule encoding the target mRNA, or both are expressed under the control of a constitutively active promoter.

Some aspects of the present disclosure are directed to a polynucleotide or a set of polynucleotides comprising a self-replicating target mRNA, wherein the self-replicating target mRNA comprises one or more modified nucleic acid molecule. In some aspects, less than about 50%, less than about 45%, less than about 40%, less than about 35%, less than about 30%, or less than about 25% of the nucleic acids in the polynucleotide or the set of polynucleotides are modified nucleic acid molecules. In some aspects, about 20%, about 21%, about 22%, about 23%, about 24%, about 25%, about 26%, about 27%, about 28%, about 29%, or about 30% of the nucleic acids in the polynucleotide or the set of polynucleotides are modified nucleic acid molecules. In some aspects, the one or more modified nucleic acid molecule is a modified rNTP.

In some aspects, the one or more modified nucleic acid molecules comprise N1-methylpsuedo uracil. In some aspects, the one or more modified nucleic acid molecules comprise 5-methyl cytosine. In some aspects, the one or more modified nucleic acid molecules comprise N1-methylpsuedo uracil and 5-methyl cytosine. Non-limiting examples of additional modified nucleic acid molecules that can be used with the present disclosure include: 6-aza-cytidine, 2-thio-cytidine, α-thio-cytidine, pseudo-iso-cytidine, 5-aminoallyl-uridine, 5-iodo-uridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, O6-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, N4-acetyl-cytidine, 5-methyl-uridine, 5-iodo-cytidine, 1,6-Dimethyl-pseudo-uracil, 1-(optionally substituted C1-C6 Alkyl)-6-(1-propynyl)-pseudo-uracil, 1-(optionally substituted C1-C6 Alkyl)-6-(2-propynyl)-pseudo-uracil, 1-(optionally substituted C1-C6 Alkyl)-6-allyl-pseudo-uracil, 1-(optionally substituted C1-C6 Alkyl)-6-ethynyl-pseudo-uracil, 1-(optionally substituted C1-C6 Alkyl)-6-homoallyl-pseudo-uracil, 1-(optionally substituted C1-C6 Alkyl)-6-vinyl-pseudo-uracil, 1-Methyl-6-(2,2,2-Trifluoroethyl)pseudo-uracil, 1-Methyl-6-(4-morpholino)-pseudo-uracil, 1-Methyl-6-(4-thiomorpholino)-pseudo-uracil, 1-Methyl-6-(optionally substituted phenyl)pseudo-uracil, 1-Methyl-6-amino-pseudo-uracil, 1-Methyl-6-azido-pseudo-uracil, 1-Methyl-6-bromo-pseudo-uracil, 1-Methyl-6-butyl-pseudo-uracil, 1-Methyl-6-chloro-pseudo-uracil, 1-Methyl-6-cyano-pseudo-uracil, 1-Methyl-6-dimethylamino-pseudo-uracil, 1-Methyl-6-ethoxy-pseudo-uracil, 1-Methyl-6-ethylcarboxylate-pseudo-uracil, 1-Methyl-6-ethyl-pseudo-uracil, 1-Methyl-6-fluoro-pseudo-uracil, 1-Methyl-6-formyl-pseudo-uracil, 1-Methyl-6-hydroxyamino-pseudo-uracil, 1-Methyl-6-hydroxy-pseudo-uracil, 1-Methyl-6-iodo-pseudo-uracil, 1-Methyl-6-iso-propyl-pseudo-uracil, 1-Methyl-6-methoxy-pseudo-uracil, 1-Methyl-6-methylamino-pseudo-uracil, 1-Methyl-6-phenyl-pseudo-uracil, 1-Methyl-6-propyl-pseudo-uracil, 1-Methyl-6-tert-butyl-pseudo-uracil, 1-Methyl-6-trifluoromethoxy-pseudo-uracil, 1-Methyl-6-trifluoromethyl-pseudo-uracil, 6-(2,2,2-Trifluoroethyl)-pseudo-uracil, 6-(4-Morpholino)-pseudo-uracil, 6-(4-Thiomorpholino)-pseudo-uracil, 6-(optionally substituted-Phenyl)-pseudo-uracil, 6-Amino-pseudo-uracil, 6-Azido-pseudo-uracil, 6-Bromo-pseudo-uracil, 6-Butyl-pseudo-uracil, 6-Chloro-pseudo-uracil, 6-Cyano-pseudo-uracil, 6-Dimethylamino-pseudo-uracil, 6-Ethoxy-pseudo-uracil, 6-Ethylcarboxylate-pseudo-uracil, 6-Ethyl-pseudo-uracil, 6-Fluoro-pseudo-uracil, 6-Formyl-pseudo-uracil, 6-Hydroxyamino-pseudo-uracil, 6-Hydroxy-pseudo-uracil, 6-Iodo-pseudo-uracil, 6-iso-Propyl-pseudo-uracil, 6-Methoxy-pseudo-uracil, 6-Methylamino-pseudo-uracil, 6-Methyl-pseudo-uracil, 6-Phenyl-pseudo-uracil, 6-Propyl-pseudo-uracil, 6-tert-Butyl-pseudo-uracil, 6-Trifluoromethoxy-pseudo-uracil, 6-Trifluoromethyl-pseudo-uracil, 1-(3-Amino-3-carboxypropyl)pseudo-uracil, 1-(2,2,2-Trifluoroethyl)-pseudo-uracil, 1-(2,4,6-Trimethyl-benzyl)pseudo-uracil, 1-(2,4,6-Trimethyl-phenyl)pseudo-uracil, 1-(2-Amino-2-carboxyethyl)pseudo-uracil, 1-(2-Amino-ethyl)pseudo-uracil, 1-(3-Amino-propyl)pseudo-uracil, 1-(4-Amino-4-carboxybutyl)pseudo-uracil, 1-(4-Amino-benzyl)pseudo-uracil, 1-(4-Amino-butyl)pseudo-uracil, 1-(4-Amino-phenyl)pseudo-uracil, 1-(4-Methoxy-benzyl)pseudo-uracil, 1-(4-Methoxy-phenyl)pseudo-uracil, 1-(4-Methyl-benzyl)pseudo-uracil, 1-(4-Nitro-benzyl)pseudo-uracil, 1(4-Nitro-phenyl)pseudo-uracil, 1-(5-Amino-pentyl)pseudo-uracil, 1-(6-Amino-hexyl)pseudo-uracil, 1-Aminomethyl-pseudo-uracil, 1-Benzyl-pseudo-uracil, 1-Butyl-pseudo-uracil, 1-Cyclobutylmethyl-pseudo-uracil, 1-Cyclobutyl-pseudo-uracil, 1-Cycloheptylmethyl-pseudo-uracil, 1-Cycloheptyl-pseudo-uracil, 1-Cyclohexylmethyl-pseudo-uracil, 1-Cyclohexyl-pseudo-uracil, 1-Cyclooctylmethyl-pseudo-uracil, 1-Cyclooctyl-pseudo-uracil, 1-Cyclopentylmethyl-pseudo-uracil, 1-Cyclopentyl-pseudo-uracil, 1-Cyclopropylmethyl-pseudo-uracil, 1-Cyclopropyl-pseudo-uracil, 1-Ethyl-pseudo-uracil, 1-Hexyl-pseudo-uracil, 1-iso-Propyl-pseudo-uracil 1-Pentyl-pseudo-uracil, 1-Phenyl-pseudo-uracil, 1-Propyl-pseudo-uracil, 1-p-toluyl-pseudo-uracil, 1-tert-Butyl-pseudo-uracil, 1-Trifluoromethyl-pseudo-uracil, 3-(optionally substituted C1-C6 Alkyl)-pseudo-uracil, Pseudo-uracil-N1-2-ethanoic acid, Pseudo-uracil-N1-3-propionic acid, Pseudo-uracil-N1-4-butanoic acid, Pseudo-uracil-N1-5-pentanoic acid, Pseudo-uracil-N1-6-hexanoic acid, Pseudo-uracil-N1-7-heptanoic acid, Pseudo-uracil-N1-methyl-p-benzoic acid, 6-phenyl-pseudo-uracil, 6-azido-pseudo-uracil, Pseudo-uracil-N1-p-benzoic acid, N3-Methyl-pseudo-uracil, 5-Methyl-amino-methyl-uracil, 5-Carboxy-methyl-amino-methyl-uracil, 5-(carboxyhydroxymethyl)uracil methyl ester 5-(carboxyhydroxymethyl)uracil, 2-anhydro-cytosine, 2-anhydro-uracil, 5-Methoxycarbonylmethyl-2-thio-uracil, 5-Methylaminomethyl-2-seleno-uracil, 5-(iso-Pentenylaminomethyl)-uracil, 5-(iso-Pentenylaminomethyl)-2-thio-uracil, 5-(iso-Pentenylaminomethyl)-uracil, 5-Trideuteromethyl-6-deutero-uracil, 5-(2-Chloro-phenyl)-2-thio-cytosine, 5-(4-Amino-phenyl)-2-thio-cytosine, 5-(2-Furanyl)-uracil, 8-Trifluoromethyl-adenosine, 2-Trifluoromethyl-adenosine, 3-Deaza-3-fluoro-adenosine, 3-Deaza-3-bromo-adenosine, 3-Deaza-3-iodo-adenosine, 1-Hydroxymethyl-pseudo-uracil, 1-(2-Hydroxyethyl)-pseudo-uracil, 1-Methoxymethyl-pseudo-uracil, 1-(2-Methoxyethyl)-pseudo-uracil, 1-(2,2-Diethoxyethyl)-pseudo-uracil, 1-(2-Hydroxypropyl)-pseudo-uracil, (2R)-1-(2-Hydroxypropyl)-pseudo-uracil, (2S)-1-(2-Hydroxypropyl)-pseudo-uracil, 1-Cyanomethyl-pseudo-uracil, 1-Morpholinomethyl-pseudo-uracil, 1-Thiomorpholinomethyl-pseudo-uracil, 1-Benzyloxymethyl-pseudo-uracil, 1-(2,2,3,3,3-Pentafluoropropyl)-pseudo-uracil, 1-Thiomethoxymethyl-pseudo-uracil, 1-Methanesulfonylmethyl-pseudo-uracil, 1-Vinyl-pseudo-uracil, 1-Allyl-pseudo-uracil, 1-Homoallyl-pseudo-uracil, 1-Propargyl-pseudo-uracil, 1-(4-Fluorobenzyl)-pseudo-uracil, 1-(4-Chlorobenzyl)-pseudo-uracil, 1-(4-Bromobenzyl)-pseudo-uracil, 1-(4-lodobenzyl)-pseudo-uracil, 1-(4-Methylbenzyl)-pseudo-uracil, 1-(4-Trifluoromethylbenzyl)-pseudo-uracil, 1-(4-Methoxybenzyl)-pseudo-uracil, 1-(4-Trifluoromethoxybenzyl)-pseudo-uracil, 1-(4-Thiomethoxybenzyl)-pseudo-uracil, 1-(4-Methanesulfonylbenzyl)-pseudo-uracil, Pseudo-uracil 1-(4-methylbenzoic acid), Pseudo-uracil 1-(4-methylbenzenesulfonic acid), 1-(2,4,6-Trimethylbenzyl)-pseudouracil, 1-(4-Nitrobenzyl)-pseudo-uracil, 1-(4-Azidobenzyl)-pseudo-uracil, 1-(3,4-Dimethoxybenzyl)-pseudo-uracil, 1-(3,4-Bis-trifluoromethoxybenzyl)-pseudo-uracil, 1-Acetyl-pseudo-uracil, 1-Trifluoroacetyl-pseudo-uracil, 1-Benzoyl-pseudo-uracil, 1-Pivaloyl-pseudo-uracil, 1-(3-Cyclopropyl-prop-2-ynyl)-pseudouracil, Pseudo-uracil 1-methylphosphonic acid diethyl ester, Pseudo-uracil 1-methylphosphonic acid, Pseudo-uracil 1-[3-(2-ethoxy)]propionic acid, Pseudo-uracil 1-[3-{2-(2-ethoxy)-ethoxy}]propionic acid, Pseudo-uracil 1-[3-{2-(2-[2-ethoxy]-ethoxy)-ethoxy}]propionic acid, Pseudo-uracil 1-[3-{2-(2-[2-(2-ethoxy)-ethoxy]-ethoxy)-ethoxy}]propionic acid, Pseudo-uracil 1-[3-{2-(2-[2-{2(2-ethoxy)-ethoxy}-ethoxy]-ethoxy)-ethoxy}]propionic acid, 1-{3-[2-(2-Aminoethoxy)-ethoxy]-propionyl}pseudo-uracil,1-[3-(2-{2-[2-(2-Aminoethoxy)-ethoxy]-ethoxy}-ethoxy)-propionyl]-pseudo-uracil, 1-Biotinyl-pseudo-uracil, 1-Biotinyl-PEG2-pseudo-uracil, 5-(C3-8 cycloalkyl)-cytosine, 5-methyl-N6-acetyl-1-cytosine, 5-(carboxymethyl)-N6-trifluoroacetyl-cytosine trifluoromethyl ester, N6-propionyl-cytosine, 5-monofluoromethyl-cytosine, 5-trifluoromethoxy-cytosine, N6-(1,1,1-trifluoro-propionyl)-cytosine, 4-acetyl-pseudo-isocytosine, 1-ethyl-pseudo-isocytosine, 1-hydroxy-pseudo-isocytosine, or 1-(2,2,2-trifluoroethyl)-pseudo-uracil, 1,6-Dimethyl-pseudo-uracil, 1-(optionally substituted C1-C6 Alkyl)-6-(1-propynyl)-pseudo-uracil, 1-(optionally substituted C1-C6 Alkyl)-6-(2-propynyl)-pseudouracil, 1-(optionally substituted C1-C6 Alkyl)-6-allyl-pseudo-uracil, 1-(optionally substituted C1-C6 Alkyl)-6-ethynyl-pseudo-uracil, 1-(optionally substituted C1-C6 Alkyl)-6-homoallyl-pseudo-uracil, 1-(optionally substituted C1-C6 Alkyl)-6-vinyl-pseudo-uracil, 1-Methyl-6-(2,2,2-Trifluoroethyl)pseudo-uracil, 1-Methyl-6-(4-morpholino)-pseudo-uracil, 1-Methyl-6-(4-thiomorpholino)-pseudo-uracil, 1-Methyl-6-(optionally substituted phenyl)pseudo-uracil, 1-Methyl-6-amino-pseudo-uracil, 1-Methyl-6-azido-pseudo-uracil, 1-Methyl-6-bromo-pseudo-uracil, 1-Methyl-6-butyl-pseudo-uracil, 1-Methyl-6-chloro-pseudo-uracil, 1-Methyl-6-cyano-pseudo-uracil, 1-Methyl-6-dimethylamino-pseudo-uracil, 1-Methyl-6-ethoxy-pseudo-uracil, 1-Methyl-6-ethylcarboxylate-pseudo-uracil, 1-Methyl-6-ethyl-pseudo-uracil, 1-Methyl-6-fluoro-pseudo-uracil, 1-Methyl-6-formyl-pseudo-uracil, 1-Methyl-6-hydroxyamino-pseudo-uracil, 1-Methyl-6-hydroxy-pseudo-uracil, 1-Methyl-6-iodo-pseudo-uracil, 1-Methyl-6-iso-propyl-pseudo-uracil, 1-Methyl-6-methoxy-pseudo-uracil, 1-Methyl-6-methylamino-pseudo-uracil, 1-Methyl-6-phenyl-pseudo-uracil, 1-Methyl-6-propyl-pseudo-uracil, 1-Methyl-6-tert-butyl-pseudo-uracil, 1-Methyl-6-trifluoromethoxy-pseudo-uracil, 1-Methyl-6-trifluoromethyl-pseudo-uracil, 6-(2,2,2-Trifluoroethyl)-pseudo-uracil, 6-(4-Morpholino)-pseudo-uracil, 6-(4-Thiomorpholino)-pseudo-uracil, 6-(Substituted-Phenyl)-pseudo-uracil, 6-Amino-pseudo-uracil, 6-Azido-pseudo-uracil, 6-Bromo-pseudo-uracil, 6-Butyl-pseudo-uracil, 6-Chloro-pseudo-uracil, 6-Cyanopseudo-uracil, 6-Dimethylamino-pseudo-uracil, 6-Ethoxy-pseudo-uracil, 6-Ethylcarboxylate-pseudo-uracil, 6-Ethyl-pseudo-uracil, 6-Fluoro-pseudo-uracil, 6-Formyl-pseudo-uracil, 6-Hydroxyamino-pseudo-uracil, 6-Hydroxy-pseudo-uracil, 6-lodo-pseudo-uracil, 6-iso-Propyl-pseudo-uracil, 6-Methoxy-pseudo-uracil, 6-Methylamino-pseudo-uracil, 6-Methyl-pseudo-uracil, 6-Phenyl-pseudo-uracil, 6-Phenyl-pseudo-uracil, 6-Propyl-pseudo-uracil, 6-tert-Butyl-pseudo-uracil, 6-Trifluoromethoxy-pseudo-uracil, 6-Trifluoromethyl-pseudo-uracil, 1-(3-Amino-3-carboxypropyl)pseudo-uracil, 1-(2,2,2-Trifluoroethyl)-pseudo-uracil, 1-(2,4,6-Trimethyl-benzyl)pseudo-uracil, 1-(2,4,6-Trimethyl-phenyl)pseudo-uracil, 1-(2-Amino-2-carboxyethyl)pseudo-uracil, 1-(2-Amino-ethyl)pseudo-uracil, 1-(3-Amino-propyl)pseudo-uracil, 1-(4-Amino-4-carboxybutyl)pseudo-uracil, 1-(4-Amino-benzyl)pseudo-uracil, 1-(4-Amino-butyl)pseudo-uracil, 1-(4-Amino-phenyl)pseudo-uracil, 1-(4-Methoxy-benzyl)pseudo-uracil, 1-(4-Methoxy-phenyl)pseudo-uracil, 1-(4-Methyl-benzyl)pseudo-uracil, 1-(4-Nitro-benzyl)pseudo-uracil, 1(4-Nitro-phenyl)pseudo-uracil, 1-(5-Amino-pentyl)pseudo-uracil, 1-(6-Amino-hexyl)pseudo-uracil, 1-Aminomethyl-pseudo-uracil, 1-Benzyl-pseudo-uracil, 1-Butyl-pseudo-uracil, 1-Cyclobutylmethyl-pseudo-uracil, 1-Cyclobutyl-pseudo-uracil, 1-Cycloheptylmethyl-pseudo-uracil, 1-Cycloheptyl-pseudo-uracil, 1-Cyclohexylmethyl-pseudo-uracil, 1-Cyclohexyl-pseudo-uracil, 1-Cyclooctylmethyl-pseudo-uracil, 1-Cyclooctyl-pseudo-uracil, 1-Cyclopentylmethyl-pseudo-uracil, 1-Cyclopentyl-pseudo-uracil, 1-Cyclopropylmethyl-pseudo-uracil, 1-Cyclopropyl-pseudo-uracil, 1-Ethyl-pseudo-uracil, 1-Hexyl-pseudo-uracil, 1-iso-Propyl-pseudo-uracil, 1-Pentyl-pseudo-uracil, 1-Phenyl-pseudo-uracil, 1-Propyl-pseudo-uracil, 1-p-tolyl-pseudo-uracil, 1-tert-Butyl-pseudo-uracil, 1-Trifluoromethyl-pseudo-uracil, 3-(optionally substituted C1-C6 Alkyl)-pseudo-uracil, Pseudo-uracil-N1-2-ethanoic acid, Pseudo-uracil-N1-3-propionic acid, Pseudo-uracil-N1-4-butanoic acid, Pseudo-uracil-N1-5-pentanoic acid, Pseudo-uracil-N1-6-hexanoic acid, Pseudo-uracil-N1-7-heptanoic acid, Pseudo-uracil-N1-methyl-p-benzoic acid, 6-phenyl-pseudo-uracil, 6-azido-pseudo-uracil, or Pseudo-uracil-N1-p-benzoic acid, N3-Methyl-pseudo-uracil, 5-Methyl-amino-methyl-uracil, 5-Carboxy-methyl-amino-methyl-uracil, 5-(carboxyhydroxymethyl)uracil methyl ester or 5-(carboxyhydroxymethyl)uracil, and combinations thereof.

In some aspects, the compositions described herein have increased heterologous target mRNA expression in a host cell. In some aspects, the expression of the target mRNA is increased relative to the expression of the target mRNA in the absence of the first nucleic acid molecule encoding the influenza NS1 protein. In some aspects, the expression of the target mRNA is increased relative to the expression of the target mRNA expressed from a replicating RNA not comprising one or more modified nucleic acid molecule. In some aspects, the expression of the target mRNA is increased by at least about 10%, at least about 20%, at least about 25%, at least about 30%, at least about 40%, at least about 50%, at least about 60%, at least about 70%, at least about 75%, at least about 80%, at least about 90%, at least about 100%, at least about 125%, at least about 150%, at least about 175%, at least about 200%, at least about 225%, at least about 250%, at least about 275%, at least about 300%, at least about 350%, at least about 400%, at least about 450%, or at least about 500%, e.g., relative to the expression of the target mRNA in the absence of the first nucleic acid molecule encoding the influenza NS1 protein. In some aspects, the expression of the target mRNA is increased by at least about 100%, e.g., relative to the expression of the target mRNA in the absence of the first nucleic acid molecule encoding the influenza NS1 protein. In some aspects, the expression of the target mRNA is increased by at least about 200%, e.g., relative to the expression of the target mRNA in the absence of the first nucleic acid molecule encoding the influenza NS1 protein. In some aspects, the expression of the target mRNA is increased by at least about 300%, e.g., relative to the expression of the target mRNA in the absence of the first nucleic acid molecule encoding the influenza NS1 protein. In some aspects, the expression of the target mRNA is increased by at least about 400%, e.g., relative to the expression of the target mRNA in the absence of the first nucleic acid molecule encoding the influenza NS1 protein. In some aspects, the expression of the target mRNA is increased by at least about 500%, e.g., relative to the expression of the target mRNA in the absence of the first nucleic acid molecule encoding the influenza NS1 protein.

In some aspects, the expression of the target mRNA is increased by at least about 2-fold, at least about 2.5-fold, at least about 3-fold, at least about 3.5-fold, at least about 4-fold, at least about 4.5-fold, at least about 5-fold, at least about 5.5-fold, or at least about 6-fold, e.g., relative to the expression of the target mRNA in the absence of the first nucleic acid molecule encoding the influenza NS1 protein. In some aspects, the expression of the target mRNA is increased by at least about 2-fold, e.g., relative to the expression of the target mRNA in the absence of the first nucleic acid molecule encoding the influenza NS1 protein. In some aspects, the expression of the target mRNA is increased by at least about 3-fold, e.g., relative to the expression of the target mRNA in the absence of the first nucleic acid molecule encoding the influenza NS1 protein. In some aspects, the expression of the target mRNA is increased by at least about 4-fold, e.g., relative to the expression of the target mRNA in the absence of the first nucleic acid molecule encoding the influenza NS1 protein. In some aspects, the expression of the target mRNA is increased by at least about 5-fold, e.g., relative to the expression of the target mRNA in the absence of the first nucleic acid molecule encoding the influenza NS1 protein. In some aspects, the expression of the target mRNA is increased by at least about 6-fold, e.g., relative to the expression of the target mRNA in the absence of the first nucleic acid molecule encoding the influenza NS1 protein.

In some aspects, the compositions described herein have increased heterologous target mRNA expression and/or increased heterologous target mRNA persistence in a host cell. In some aspects, expression of the target mRNA, e.g., increased expression relative to the expression of the target mRNA in the absence of the first nucleic acid molecule encoding the influenza NS1 protein or relative to the expression of the target mRNA expressed from a replicating RNA not comprising one or more modified nucleic acid molecule, persists for at least about 6 hours, at least about 12 hours, at least about 18 hours, at least about 24 hours, at least about 30 hours, at least about 36 hours, at least about 42 hours, at least about 48 hours, at least about 60 hours, at least about 72 hours, at least about 84 hours, at least about 96 hours, at least about 108 hours, or at least about 120 hours, following transfection of the cell. In some aspects, expression of the target mRNA persists for at least about 48 hours. In some aspects, expression of the target mRNA persists for at least about 48 hours, following transfection of the cell. In some aspects, expression of the target mRNA persists for at least about 60 hours, following transfection of the cell. In some aspects, expression of the target mRNA persists for at least about 72 hours, following transfection of the cell. In some aspects, expression of the target mRNA persists for at least about 84 hours, following transfection of the cell. In some aspects, expression of the target mRNA persists for at least about 96 hours, following transfection of the cell. In some aspects, expression of the target mRNA persists for at least about 108 hours, following transfection of the cell. In some aspects, expression of the target mRNA persists for at least about 120 hours, following transfection of the cell.

II.A. Heterologous Target mRNA

The heterologous target mRNA of the present disclosure can encode any polypeptide or functional RNA of interest. In some aspects, the target mRNA encodes a biologically active polypeptide. In some aspects, the biologically active polypeptide comprises a cytokine, a chemokine, a growth factor, a clotting factor, a hormone, a receptor, a mitogen, an immunoglobulin (e.g., an antibody), an enzyme, or any combination thereof.

In some aspects, the cytokine comprises a cytokine selected from IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP, a fragment thereof, a variant thereof, or any combination thereof. In some aspects, the IL-12 is a single chain IL-12 (scIL-12), protease sensitive IL-12, destabilized IL-12, membrane bound IL-12, intercalated IL-12. In some aspects, the target mRNA encodes an IL-12 polypeptide or a fragment or variant thereof. In some aspects, the IL-12 is a human IL-12. In some aspects, the target mRNA encodes a p35 subunit of IL-12 and a p40 subunit of IL-12. In some aspects, the p35 subunit and the p40 subunit are expressed from a single promoter. In some aspects, the p35 subunit and the p40 subunit are expressed as a single contiguous polypeptide. In some aspects, the p35 subunit and the p40 subunit are linked by one or more covalent bonds. In some aspects, the p35 subunit and the p40 subunit are linked by one or more peptide bonds. In some aspects, the target mRNA encodes a human IL-12 polypeptide comprising a p35 subunit of IL-12 covalently linked to a p40 subunit of IL-12. In some aspects, the p35 subunit of IL-12 and the p40 subunit of IL-12 are linked by a linker comprising one or more amino acid.

In some aspects, the target mRNA comprises a first portion and a second portion, wherein the first portion of the target mRNA encodes a p35 subunit of IL-12 and the second portion of the target mRNA encodes a p40 subunit of IL-12, wherein the first portion and the second portion are separated by an IRES.

In some aspects, the target mRNA encodes a chemokine. Exemplary chemokines include, but are not limited to, CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, and CX3CL1.

In some aspects, the target mRNA encodes an interferon (IFN). Exemplary interferons include, but are not limited to, interferon type I (e.g., IFN-α, IFN-β, IFN-ε, IFN-κ, and IFN-ω), interferon type II (e.g., IFN-γ), and interferon type III. In some aspects, IFN-α is further classified into about 13 subtypes including IFNA1, IFNA2, IFNA4, IFNA5, IFNA6, IFNA7, IFNA8, IFNA10, IFNA13, IFNA14, IFNA16, IFNA17, and IFNA21.

In some aspects, the target mRNA encodes a growth factor. Exemplary growth factors include, but are not limited to, a bone morphogenetic protein (BMP), angiopoietin, CNTF, LIF, M-CSF, G-CSF, GM-CSF, epidermal growth factor (EGF), an ephrin, erythropoietin (EPO), a fibroblast growth factor (FGF), a glial cell line-derived neurotrophic factor (GDNF), growth differentiation factor-9 (GDF9), hepatocyte growth factor (HGF), hepatoma-derived growth factor (HDGF), insulin, insulin-like growth factors Insulin-like growth factor-1 (IGF-1), insulin-like growth factor-2 (IGF-2), keratinocyte growth factor (KGF), migration-stimulating factor (MSF), macrophage-stimulating protein (MSP; also known as hepatocyte growth factor-like protein, HGFLP), myostatin (GDF-8), a neuregulins (NRG), a neurotrophins (e.g., brain-derived neurotrophic factor (BDNF), nerve growth factor (NGF), neurotrophin-3 (NT-3), and neurotrophin-4 (NT-4)), placental growth factor (PGF), platelet-derived growth factor (PDGF), renalase (RNLS), T-cell growth factor (TCGF), thrombopoietin (TPO), transforming growth factors Transforming growth factor alpha (TGF-α), transforming growth factor beta (TGF-β), tumor necrosis factor-alpha (TNF-α), vascular endothelial growth factor (VEGF), and members of the Wnt signaling pathway.

In some aspects, the target mRNA encodes an enzyme. In some aspects, the target mRNA encodes cas9. In some aspects, the target mRNA encodes a zinc finger endonuclease.

In some aspects, the target mRNA encodes a functional RNA. In some aspects, the target mRNA encodes a miRNA, siRNA, shRNA, a dsRNA, antisense oligonucleotide, a guide RNA, or any combination thereof. In some aspects, the target mRNA encodes a guide RNA, e.g., for use in combination with cas9.

### II.B. Influenza NS1

Any influenza NS1 can be used in the compositions and methods of the present disclosure. In some aspects, the influenza NS1 is a type A influenza virus NS1, a type B influenza virus NS1, a type C influenza virus NS1, or a variant thereof. In some aspects, the influenza NS1 is a type A influenza virus NS1. In some aspects, the influenza NS1 is a type B influenza virus NS1. In some aspects, the influenza NS1 is a type C influenza virus NS1.

In some aspects, the influenza NS1 is an H1N1 NS1, H1N2 NS1, H2N2 NS1, H3N2 NS1, H5N1 NS1, H7N9 NS1, H7N7 NS1, H9N2 NS1, H7N2 NS1, H7N3 NS1, H5N2 NS1, H10N7 NS1, a variant thereof, or a combination thereof. In some aspects, the influenza NS1 is an H1N2 NS1. In some aspects, the influenza NS1 is an H2N2 NS1. In some aspects, the influenza NS1 is an H3N2 NS1. In some aspects, the influenza NS1 is an H7N9 NS1. In some aspects, the influenza NS1 is an H7N7 NS1. In some aspects, the influenza NS1 is an H9N2 NS1. In some aspects, the influenza NS1 is an H7N2 NS1. In some aspects, the influenza NS1 is an H7N3 NS1. In some aspects, the influenza NS1 is an H5N2 NS1. In some aspects, the influenza NS1 is an H10N7 NS1.

In some aspects, the influenza NS1 is an H1N1 NS1. In some aspects, the influenza is the H1N1 TX91 variant NS1. In some aspects, the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 1 (Table 1). In some aspects, the influenza NS1 encoded by the first nucleic acid molecule comprises the amino acid sequence encoded by the nucleotide sequence set forth in SEQ ID NO: 1. In some aspects, the influenza NS1 is the H1N1 TX91 variant NS1 encoded by the nucleotide sequence set forth in SEQ ID NO: 1. In some aspects, the first nucleic acid molecule encoding the influenza NS1 comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1, wherein the nucleotide sequence encodes an influenza NS1 protein. In some aspects, the first nucleic acid molecule encoding the influenza NS1 protein comprises the nucleotide sequence set forth in SEQ ID NO: 1, wherein the nucleotide sequence encodes an influenza NS1 protein.

In some aspects, the influenza NS1 is an H5N1 NS1. In some aspects, the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 2 (Table 1). In some aspects, the influenza NS1 encoded by the first nucleic acid molecule comprises the amino acid sequence encoded by the nucleotide sequence set forth in SEQ ID NO: 2. In some aspects, the influenza NS1 is the H1N1 TX91 variant NS1 encoded by the nucleotide sequence set forth in SEQ ID NO: 2. In some aspects, the first nucleic acid molecule encoding the influenza NS1 comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 2, wherein the nucleotide sequence encodes an influenza NS1 protein. In some aspects, the first nucleic acid molecule encoding the influenza NS1 protein comprises the nucleotide sequence set forth in SEQ ID NO: 2, wherein the nucleotide sequence encodes an influenza NS1 protein.

TABLE 1
Influenza NS1 Sequences

Influenza H1N1 TX91 variant NS1 (SEQ ID NO: 1)

| | |
|---|---|
| 1 | AGATTACAAA GGCAACGAAA TAATGACGGC AGCTGCCTCT CAAGGGCTGA CCCGTAAAGG |
| 61 | TGTGTATGCC GTTCGGTACA AGGTGAATGA AAATCCTCTG TACGCACCCA CCTCAGAACA |
| 121 | TGTGAACGTC CTACTGACCC GCACGGAGGA CCGCATCGTG TGGAAAACAC TAGCCGGCGA |
| 181 | CCCATGGATA AAAACACTGA CTGCCAAGTA CCCTGGGAAT TTCACTGCCA CGATAGAGGA |
| 241 | GTGGCAAGCA GAGCATGATG CCATCATGAG GCACATCTTG GAGAGACCGG ACCCTACCGA |
| 301 | CGTCTTCCAG AATAAGGCAA ACGTGTGTTG GGCCAAGGCT TTAGTGCCGG TGCTGAAGAC |
| 361 | CGCTGGCATA GACATGACCA CTGAACAATG GAACACTGTG GATTATTTTG AAACGGACAA |
| 421 | AGCTCACTCA GCAGAGATAG TATTGAACCA ACTATGCGTG AGGTTCTTTG GACTCGATCT |
| 481 | GGACTCCGGT CTATTTTCTG CACCCACTGT TCCGTTATCC ATTAGGAATA ATCACTGGGA |
| 541 | TAACTCCCCG TCGCCTAACA TGTACGGGCT GAATAAAGAA GTGGTCCGTC AGCTCTCTCG |
| 601 | CAGGTACCCA CAACTGCCTC GGGCAGTTGC CACTGGAAGA GTCTATGACA TGAACACTGG |
| 661 | TACACTGCGC AATTATGATC CGCGCATAAA CCTAGTACCT GTAAACAGAA GACTGCCTCA |
| 721 | TGCTTTAGTC CTCCACCATA ATGAACACCC ACAGAGTGAC TTTTCTTCAT TCGTCAGCAA |
| 781 | ATTGAAGGGC AGAACTGTCC TGGTGGTCGG GGAAAAGTTG TCCGTCCCAG GCAAAATGGT |
| 841 | TGACTGGTTG TCAGACCGGC CTGAGGCTAC CTTCAGAGCT CGGCTGGATT TAGGCATCCC |
| 901 | AGGTGATGTG CCCAAATATG ACATAATATT TGTTAATGTG AGGACCCCAT ATAAATACCA |
| 961 | TCACTATCAG CAGTGTGAAG ACCATGCCAT TAAGCTTAGC ATGTTGACCA AGAAAGCTTG |
| 1021 | TCTGCATCTG AATCCCGGCG GAACCTGTGT CAGCATAGGT TATGGTTACG CTGACAGGGC |
| 1081 | CAGCGAAAGC ATCATTGGTG CTATAGCGCG GCAGTTCAAG TTTTCCCGGG TATGCAAACC |
| 1141 | GAAATCCTCA CTTGAAGAGA CGGAAGTTCT GTTTGTATTC ATTGGGTACG ATCGCAAGGC |
| 1201 | CCGTACGCAC AATCCTTACA AGCTTTCATC AACCTTGACC AACATTTATA CAGGTTCCAG |
| 1261 | ACTCCACGAA GCCGGATGTG CACCCTCATA TCATGTGGTG CGAGGGGATA TTGCCACGGC |
| 1321 | CACCGAAGGA GTGATTATAA ATGCTGCTAA CAGCAAAGGA CAACCTGGCG GAGGGGTGTG |
| 1381 | CGGAGCGCTG TATAAGAAAT TCCCGGAAAG CTTCGATTTA CAGCCGATCG AAGTAGGAAA |
| 1441 | AGCGCGACTG GTCAAAGGTG CAGCTAAACA TATCATTCAT GCCGTAGGAC CAAACTTCAA |
| 1501 | CAAAGTTTCG GAGGTTGAAG GTGACAAACA GTTGGCAGAG GCTTATGAGT CCATCGCTAA |
| 1561 | GATTGTCAAC GATAACAATT ACAAGTCAGT AGCGATTCCA CTGTTGTCCA CCGGCATCTT |
| 1621 | TTCCGGGAAC AAAGATCGAC TAACCCAATC ATTGAACCAT TTGCTGACAG CTTTAGACAC |
| 1681 | CACTGATGCA GATGTAGCCA TATACTGCAG GGACAAGAAA TGGGAAATGA CTCTCAAGGA |
| 1741 | AGCAGTGGCT AGGAGAGAAG CAGTGGAGGA GATATGCATA TCCGACGACT CTTCAGTGAC |
| 1801 | AGAACCTGAT GCAGAGCTGG TGAGGGTGCA TCCGAAGAGT TCTTTGGCTG GAAGGAAGGG |
| 1861 | CTACAGCACA AGCGATGGCA AAACTTTCTC ATATTTGGAA GGGACCAAGT TTCACCAGGC |
| 1921 | GGCCAAGGAT ATAGCAGAAA TTAATGCCAT GTGGCCCGTT GCAACGGAGG CCAATGAGCA |
| 1981 | GGTATGCATG TATATCCTCG GAGAAAGCAT GAGCAGTATT AGGTCGAAAT GCCCCGTCGA |
| 2041 | AGAGTCGGAA GCCTCCACAC CACCTAGCAC GCTGCCTTGC TTGTGCATCC ATGCCATGAC |
| 2101 | TCCAGAAAGA GTACAGCGCC TAAAAGCCTC ACGTCCAGAA CAAATTACTG TGTGCTCATC |
| 2161 | CTTTCCATTG CCGAAGTATA GAATCACTGG TGTGCAGAAG ATCCAATGCT CCCAGCCTAT |
| 2221 | ATTGTTCTCA CCGAAAGTGC CTGCGTATAT TCATCCAAGG AAGTATCTCG TGGAAACACC |
| 2281 | ACCGGTAGAC GAGACTCCGG AGCCATCGGC AGAGAACCAA TCCACAGAGG GGACACCTGA |
| 2341 | ACAACCACCA CTTATAACCG AGGATGAGAC CAGGACTAGA ACGCCTGAGC CGATCATCAT |
| 2401 | CGAAGAGGAA GAAGAGGATA GCATAAGTTT GCTGTCAGAT GGCCCGACCC ACCAGGTGCT |
| 2461 | GCAAGTCGAG GCAGACATTC ACGGGCCGCC CTCTGTATCT AGCTCATCCT GGTCCATTCC |
| 2521 | TCATGCATCC GACTTTGATG TGGACAGTTT ATCCATACTT GACACCCTGG AGGGAGCTAG |
| 2581 | CGTGACCAGC GGGGCAACGT CAGCCGAGAC TAACTCTTAC TTCGCAAAGA GTATGGAGTT |
| 2641 | TCTGGCGCGA CCGGTGCCTG CGCCTCGAAC AGTATTCAGG AACCCTCCAC ATCCCGCTCC |
| 2701 | GCGCACAAGA ACACCGTCAC TTGCACCCAG CAGGGCCTGC TCGAGAACCA GCCTAGTTTC |
| 2761 | CACCCCGCCA GGCGTGAATA GGGTGATCAC TAGAGAGGAG CTCGAGGCGC TTACCCCGTC |
| 2821 | ACGCACTCCT AGCAGGTCGG TCTCGAGAAC CAGCCTGGTC TCCAACCCGC CAGGCGTAAA |
| 2881 | TAGGGTGATT ACAAGAGAGG AGTTTGAGGC GTTCGTAGCA CAACAACAAT GACGGTTTGA |
| 2941 | TGCGGGTGCA TACATCTTTT CCTCCGACAC CGGTCAAGGG CATTTACAAC AAAAATCAGT |
| 3001 | AAGGCAAACG GTGCTATCCG AAGTGGTGTT GGAGAGGACC GAATTGGAGA TTTCGTATGC |
| 3061 | CCCGCGCCTC GACCAAGAAA AAGAAGAATT ACTACGCAAG AAATTACAGT TAAATCCCAC |
| 3121 | ACCTGCTAAC AGAAGCAGAT ACCAGTCCAG GAAGGTGGAG AACATGAAAG CCATAACAGC |
| 3181 | TAGACGTATT CTGCAAGGCC TAGGGCATTA TTTGAAGGCA GAAGGAAAAG TGGAGTGCTA |
| 3241 | CCGAACCCTG CATCCTGTTC CTTTGTATTC ATCTAGTGTG AACCGTGCCT TTTCAAGCCC |
| 3301 | CAAGGTCGCA GTGGAAGCCT GTAACGCCAT GTTGAAAGAG AACTTTCCGA CTGTGGCTTC |
| 3361 | TTACTGTATT ATTCCAGAGT ACGATGCCTA TTTGGACATG GTTGACGGAG CTTCATGCTG |
| 3421 | CTTAGACACT GCCAGTTTTT GCCCTGCAAA GCTGCGCAGC TTTCCAAAGA AACACTCCTA |
| 3481 | TTTGGAACCC ACAATACGAT CGGCAGTGCC TTCAGCGATC CAGAACACGC TCCAGAACGT |
| 3541 | CCTGGCAGCT GCCACAAAAA GAAATTGCAA TGTCACGCAA ATGAGAGAAT TGCCCGTATT |
| 3601 | GGATTCGGCG GCCTTTAATG TGGAATGCTT CAAGAAATAT GCGTGTAATA ATGAATATTG |
| 3661 | GGAAACGTTT AAAGAAAACC CCATCAGGCT TACTGAAGAA AACGTGGTAA ATTACATTAC |
| 3721 | CAAATTAAAA GGACCAAAAG CTGCTGCTCT TTTTGCGAAG ACACATAATT TGAATATGTT |
| 3781 | GCAGGACATA CCAATGGACA GGTTTGTAAT GGACTTAAAG AGAGACGTGA AAGTGACTCC |
| 3841 | AGGAACAAAA CATACTGAAG AACGGCCCAA GGTACAGGTG ATCCAGGCTG CCGATCCGCT |
| 3901 | AGCAACAGCG TATCTGTGCG GAATCCACCG AGAGCTGGTT AGGAGATTAA ATGCGGTCCT |
| 3961 | GCTTCCGAAC ATTCATACAC TGTTTGATAT GTCGGCTGAA GACTTTGACG CTATTATAGC |
| 4021 | CGAGCACTTC CAGCCTGGGG ATTGTGTTCT GGAAACTGAC ATCGCGTCGT TTGATAAAAG |
| 4081 | TGAGGACGAC GCCATGGCTC TGACCGCGTT AATGATTCTG GAAGACTTAG GTGTGGACGC |
| 4141 | AGAGCTGTTG ACGCTGATTG AGGCGGCTTT CGGCGAAATT TCATCAATAC ATTTGCCCAC |
| 4201 | TAAAACTAAA TTTAAATTCG GAGCCATGAT GAAATCTGGA ATGTTCCTCA CACTGTTTGT |
| 4261 | GAACACAGTC ATTAACATTG TAATCGCAAG CAGAGTGTTG AGAGAACGGC TAACCGGATC |
| 4321 | ACCATGTGCA GCATTCATTG GAGATGACAA TATCGTGAAA GGAGTCAAAT CGGACAAATT |
| 4381 | AATGGCAGAC AGGTGCGCCA CCTGGTTGAA TATGGAAGTC AAGATTATAG ATGCTGTGGT |
| 4441 | GGGCGAGAAA GCGCCTTATT TCTGTGGAGG GTTTATTTTG TGTGACTCCG TGACCGGCAC |
| 4501 | AGCGTGCCGT GTGGCAGACC CCCTAAAAAG GCTGTTTAAG CTTGGCAAAC CTCTGGCAGC |
| 4561 | AGACGATGAA CATGATGATG ACAGGAGAAG GGCATTGCAT GAAGAGTCAA CACGCTGGAA |
| 4621 | CCGAGTGGGT ATTCTTTCAG AGCTGTGCAA GGCAGTAGAA TCAAGGTATG AAACCGTAGG |
| 4681 | AACTTCCATC ATAGTTATGG CCATGACTAC TCTAGCTAGC AGTGTTAAAT CATTCAGCTA |
| 4741 | CCTGAGAGGG GCCCCTATAA CTCTCTACGG CTAACCTGAA TGGACTACGA CATAGTCTAG |
| 4801 | TCCGCCAAGG CCACCatgga cagcaacacg gtgtcaagct tccaggtcga ctgcttcctg |
| 4861 | tggcacgtgc gcaagcaggt ggcggaccag gagctgggcg acgccccgtt cctcgaccgg |
| 4921 | ctgcgccgcg accagaagtc cctgaagggg cggggcagca cgctcggcct gaacatcgag |
| 4981 | accgcgacgt gcgtcggcaa gcagatcgtg gagcggatcc tcaaggagga gagcgacgag |
| 5041 | gccttccgca tgaccatggc gagcgccctg gcgtcgcgct acctcacgga catgacgatc |
| 5101 | gaggagatga gccgggactg gttcatgctc atgcccaagc agaaggtggc ggggcccctc |
| 5161 | tgcgtgcgga tggaccaggc catcatggac aagaacatca tcctgaaggc gaacttcagc |
| 5221 | gtgatcttcg accgcctgga gacgctgacc ctcctgcgcg cattcaccga ggagggggcg |
| 5281 | atcgtcggcg agatcagccc gctgccgtcc ctgccggggc acacgaacga ggacgtcaag |
| 5341 | aacgccatcg gcgtcctcat cgggggcctc gagtggaacg acaacaccgt ccgggtctcc |
| 5401 | gagacgctcc agcggttcgc gtggcggagc agcaacgaga acggcgggcc gccgctcacc |
| 5461 | cccacgcaga agcggaagat ggccggaaag atccgctccg aggtctgaTA ATATGTTACG |
| 5521 | TGCAAAGGTG ATTGTCACCC CCCGAAAGAC CATATTGTGA CACACCCTCA GTATCACGCC |
| 5581 | CAAACATTTA CAGCCGCGGT GTCAAAAACC GCGTGGACGT GGTTAACATC CCTGCTGGGA |
| 5641 | GGATCAGCCG TAATTATTAT AATTGGCTTG GTGCTGGCTA CTATTGTGGC CATGTACGTG |
| 5701 | CTGACCAACC AGAAACATAA TTGAATACAG CAGCAATTGG CAAGCTGCTT ACATAGAACT |
| 5761 | CGCGGCGATT GGCATGCCGC CTTAAAATTT TTATTTTATT TTTCTTTTCT TTTCCGAATC |
| 5821 | GGATTTTGTT TTTAATATTT CAAAAAAAAA AAAAAAAAAA AAAAAAAAAA AAAAAAAAAA |
| 5881 | ATAGGGATAA CAGGGTAATT GAGCAAAAGG CCAGCAAAAG GCCAGGAACC GTAAAAAGGC |
| 5941 | CGCGTTGCTG GCGTTTTTCC ATAGGCTCCG CCCCCCTGAC GAGCATCACA AAAATCGACG |
| 6001 | CTCAAGTCAG AGGTGGCGAA ACCCGACAGG ACTATAAAGA TACCAGGCGT TTCCCCCTGG |
| 6061 | AAGCTCCCTC GTGCGCTCTC CTGTTCCGAC CCTGCCGCTT ACCGGATACC TGTCCGCCTT |
| 6121 | TCTCCCTTCG GGAAGCGTGG CGCTTTCTCA TAGCTCACGC TGTAGGTATC TCAGTTCGGT |
| 6181 | GTAGGTCGTT CGCTCCAAGC TGGGCTGTGT GCACGAACCC CCCGTTCAGC CCGACCGCTG |
| 6241 | CGCCTTATCC GGTAACTATC GTCTTGAGTC CAACCCGGTA AGACACGACT TATCGCCACT |
| 6301 | GGCAGCAGCC ACTGGTAACA GGATTAGCAG AGCGAGGTAT GTAGGCGGTG CTACAGAGTT |
| 6361 | CTTGAAGTGG TGGCCTAACT ACGGCTACAC TAGAAGAACA GTATTTGGTA TCTGCGCTCT |
| 6421 | GCTGAAGCCA GTTACCTTCG GAAAAAGAGT TGGTAGCTCT TGATCCGGCA AACAAACCAC |
| 6481 | CGCTGGTAGC GGTGGTTTTT TTGTTTGCAA GCAGCAGATT ACGCGCAGAA AAAAAGGATC |
| 6541 | TCAAGAAGAT CCTTTGATCT TTTCTACGGG GTCTGACGCT CAGTGGAACG AAAACTCACG |
| 6601 | TTAAGGGATT TTGGTCATGA GATTATCAAA AAGGATCTTC ACCTAGATCC TTTTAAATTA |
| 6661 | AAAATGAAGT TTTAAATCAA TCTAAAGTAT ATATGAGTAA ACTTGGTCTG ACAGTTACCA |
| 6721 | ATGCTTAATC AGTGAGGCAC CTATCTCAGC GATCTGTCTA TTTCGTTCAT CCATAGTTGC |
| 6781 | CTGACTCCCC GTCGTGTAGA TAACTACGAT ACGGGAGGGC TTACCATCTG GCCCCAGTGC |
| 6841 | TGCAATGATA CCGCGAGACC CACGCTCACC GGCTCCAGAT TTATCAGCAA TAAACCAGCC |
| 6901 | AGCCGGAAGG GCCGAGCGCA GAAGTGGTCC TGCAACTTTA TCCGCCTCCA TCCAGTCTAT |
| 6961 | TAATTGTTGC CGGGAAGCTA GAGTAAGTAG TTCGCCAGTT AATAGTTTGC GCAACGTTGT |
| 7021 | TGCCATTGCT ACAGGCATCG TGGTGTCACG CTCGTCGTTT GGTATGGCTT CATTCAGCTC |
| 7081 | CGGTTCCCAA CGATCAAGGC GAGTTACATG ATCCCCCATG TTGTGCAAAA AAGCGGTTAG |
| 7141 | CTCCTTCGGT CCTCCGATCG TTGTCAGAAG TAAGTTGGCC GCAGTGTTAT CACTCATGGT |
| 7201 | TATGGCAGCA CTGCATAATT CTCTTACTGT CATGCCATCC GTAAGATGCT TTTCTGTGAC |
| 7261 | TGGTGAGTAC TCAACCAAGT CATTCTGAGA ATAGTGTATG CGGCGACCGA GTTGCTCTTG |
| 7321 | CCCGGCGTCA ATACGGGATA ATACCGCGCC ACATAGCAGA ACTTTAAAAG TGCTCATCAT |
| 7381 | TGGAAAACGT TCTTCGGGGC GAAAACTCTC AAGGATCTTA CCGCTGTTGA GATCCAGTTC |
| 7441 | GATGTAACCC ACTCGTGCAC CCAACTGATC TTCAGCATCT TTTACTTTCA CCAGCGTTTC |
| 7501 | TGGGTGAGCA AAAACAGGAA GGCAAAATGC CGCAAAAAAG GGAATAAGGG CGACACGGAA |
| 7561 | ATGTTGAATA CTCATACTCT TCCTTTTTCA ATATTATTGA AGCATTTATC AGGGTTATTG |
| 7621 | TCTCATGAGC GGATACATAT TTGAATGTAT TTAGAAAAAT AAACAAATAG GGGTTCCGCG |
| 7681 | CACATTTCCC CGAAAAGTGC CACCTGACGT TAGGGATAAC AGGGTAATTA ATACGACTCA |
| 7741 | CTATAATGGG CGGCGCATGA GAGAAGCCCA GACCAATTAC CTACCCAAAA TGGAGAAAGT |
| 7801 | TCACGTTGAC ATCGAGGAAG ACAGCCCATT CCTCAGAGCT TTGCAGCGGA GCTTCCCGCA |
| 7861 | GTTTGAGGTA GAAGCCAAGC AGGTCACTGA TAATGACCAT GCTAATGCCA GAGCGTTTTC |
| 7921 | GCATCTGGCT TCAAAACTGA TCGAAACGGA GGTGGACCCA TCCGACACGA TCCTTGACAT |
| 7981 | TGGAAGTGCG CCCGCCCGCA GAATGTATTC TAAGCACAAG TATCATTGTA TCTGTCCGAT |
| 8041 | GAGATGTGCG GAAGATCCGG ACAGATTGTA TAAGTATGCA ACTAAGCTGA AGAAAAACTG |
| 8101 | TAAGGAAATA ACTGATAAGG AATTGGACAA GAAAATGAAG GAGCTCGCCG CCGTCATGAG |
| 8161 | CGACCCTGAC CTGGAAACTG AGACTATGTG CCTCCACGAC GACGAGTCGT GTCGCTACGA |
| 8221 | AGGGCAAGTC GCTGTTTACC AGGATGTATA CGCGGTTGAC GGACCGACAA GTCTCTATCA |
| 8281 | CCAAGCCAAT AAGGGAGTTA GAGTCGCCTA CTGGATAGGC TTTGACACCA CCCCTTTTAT |
| 8341 | GTTTAAGAAC TTGGCTGGAG CATATCCATC ATACTCTACC AACTGGGCCG ACGAAACCGT |
| 8401 | GTTAACGGCT CGTAACATAG GCCTATGCAG CTCTGACGTT ATGGAGCGGT CACGTAGAGG |
| 8461 | GATGTCCATT CTTAGAAAGA AGTATTTGAA ACCATCCAAC AATGTTCTAT TCTCTGTTGG |
| 8521 | CTCGACCATC TACCACGAGA AGAGGGACTT ACTGAGGAGC TGGCACCTGC CGTCTGTATT |
| 8581 | TCACTTACGT GGCAAGCAAA ATTACACATG TCGGTGTGAG ACTATAGTTA GTTGCGACGG |
| 8641 | GTACGTCGTT AAAAGAATAG CTATCAGTCC AGGCCTGTAT GGGAAGCCTT CAGGCTATGC |
| 8701 | TGCTACGATG CACCGCGAGG GATTCTTGTG CTGCAAAGTG ACAGACACAT TGAACGGGGA |
| 8761 | GAGGGTCTCT TTTCCCGTGT GCACGTATGT GCCAGCTACA TTGTGTGACC AAATGACTGG |
| 8821 | CATACTGGCA ACAGATGTCA GTGCGGACGA CGCGCAAAAA CTGCTGGTTG GGCTCAACCA |
| 8881 | GCGTATAGTC GTCAACGGTC GCACCCAGAG AAACACCAAT ACCATGAAAA ATTACCTTTT |
| 8941 | GCCCGTAGTG GCCCAGGCAT TTGCTAGGTG GGCAAAGGAA TATAAGGAAG ATCAAGAAGA |
| 9001 | TGAAAGGCCA CTAGGACTAC GAGATAGACA GTTAGTCATG GGGTGTTGTT GGGCTTTTAG |
| 9061 | AAGGCACAAG ATAACATCTA TTTATAAGCG CCCGGATACC CAAACCATCA TCAAAGTGAA |
| 9121 | CAGCGATTTC CACTCATTCG TGCTGCCCAG GATAGGCAGT AACACATTGG AGATCGGGCT |
| 9181 | GAGAACAAGA ATCAGGAAAA TGTTAGAGGA GCACAAGGAG CCGTCACCTC TCATTACCGC |
| 9241 | CGAGGACGTA CAAGAAGCTA AGTGCGCAGC CGATGAGGCT AAGGAGGTGC GTGAAGCCGA |
| 9301 | GGAGTTGCGC GCAGCTCTAC CACCTTTGGC AGCTGATGTT GAGGAGCCCA CTCTGGAAGC |
| 9361 | CGATGTCGAC TTGATGTTAC AAGAGGCTGG GGCCGGCTCA GTGGAGACAC CTCGTGGCTT |
| 9421 | GATAAAGGTT ACCAGCTACG ATGGCGAGGA CAAGATCGGC TCTTACGCTG TGCTTTCTCC |
| 9481 | GCAGGCTGTA CTCAAGAGTG AAAAATTATC TTGCATCCAC CCTCTCGCTG AACAAGTCAT |
| 9541 | AGTGATAACA CACTCTGGCC GAAAAGGGCG TTATGCCGTG GAACCATACC ATGGTAAAGT |
| 9601 | AGTGGTGCCA GAGGGACATG CAATACCCGT CCAGGACTTT CAAGCTCTGA GTGAAAGTGC |
| 9661 | CACCATTGTG TACAACGAAC GTGAGTTCGT AAACAGGTAC CTGCACCATA TTGCCACACA |
| 9721 | TGGAGGAGCG CTGAACACTG ATGAAGAATA TTACAAAACT GTCAAGCCCA GCGAGCACGA |
| 9781 | CGGCGAATAC CTGTACGACA TCGACAGGAA ACAGTGCGTC AAGAAAGAAC TAGTCACTGG |
| 9841 | GCTAGGGCTC ACAGGCGAGC TGGTGGATCC TCCCTTCCAT GAATTCGCCT ACGAGAGTCT |
| 9901 | GAGAACACGA CCAGCCGCTC CTTACCAAGT ACCAACCATA GGGGTGTATG GCGTGCCAGG |
| 9961 | ATCAGGCAAG TCTGGCATCA TTAAAAGCGC AGTCACCAAA AAAGATCTAG TGGTGAGCGC |
| 10021 | CAAGAAAGAA AACTGTGCAG AAATTATAAG GGACGTCAAG AAAATGAAAG GGCTGGACGT |
| 10081 | CAATGCCAGA ACTGTGGACT CAGTGCTCTT GAATGGATGC AAACACCCCG TAGAGACCCT |
| 10141 | GTATATTGAC GAAGCTTTTG CTTGTCATGC AGGTACTCTC AGAGCGCTCA TAGCCATTAT |
| 10201 | AAGACCTAAA AAGGCAGTGC TCTGCGGGGA TCCCAAACAG TGCGGTTTTT TTAACATGAT |
| 10261 | GTGCCTGAAA GTGCATTTTA ACCACGAGAT TTGCACACAA GTCTTCCACA AAAGCATCTC |
| 10321 | TCGCCGTTGC ACTAAATCTG TGACTTCGGT CGTCTCAACC TTGTTTTACG ACAAAAAAAT |
| 10381 | GAGAACGACG AATCCGAAAG AGACTAAGAT TGTGATTGAC ACTACCGGCA GTACCAAACC |
| 10441 | TAAGCAGGAC GATCTCATTC TCACTTGTTT CAGAGGGTGG GTGAAGCAGT TGCAAAT |

Influenza H5N1 NS1 (SEQ ID NO: 2)

| | |
|---|---|
| 1 | TAGATTACAA AGGCAACGAA ATAATGACGG CAGCTGCCTC TCAAGGGCTG ACCCGTAAAG |
| 61 | GTGTGTATGC CGTTCGGTAC AAGGTGAATG AAAATCCTCT GTACGCACCC ACCTCAGAAC |
| 121 | ATGTGAACGT CCTACTGACC CGCACGGAGG ACCGCATCGT GTGGAAAACA CTAGCCGGCG |
| 181 | ACCCATGGAT AAAAACACTG ACTGCCAAGT ACCCTGGGAA TTTCACTGCC ACGATAGAGG |
| 241 | AGTGGCAAGC AGAGCATGAT GCCATCATGA GGCACATCTT GGAGAGACCG GACCCTACCG |
| 301 | ACGTCTTCCA GAATAAGGCA AACGTGTGTT GGGCCAAGGC TTTAGTGCCG GTGCTGAAGA |
| 361 | CCGCTGGCAT AGACATGACC ACTGAACAAT GGAACACTGT GGATTATTTT GAAACGGACA |
| 421 | AAGCTCACTC AGCAGAGATA GTATTGAACC AACTATGCGT GAGGTTCTTT GGACTCGATC |
| 481 | TGGACTCCGG TCTATTTTCT GCACCCACTG TTCCGTTATC CATTAGGAAT AATCACTGGG |
| 541 | ATAACTCCCC GTCGCCTAAC ATGTACGGGC TGAATAAAGA AGTGGTCCGT CAGCTCTCTC |
| 601 | GCAGGTACCC ACAACTGCCT CGGGCAGTTG CCACTGGAAG AGTCTATGAC ATGAACACTG |
| 661 | GTACACTGCG CAATTATGAT CCGCGCATAA ACCTAGTACC TGTAAACAGA AGACTGCCTC |
| 721 | ATGCTTTAGT CCTCCACCAT AATGAACACC CACAGAGTGA CTTTTCTTCA TTCGTCAGCA |
| 781 | AATTGAAGGG CAGAACTGTC CTGGTGGTCG GGGAAAAGTT GTCCGTCCCA GGCAAAATGG |
| 841 | TTGACTGGTT GTCAGACCGG CCTGAGGCTA CCTTCAGAGC TCGGCTGGAT TTAGGCATCC |
| 901 | CAGGTGATGT GCCCAAATAT GACATAATAT TTGTTAATGT GAGGACCCCA TATAAATACC |
| 961 | ATCACTATCA GCAGTGTGAA GACCATGCCA TTAAGCTTAG CATGTTGACC AAGAAAGCTT |
| 1021 | GTCTGCATCT GAATCCCGGC GGAACCTGTG TCAGCATAGG TTATGGTTAC GCTGACAGGG |
| 1081 | CCAGCGAAAG CATCATTGGT GCTATAGCGC GGCAGTTCAA GTTTTCCCGG GTATGCAAAC |
| 1141 | CGAAATCCTC ACTTGAAGAG ACGGAAGTTC TGTTTGTATT CATTGGGTAC GATCGCAAGG |
| 1201 | CCCGTACGCA CAATCCTTAC AAGCTTTCAT CAACCTTGAC CAACATTTAT ACAGGTTCCA |
| 1261 | GACTCCACGA AGCCGGATGT GCACCCTCAT ATCATGTGGT GCGAGGGGAT ATTGCCACGG |
| 1321 | CCACCGAAGG AGTGATTATA AATGCTGCTA ACAGCAAAGG ACAACCTGGC GGAGGGGTGT |
| 1381 | GCGGAGCGCT GTATAAGAAA TTCCCGGAAA GCTTCGATTT ACAGCCGATC GAAGTAGGAA |
| 1441 | AAGCGCGACT GGTCAAAGGT GCAGCTAAAC ATATCATTCA TGCCGTAGGA CCAAACTTCA |
| 1501 | ACAAAGTTTC GGAGGTTGAA GGTGACAAAC AGTTGGCAGA GGCTTATGAG TCCATCGCTA |
| 1561 | AGATTGTCAA CGATAACAAT TACAAGTCAG TAGCGATTCC ACTGTTGTCC ACCGGCATCT |
| 1621 | TTTCCGGGAA CAAAGATCGA CTAACCCAAT CATTGAACCA TTTGCTGACA GCTTTAGACA |
| 1681 | CCACTGATGC AGATGTAGCC ATATACTGCA GGGACAAGAA ATGGGAAATG ACTCTCAAGG |
| 1741 | AAGCAGTGGC TAGGAGAGAA GCAGTGGAGG AGATATGCAT ATCCGACGAC TCTTCAGTGA |
| 1801 | CAGAACCTGA TGCAGAGCTG GTGAGGGTGC ATCCGAAGAG TTCTTTGGCT GGAAGGAAGG |
| 1861 | GCTACAGCAC AAGCGATGGC AAAACTTTCT CATATTTGGA AGGGACCAAG TTTCACCAGG |
| 1921 | CGGCCAAGGA TATAGCAGAA ATTAATGCCA TGTGGCCCGT TGCAACGGAG GCCAATGAGC |
| 1981 | AGGTATGCAT GTATATCCTC GGAGAAAGCA TGAGCAGTAT TAGGTCGAAA TGCCCCGTCG |
| 2041 | AAGAGTCGGA AGCCTCCACA CCACCTAGCA CGCTGCCTTG CTTGTGCATC CATGCCATGA |
| 2101 | CTCCAGAAAG AGTACAGCGC CTAAAAGCCT CACGTCCAGA ACAAATTACT GTGTGCTCAT |
| 2161 | CCTTTCCATT GCCGAAGTAT AGAATCACTG GTGTGCAGAA GATCCAATGC TCCCAGCCTA |
| 2221 | TATTGTTCTC ACCGAAAGTG CCTGCGTATA TTCATCCAAG GAAGTATCTC GTGGAAACAC |
| 2281 | CACCGGTAGA CGAGACTCCG GAGCCATCGG CAGAGAACCA ATCCACAGAG GGGACACCTG |
| 2341 | AACAACCACC ACTTATAACC GAGGATGAGA CCAGGACTAG AACGCCTGAG CCGATCATCA |
| 2401 | TCGAAGAGGA AGAAGAGGAT AGCATAAGTT TGCTGTCAGA TGGCCCGACC CACCAGGTGC |
| 2461 | TGCAAGTCGA GGCAGACATT CACGGGCCGC CCTCTGTATC TAGCTCATCC TGGTCCATTC |
| 2521 | CTCATGCATC CGACTTTGAT GTGGACAGTT TATCCATACT TGACACCCTG GAGGGAGCTA |
| 2581 | GCGTGACCAG CGGGGCAACG TCAGCCGAGA CTAACTCTTA CTTCGCAAAG AGTATGGAGT |
| 2641 | TTCTGGCGCG ACCGGTGCCT GCGCCTCGAA CAGTATTCAG GAACCCTCCA CATCCCGCTC |
| 2701 | CGCGCACAAG AACACCGTCA CTTGCACCCA GCAGGGCCTG CTCGAGAACC AGCCTAGTTT |
| 2761 | CCACCCCGCC AGGCGTGAAT AGGGTGATCA CTAGAGAGGA GCTCGAGGCG CTTACCCCGT |
| 2821 | CACGCACTCC TAGCAGGTCG GTCTCGAGAA CCAGCCTGGT CTCCAACCCG CCAGGCGTAA |
| 2881 | ATAGGGTGAT TACAAGAGAG GAGTTTGAGG CGTTCGTAGC ACAACAACAA TGACGGTTTG |
| 2941 | ATGCGGGTGC ATACATCTTT TCCTCCGACA CCGGTCAAGG GCATTTACAA CAAAAATCAG |
| 3001 | TAAGGCAAAC GGTGCTATCC GAAGTGGTGT TGGAGAGGAC CGAATTGGAG ATTTCGTATG |
| 3061 | CCCCGCGCCT CGACCAAGAA AAAGAAGAAT TACTACGCAA GAAATTACAG TTAAATCCCA |
| 3121 | CACCTGCTAA CAGAAGCAGA TACCAGTCCA GGAAGGTGGA GAACATGAAA GCCATAACAG |
| 3181 | CTAGACGTAT TCTGCAAGGC CTAGGGCATT ATTTGAAGGC AGAAGGAAAA GTGGAGTGCT |
| 3241 | ACCGAACCCT GCATCCTGTT CCTTTGTATT CATCTAGTGT GAACCGTGCC TTTTCAAGCC |
| 3301 | CCAAGGTCGC AGTGGAAGCC TGTAACGCCA TGTTGAAAGA GAACTTTCCG ACTGTGGCTT |
| 3361 | CTTACTGTAT TATTCCAGAG TACGATGCCT ATTTGGACAT GGTTGACGGA GCTTCATGCT |
| 3421 | GCTTAGACAC TGCCAGTTTT TGCCCTGCAA AGCTGCGCAG CTTTCCAAAG AAACACTCCT |
| 3481 | ATTTGGAACC CACAATACGA TCGGCAGTGC CTTCAGCGAT CCAGAACACG CTCCAGAACG |
| 3541 | TCCTGGCAGC TGCCACAAAA AGAAATTGCA ATGTCACGCA AATGAGAGAA TTGCCCGTAT |
| 3601 | TGGATTCGGC GGCCTTTAAT GTGGAATGCT TCAAGAAATA TGCGTGTAAT AATGAATATT |
| 3661 | GGGAAACGTT TAAAGAAAAC CCCATCAGGC TTACTGAAGA AAACGTGGTA AATTACATTA |
| 3721 | CCAAATTAAA AGGACCAAAA GCTGCTGCTC TTTTTGCGAA GACACATAAT TTGAATATGT |
| 3781 | TGCAGGACAT ACCAATGGAC AGGTTTGTAA TGGACTTAAA GAGAGACGTG AAAGTGACTC |
| 3841 | CAGGAACAAA ACATACTGAA GAACGGCCCA AGGTACAGGT GATCCAGGCT GCCGATCCGC |
| 3901 | TAGCAACAGC GTATCTGTGC GGAATCCACC GAGAGCTGGT TAGGAGATTA AATGCGGTCC |
| 3961 | TGCTTCCGAA CATTCATACA CTGTTTGATA TGTCGGCTGA AGACTTTGAC GCTATTATAG |
| 4021 | CCGAGCACTT CCAGCCTGGG GATTGTGTTC TGGAAACTGA CATCGCGTCG TTTGATAAAA |
| 4081 | GTGAGGACGA CGCCATGGCT CTGACCGCGT TAATGATTCT GGAAGACTTA GGTGTGGACG |
| 4141 | CAGAGCTGTT GACGCTGATT GAGGCGGCTT TCGGCGAAAT TTCATCAATA CATTTGCCCA |
| 4201 | CTAAAACTAA ATTTAAATTC GGAGCCATGA TGAAATCTGG AATGTTCCTC ACACTGTTTG |
| 4261 | TGAACACAGT CATTAACATT GTAATCGCAA GCAGAGTGTT GAGAGAACGG CTAACCGGAT |
| 4321 | CACCATGTGC AGCATTCATT GGAGATGACA ATATCGTGAA AGGAGTCAAA TCGGACAAAT |
| 4381 | TAATGGCAGA CAGGTGCGCC ACCTGGTTGA ATATGGAAGT CAAGATTATA GATGCTGTGG |
| 4441 | TGGGCGAGAA AGCGCCTTAT TTCTGTGGAG GGTTTATTTT GTGTGACTCC GTGACCGGCA |
| 4501 | CAGCGTGCCG TGTGGCAGAC CCCCTAAAAA GGCTGTTTAA GCTTGGCAAA CCTCTGGCAG |
| 4561 | CAGACGATGA ACATGATGAT GACAGGAGAA GGGCATTGCA TGAAGAGTCA ACACGCTGGA |
| 4621 | ACCGAGTGGG TATTCTTTCA GAGCTGTGCA AGGCAGTAGA ATCAAGGTAT GAAACCGTAG |
| 4681 | GAACTTCCAT CATAGTTATG GCCATGACTA CTCTAGCTAG CAGTGTTAAA TCATTCAGCT |
| 4741 | ACCTGAGAGG GGCCCCTATA ACTCTCTACG GCTAACCTGA ATGGACTACG ACATAGTCTA |
| 4801 | GTCCGCCAAG GCCACCatgg acagcaacac ggtgtcctcc ttccaggtgg actgcttcct |
| 4861 | ctggcacgtg cgcaagcgct tcgccgacca ggagctgggc gacgccccct tcctggaccg |
| 4921 | ccttcgccgg gaccagaagt ccctgcgggg ccggggcagc acgcttggcc tggacatccg |
| 4981 | cacggccacc cgggagggga agcacatcgt ggagcggatc ctggaggagg agtcggacga |
| 5041 | ggccctgaag atgacgatcg cgagcgtgcc cgcgccccgg tacctaaccg agatgacgct |
| 5101 | ggaggagatg agcagggact ggctgatgct catccccaag cagaaggtga ccgggtccct |
| 5161 | ctgcatacgc atggaccagg ccatcatgga caaggacatc atcctgaagg ccaacttcag |
| 5221 | cgtcatcttt aaccggctgg aggccctcat cctgctccgc gccttcaccg acgagggggc |
| 5281 | cattgtgggg gagatcagcc ccctccccag cctgccgggc cacaccgagg aggacgtcaa |
| 5341 | gaacgccatc ggggtcctca tcggcggcct cgagtggaac gacaacaccg tccgcgtgag |
| 5401 | cgagaccctc cagcggttca cgtggcgcag ctctgacgag aacggccgga gccccctccc |
| 5461 | gcccaagcag aagcggaaga tggagcggac gatcgagccc gaggtgtgaT AATATGTTAC |
| 5521 | GTGCAAAGGT GATTGTCACC CCCCGAAAGA CCATATTGTG ACACACCCTC AGTATCACGC |
| 5581 | CCAAACATTT ACAGCCGCGG TGTCAAAAAC CGCGTGGACG TGGTTAACAT CCCTGCTGGG |
| 5641 | AGGATCAGCC GTAATTATTA TAATTGGCTT GGTGCTGGCT ACTATTGTGG CCATGTACGT |
| 5701 | GCTGACCAAC CAGAAACATA ATTGAATACA GCAGCAATTG GCAAGCTGCT TACATAGAAC |
| 5761 | TCGCGGCGAT TGGCATGCCG CCTTAAAATT TTTATTTTAT TTTTCTTTTC TTTTCCGAAT |
| 5821 | CGGATTTTGT TTTTAATATT TCAAAAAAAA AAAAAAAAAA AAAAAAAAAA AAAAAAAAAA |
| 5881 | AATAGGGATA ACAGGGTAAT TGAGCAAAAG GCCAGCAAAA GGCCAGGAAC CGTAAAAAGG |
| 5941 | CCGCGTTGCT GGCGTTTTTC CATAGGCTCC GCCCCCCTGA CGAGCATCAC AAAAATCGAC |
| 6001 | GCTCAAGTCA GAGGTGGCGA AACCCGACAG GACTATAAAG ATACCAGGCG TTTCCCCCTG |
| 6061 | GAAGCTCCCT CGTGCGCTCT CCTGTTCCGA CCCTGCCGCT TACCGGATAC CTGTCCGCCT |
| 6121 | TTCTCCCTTC GGGAAGCGTG GCGCTTTCTC ATAGCTCACG CTGTAGGTAT CTCAGTTCGG |
| 6181 | TGTAGGTCGT TCGCTCCAAG CTGGGCTGTG TGCACGAACC CCCCGTTCAG CCCGACCGCT |
| 6241 | GCGCCTTATC CGGTAACTAT CGTCTTGAGT CCAACCCGGT AAGACACGAC TTATCGCCAC |
| 6301 | TGGCAGCAGC CACTGGTAAC AGGATTAGCA GAGCGAGGTA TGTAGGCGGT GCTACAGAGT |
| 6361 | TCTTGAAGTG GTGGCCTAAC TACGGCTACA CTAGAAGAAC AGTATTTGGT ATCTGCGCTC |
| 6421 | TGCTGAAGCC AGTTACCTTC GGAAAAAGAG TTGGTAGCTC TTGATCCGGC AAACAAACCA |
| 6481 | CCGCTGGTAG CGGTGGTTTT TTTGTTTGCA AGCAGCAGAT TACGCGCAGA AAAAAAGGAT |
| 6541 | CTCAAGAAGA TCCTTTGATC TTTTCTACGG GGTCTGACGC TCAGTGGAAC GAAAACTCAC |
| 6601 | GTTAAGGGAT TTTGGTCATG AGATTATCAA AAAGGATCTT CACCTAGATC CTTTTAAATT |
| 6661 | AAAAATGAAG TTTTAAATCA ATCTAAAGTA TATATGAGTA AACTTGGTCT GACAGTTACC |
| 6721 | AATGCTTAAT CAGTGAGGCA CCTATCTCAG CGATCTGTCT ATTTCGTTCA TCCATAGTTG |
| 6781 | CCTGACTCCC CGTCGTGTAG ATAACTACGA TACGGGAGGG CTTACCATCT GGCCCCAGTG |
| 6841 | CTGCAATGAT ACCGCGAGAC CCACGCTCAC CGGCTCCAGA TTTATCAGCA ATAAACCAGC |
| 6901 | CAGCCGGAAG GGCCGAGCGC AGAAGTGGTC CTGCAACTTT ATCCGCCTCC ATCCAGTCTA |
| 6961 | TTAATTGTTG CCGGGAAGCT AGAGTAAGTA GTTCGCCAGT TAATAGTTTG CGCAACGTTG |
| 7021 | TTGCCATTGC TACAGGCATC GTGGTGTCAC GCTCGTCGTT TGGTATGGCT TCATTCAGCT |
| 7081 | CCGGTTCCCA ACGATCAAGG CGAGTTACAT GATCCCCCAT GTTGTGCAAA AAAGCGGTTA |
| 7141 | GCTCCTTCGG TCCTCCGATC GTTGTCAGAA GTAAGTTGGC CGCAGTGTTA TCACTCATGG |
| 7201 | TTATGGCAGC ACTGCATAAT TCTCTTACTG TCATGCCATC CGTAAGATGC TTTTCTGTGA |
| 7261 | CTGGTGAGTA CTCAACCAAG TCATTCTGAG AATAGTGTAT GCGGCGACCG AGTTGCTCTT |
| 7321 | GCCCGGCGTC AATACGGGAT AATACCGCGC CACATAGCAG AACTTTAAAA GTGCTCATCA |
| 7381 | TTGGAAAACG TTCTTCGGGG CGAAAACTCT CAAGGATCTT ACCGCTGTTG AGATCCAGTT |
| 7441 | CGATGTAACC CACTCGTGCA CCCAACTGAT CTTCAGCATC TTTTACTTTC ACCAGCGTTT |
| 7501 | CTGGGTGAGC AAAAACAGGA AGGCAAAATG CCGCAAAAAA GGGAATAAGG GCGACACGGA |
| 7561 | AATGTTGAAT ACTCATACTC TTCCTTTTTC AATATTATTG AAGCATTTAT CAGGGTTATT |
| 7621 | GTCTCATGAG CGGATACATA TTTGAATGTA TTTAGAAAAA TAAACAAATA GGGGTTCCGC |
| 7681 | GCACATTTCC CCGAAAAGTG CCACCTGACG TTAGGGATAA CAGGGTAATT AATACGACTC |
| 7741 | ACTATAATGG GCGGCGCATG AGAGAAGCCC AGACCAATTA CCTACCCAAA ATGGAGAAAG |
| 7801 | TTCACGTTGA CATCGAGGAA GACAGCCCAT TCCTCAGAGC TTTGCAGCGG AGCTTCCCGC |
| 7861 | AGTTTGAGGT AGAAGCCAAG CAGGTCACTG ATAATGACCA TGCTAATGCC AGAGCGTTTT |
| 7921 | CGCATCTGGC TTCAAAACTG ATCGAAACGG AGGTGGACCC ATCCGACACG ATCCTTGACA |
| 7981 | TTGGAAGTGC GCCCGCCCGC AGAATGTATT CTAAGCACAA GTATCATTGT ATCTGTCCGA |
| 8041 | TGAGATGTGC GGAAGATCCG GACAGATTGT ATAAGTATGC AACTAAGCTG AAGAAAAACT |
| 8101 | GTAAGGAAAT AACTGATAAG GAATTGGACA AGAAAATGAA GGAGCTCGCC GCCGTCATGA |
| 8161 | GCGACCCTGA CCTGGAAACT GAGACTATGT GCCTCCACGA CGACGAGTCG TGTCGCTACG |
| 8221 | AAGGGCAAGT CGCTGTTTAC CAGGATGTAT ACGCGGTTGA CGGACCGACA AGTCTCTATC |
| 8281 | ACCAAGCCAA TAAGGGAGTT AGAGTCGCCT ACTGGATAGG CTTTGACACC ACCCCTTTTA |
| 8341 | TGTTTAAGAA CTTGGCTGGA GCATATCCAT CATACTCTAC CAACTGGGCC GACGAAACCG |
| 8401 | TGTTAACGGC TCGTAACATA GGCCTATGCA GCTCTGACGT TATGGAGCGG TCACGTAGAG |
| 8461 | GGATGTCCAT TCTTAGAAAG AAGTATTTGA AACCATCCAA CAATGTTCTA TTCTCTGTTG |
| 8521 | GCTCGACCAT CTACCACGAG AAGAGGGACT TACTGAGGAG CTGGCACCTG CCGTCTGTAT |
| 8581 | TTCACTTACG TGGCAAGCAA AATTACACAT GTCGGTGTGA GACTATAGTT AGTTGCGACG |
| 8641 | GGTACGTCGT TAAAAGAATA GCTATCAGTC CAGGCCTGTA TGGGAAGCCT TCAGGCTATG |
| 8701 | CTGCTACGAT GCACCGCGAG GGATTCTTGT GCTGCAAAGT GACAGACACA TTGAACGGGG |
| 8761 | AGAGGGTCTC TTTTCCCGTG TGCACGTATG TGCCAGCTAC ATTGTGTGAC CAAATGACTG |
| 8821 | GCATACTGGC AACAGATGTC AGTGCGGACG ACGCGCAAAA ACTGCTGGTT GGGCTCAACC |
| 8881 | AGCGTATAGT CGTCAACGGT CGCACCCAGA GAAACACCAA TACCATGAAA AATTACCTTT |
| 8941 | TGCCCGTAGT GGCCCAGGCA TTTGCTAGGT GGGCAAAGGA ATATAAGGAA GATCAAGAAG |
| 9001 | ATGAAAGGCC ACTAGGACTA CGAGATAGAC AGTTAGTCAT GGGGTGTTGT TGGGCTTTTA |
| 9061 | GAAGGCACAA GATAACATCT ATTTATAAGC GCCCGGATAC CCAAACCATC ATCAAAGTGA |
| 9121 | ACAGCGATTT CCACTCATTC GTGCTGCCCA GGATAGGCAG TAACACATTG GAGATCGGGC |
| 9181 | TGAGAACAAG AATCAGGAAA ATGTTAGAGG AGCACAAGGA GCCGTCACCT CTCATTACCG |
| 9241 | CCGAGGACGT ACAAGAAGCT AAGTGCGCAG CCGATGAGGC TAAGGAGGTG CGTGAAGCCG |
| 9301 | AGGAGTTGCG CGCAGCTCTA CCACCTTTGG CAGCTGATGT TGAGGAGCCC ACTCTGGAAG |
| 9361 | CCGATGTCGA CTTGATGTTA CAAGAGGCTG GGGCCGGCTC AGTGGAGACA CCTCGTGGCT |
| 9421 | TGATAAAGGT TACCAGCTAC GATGGCGAGG ACAAGATCGG CTCTTACGCT GTGCTTTCTC |
| 9481 | CGCAGGCTGT ACTCAAGAGT GAAAAATTAT CTTGCATCCA CCCTCTCGCT GAACAAGTCA |
| 9541 | TAGTGATAAC ACACTCTGGC CGAAAAGGGC GTTATGCCGT GGAACCATAC CATGGTAAAG |
| 9601 | TAGTGGTGCC AGAGGGACAT GCAATACCCG TCCAGGACTT TCAAGCTCTG AGTGAAAGTG |
| 9661 | CCACCATTGT GTACAACGAA CGTGAGTTCG TAAACAGGTA CCTGCACCAT ATTGCCACAC |
| 9721 | ATGGAGGAGC GCTGAACACT GATGAAGAAT ATTACAAAAC TGTCAAGCCC AGCGAGCACG |
| 9781 | ACGGCGAATA CCTGTACGAC ATCGACAGGA AACAGTGCGT CAAGAAAGAA CTAGTCACTG |
| 9841 | GGCTAGGGCT CACAGGCGAG CTGGTGGATC CTCCCTTCCA TGAATTCGCC TACGAGAGTC |
| 9901 | TGAGAACACG ACCAGCCGCT CCTTACCAAG TACCAACCAT AGGGGTGTAT GGCGTGCCAG |
| 9961 | GATCAGGCAA GTCTGGCATC ATTAAAAGCG CAGTCACCAA AAAAGATCTA GTGGTGAGCG |
| 10021 | CCAAGAAAGA AAACTGTGCA GAAATTATAA GGGACGTCAA GAAAATGAAA GGGCTGGACG |
| 10081 | TCAATGCCAG AACTGTGGAC TCAGTGCTCT TGAATGGATG CAAACACCCC GTAGAGACCC |
| 10141 | TGTATATTGA CGAAGCTTTT GCTTGTCATG CAGGTACTCT CAGAGCGCTC ATAGCCATTA |
| 10201 | TAAGACCTAA AAAGGCAGTG CTCTGCGGGG ATCCCAAACA GTGCGGTTTT TTTAACATGA |
| 10261 | TGTGCCTGAA AGTGCATTTT AACCACGAGA TTTGCACACA AGTCTTCCAC AAAAGCATCT |
| 10321 | CTCGCCGTTG CACTAAATCT GTGACTTCGG TCGTCTCAAC CTTGTTTTAC GACAAAAAAA |
| 10381 | TGAGAACGAC GAATCCGAAA GAGACTAAGA TTGTGATTGA CACTACCGGC AGTACCAAAC |
| 10441 | CTAAGCAGGA CGATCTCATT CTCACTTGTT TCAGAGGGTG GGTGAAGCAG TTGCAAA |

### II.C. Vectors

In some aspects, one or more of the first nucleic acid molecule encoding the influenza NS1 protein and the second nucleic acid molecule encoding the target mRNA are present in a vector. Any vectors can be used in the compositions and method disclosed herein. In some aspects, the vector comprises a viral vector, a mammalian vector, bacterial vector, or a combination or variant thereof. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

In some aspects, the vector comprises a replicon. In some aspects, a replicon derived from an alphavirus comprises a positive-strand RNA that encodes RNA-dependent RNA polymerases that simultaneously transcribe therapeutic payloads and self-amplify the replicon on entry into the cytoplasm. In some aspects, the vector comprises a replicon, wherein the replicon comprises (1) UTRs of the parent virus and non-structural proteins and subgenomic promoter (SGP) of the parent virus and (2) the first nucleic acid molecule encoding the influenza NS1 protein, the second nucleic acid molecule encoding the target mRNA, or both the first nucleic acid molecule encoding the influenza NS1 protein and the second nucleic acid molecule encoding the target mRNA. Any replicon can be used in the compositions and methods disclosed herein. In some aspects, the replicon comprises a Venezuelan equine encephalitis (VEE) replicon or a derivative or portion thereof. In some aspects, the replicon comprises one or more point mutation relative to the parent viral replicon, e.g., one or more point mutation relative to the VEE replicon sequence. Modifications to the sequence of the replicon can be used to increase the expression of the target mRNA, to increase the persistence of the target mRNA (e.g., by reducing an immune response against the target mRNA or the polynucleotide encoding the target mRNA and/or by reducing type I interferon activity in a target cell, e.g., a target cancer cell), or both. In some aspects, the vector comprises a VEE replicon, wherein the VEE replicon comprises a Q739L mutation relative to the parent VEE replicon. In some aspects, the first vector, the second vector, or both comprise one or more additional regulatory elements. In some aspects, the first vector, the second vector, or both comprise a tissue specific promoter, a tissue specific enhancer, a tissue specific silencer, or any combination thereof.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) the second nucleic acid molecule encoding the target mRNA; and (v) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) a P2A linker; (v) the second nucleic acid molecule encoding the target mRNA; and (vi) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) the second nucleic acid molecule encoding the target mRNA; (v) an E1 sequence; and (vi) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) a P2A linker; (v) the second nucleic acid molecule encoding the target mRNA; (vi) an E1 sequence; and (vii) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) the second nucleic acid molecule encoding the target mRNA, wherein the target mRNA encodes a human IL-12 polypeptide; and (v) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding the influenza NS1 protein; (iv) a P2A linker; (v) the second nucleic acid molecule encoding the target mRNA, wherein the target mRNA encodes a human IL-12 polypeptide; (vi) an El sequence; and (vii) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H1N1 TX91 variant NS1 or an H5N1 NS1; (iv) the second nucleic acid molecule encoding the target mRNA; and (v) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H1N1 TX91 variant NS1 or an H5N1 NS1; (iv) a P2A linker; (v) the second nucleic acid molecule encoding the target mRNA; (vi) an E1 sequence; and (vii) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H1N1 TX91 variant NS1 or an H5N1 NS1; (iv) the second nucleic acid molecule encoding the target mRNA, wherein the target mRNA encodes a human IL-12 polypeptide; and (v) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H1N1 TX91 variant NS1 or an H5N1 NS1; (iv) a P2A linker; (v) the second nucleic acid molecule encoding the target mRNA, wherein the target mRNA encodes a human IL-12 polypeptide; (vi) an E1 sequence; and (vii) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H1N1 TX91 variant NS1; (iv) the second nucleic acid molecule encoding the target mRNA, wherein the target mRNA encodes a human IL-12 polypeptide; and (v) a 3′UTR from a parent replicon, e.g., a 3′UTR from VEE. In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1; (iv) the second nucleic acid molecule encoding the target mRNA, wherein the target mRNA encodes a human IL-12 polypeptide; and (v) a 3′UTR from a parent replicon, e.g., a 3′UTR from VEE. In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule comprising the nucleotide sequence set forth in SEQ ID NO: 1; (iv) the second nucleic acid molecule encoding the target mRNA, wherein the target mRNA encodes a human IL-12 polypeptide; and (v) a 3′UTR from a parent replicon, e.g., a 3′UTR from VEE.

In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule encoding an H5N1 NS1; (iv) a P2A linker; (v) the second nucleic acid molecule encoding the target mRNA, wherein the target mRNA encodes a human IL-12 polypeptide; (vi) an E1 sequence; and (vii) a 3′UTR from a parent replicon, e.g., a 3′UTR from VEE. In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 2; (iv) the second nucleic acid molecule encoding the target mRNA, wherein the target mRNA encodes a human IL-12 polypeptide; and (v) a 3′UTR from a parent replicon, e.g., a 3′UTR from VEE. In some aspects, the vector comprises (i) a 5′UTR from a parent replicon, e.g., a 5′UTR from VEE; (ii) one or more non-structural protein (nsP) from a parent replicon, e.g., one or more nsP from VEE, e.g., nsP2, nsP3, and nsP4 from VEE; (iii) a first nucleic acid molecule comprising the nucleotide sequence set forth in SEQ ID NO: 2; (iv) the second nucleic acid molecule encoding the target mRNA, wherein the target mRNA encodes a human IL-12 polypeptide; and (v) a 3′UTR from a parent replicon, e.g., a 3′UTR from VEE.

### I.D. Lipid Nanoparticles

Some aspects of the present disclosure are directed to a lipid nanoparticle comprising, e.g., encapsulating, a polynucleotide or a set of polynucleotides disclosed herein. In some aspects, the polynucleotide or the set of polynucleotides disclosed herein is packaged and/or delivered in a lipid nanoparticle. Accordingly, in some aspects, the present disclosure relates to a polynucleotide described herein encapsulated by lipid nanoparticles, the composition thereof, and use of the composition thereof.

A “lipid nanoparticle” (LNP), as used herein, refers to a vesicle, such as a spherical vesicle, having a contiguous lipid bilayer. Lipid nanoparticles can be used in methods by which pharmaceutical therapies are delivered to targeted locations. Non-limiting examples of LNPs include liposomes, bolaamphiphiles, solid lipid nanoparticles (SLN), nanostructured lipid carriers (NLC), and monolayer membrane structures (e.g., archaeosomes and micelles).

In some aspects, the lipid nanoparticle comprises one or more types of lipids. A lipid, as used herein, refers to a group of organic compounds that include, but are not limited to, esters of fatty acids and in some aspects are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. Non-limiting examples of lipids include triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). In some aspects, the one or more types of lipids in the LNP comprises a cationic lipid. In some aspects, the one or more types of lipids in the LNP comprises a lipidoid, e.g., TT3.

Such lipids useful for the present disclosure include, but are not limited to N1,N3,N5-tris(3-(didodecylamino)propyl)benzene-1,3,5-tricarboxamide (TT3), N-(2,3-dioleoyloxy)propyl)-N,N,N-trimethylammonium chloride (DOTAP); lipofectamine; 1,2-DiLinoleyloxy-N,N-dimethylaminopropane (DLinDMA), 1,2-Dilinolenyloxy-N,N-dimethylaminopropane (DLenDMA); dioctadecyldimethylammonium (DODMA), Distearyldimethylammonium (DSDMA), N,N-dioleyl-N,N,-dimethylammonium chloride (DODAC); N-(2,3-dioleyloxy)propyl)-N,N,N-trimethylammonium chloride (DOTMA); N-N-distearyl-N,N-dimethylammonium bromide (DDAB); 3-(N—(N′,N′-dimethylaminoethane)-carbamoyl)cholesterol (DC-Chol) and N-(1,2-dimyristyloxprop-3-yl)-N,N-dimethyl-N-hydroxyethyl ammonium bromide (DMRIE).

In some aspects of the disclosure, the lipids, e.g., lipidoid, is TT3. TT3, as used herein, is capable of forming lipid nanoparticles for delivery of various biologic active agents into the cells.

In some aspects of the disclosure, the cationic lipid is DOTAP. DOTAP, as used herein, is also capable of forming lipid nanoparticles. DOTAP can be used for the highly efficient transfection of DNA including yeast artificial chromosomes (YACs) into eukaryotic cells for transient or stable gene expression, and is also suitable for the efficient transfer of other negatively charged molecules, such as RNA, oligonucleotides, nucleotides, ribonucleoprotein (RNP) complexes, and proteins into research samples of mammalian cells.

In some aspects of the disclosure, the cationic lipid is lipofectamine. Lipofectamine, as used herein, is a common transfection reagent, produced and sold by Invitrogen, used in molecular and cellular biology. It is used to increase the transfection efficiency of RNA (including mRNA and siRNA) or plasmid DNA into in vitro cell cultures by lipofection. Lipofectamine contains lipid subunits that can form liposomes or lipid nanoparticles in an aqueous environment, which entrap the transfection payload, e.g., modRNA. The RNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

In some aspects, LNPs are composed primarily of cationic lipids along with other lipid ingredients. These typically include other lipid molecules belonging but not limited to the phophatidylcholine (PC) class (e.g., 1,s-Distearoyl-sn-glycero-3-phophocholine (DSPC), and 1,2-Dioleoyl-sn-glycero-3-phophoethanolamine (DOPE), sterols (e.g., cholesterol) and Polyethylene glycol (PEG)-lipid conjugates (e.g., 1,2-distearoyl-sn-glycero-3-phosphoethanolamine-N-[folate(polyethylene glycol)-2000 (DSPE-PEG2000), and C14-PEG2000. Table 2 shows the formulation of exemplary LNPs, TT3-LNP and DOTAP-LNP.

TABLE 2

| DOTAP-LNP | DOTAP | DSPC | Cholesterol | DSPE-PEG2000 |
|---|---|---|---|---|
| Molar ratio | 40 | 10 | 48 | 2 |
| TT3-LNP | TT3 | DOPE | Cholesterol | C14-PEG2000 |
| Molar Ratio | 20 | 30 | 40 | 0.75 |

In some aspects, the LNP comprises C14-PEG2000. In some aspects, C14-PEG2000 comprises 1,2-dimyristoyl-rac-glycero-3-methoxypolyethylene glycol-2000 (DMG-PEG2000), 1,2-dimyristoyl-sn-glycero-3-phosphoethanolamine-N-[methoxy(polyethylene glycol)-2000](DMPE-PEG2000), or both. In some aspects, the C14-PEG2000 (or other lipid ingredients disclosed herein) can be embedded in the LNP prior to the encapsulation of the polynucleotide. In some aspects, the C14-PEG2000 (or other lipid ingredients disclosed herein) can be added to the LNP after the encapsulation of the polynucleotide.

Particle size of lipid nanoparticles can affect drug release rate, bio-distribution, mucoadhesion, cellular uptake of water and buffer exchange to the interior of the nanoparticles, and protein diffusion. In some aspects of the disclosure, the diameter of the LNPs ranges from about 30 to about 500 nm. In some aspects of the disclosure, the diameter of the LNPs ranges from about 30 to about 500 nm, about 50 to about 400 nm, about 70 to about 300 nm, about 100 to about 200 nm, about 100 to about 175 nm, or about 100 to about 160 nm. In some aspects of the disclosure, the diameter of the LNPs ranges from 100-160 nm. In some aspects of the disclosure, the diameter of the LNPs can be about 30 nm, about 40 nm, about 50 nm, about 60 nm, about 70 nm, about 80 nm, about 90 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 130 nm, about 140 nm, about 150 nm, or about 160 nm. In some aspects, the lipid nanoparticle has a diameter of about 140 nm.

Zeta potential is a measure of the effective electric charge on the lipid nanoparticle surface. The magnitude of the zeta potential provides information about particle stability. In some aspects of the disclosure, the zeta potential of the LNPs ranges from about 3 to about 6 mv. In some aspects of the disclosure, the zeta potential of the LNPs can be about 3 mv, about 3.1 mv, about 3.2 mv, about 3.3 mv, about 3.4 mv, about 3.5 mv, about 3.6 mv, about 3.7 mv, about 3.8 mv, about 3.9 mv, about 4 mv, about 4.1 mv, about 4.2 mv, about 4.3 mv, about 4.4 mv, about 4.5 my, about 4.6 mv, about 4.7 mv, about 4.8 mv, about 4.9 mv, about 5 my, about 5.1 mv, about 5.2 mv, about 5.3 mv, about 5.4 mv, about 5.5 my, about 5.6 mv, about 5.7 mv, about 5.8 mv, about 5.9 mv, or about 6 mv.

In some aspects, the disclosure is related to encapsulated polynucleotide or set of polynucleotides with lipid nanoparticles (LNPs). In some aspects of the disclosure, the mass ratio between the lipid of LNPs and the polynucleotide or set of polynucleotides ranges from about 1:2 to about 15:1. In some aspects, the mass ratio between the lipid and the polynucleotide or set of polynucleotides can be about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, or about 15:1. In some aspects of the disclosure, the mass ratio between the lipid and the polynucleotide or set of polynucleotides is about 10:1.

### II.E. Cells

In some aspects, provided herein are cells that have been modified to comprise a polynucleotide or set of polynucleotides described herein. In some aspects, the cells comprise a vector or a set of vectors that comprise(s) a polynucleotide or set of polynucleotides described herein. In some aspects, the cells comprise a lipid nanoparticle that comprises a polynucleotide or set of polynucleotides described herein. In some aspects, the cell is a cancer cell. In some aspects, the cell is an immune cell. In some aspects, immune cell comprises a T cell (e.g., CD4+ T cell, CD8+ T cell, or both), a natural killer cell (NK cell), a tumor infiltrating lymphocyte, a dendritic cell, a B cell, a bone marrow cell, a monocyte, or a PBMC.

In some aspects, the cells described herein (i.e., comprising a polynucleotide or a set of polynucleotides of the present disclosure or a vector or a set of vectors comprising the same) can produce the (i) human influenza NS1 and (ii) the target heterologous mRNA. In some aspects, the cells described herein (i.e., comprising a polynucleotide or a set of polynucleotides of the present disclosure or a vector or a set of vectors comprising the same) can produce the (i) human influenza NS1 and (ii) the target heterologous mRNA in vivo. For instance, in some aspects, a polynucleotide or a set of polynucleotides of the present disclosure or a vector or a set of vectors comprising the same can be introduced into a cell ex vivo (e.g., via transfection), and then the cell can be administered to a subject (e.g., adoptive cell therapy), wherein the (i) human influenza NS1 and (ii) the target heterologous mRNA are produced in the subject after the administration. In some aspects, a polynucleotide or a set of polynucleotides of the present disclosure or a vector or a set of vectors comprising the same can be administered to a subject, e.g., as part of a gene therapy. In some aspects, the cells described herein (i.e., comprising a polynucleotide or a set of polynucleotides of the present disclosure or a vector or a set of vectors comprising the same) can produce the (i) human influenza NS1 and (ii) the target heterologous mRNA both in vitro and in vivo.

In some aspects, the cell is a host cell. In some aspects, the host cell is a eukaryotic cell. In some aspects, the host cell is selected from the group consisting of a mammalian cell, an insect cell, a yeast cell, a transgenic mammalian cell, a plant cell, and any combination thereof. In some aspects, the host cell is a prokaryotic cell. In some aspects, the prokaryotic cell is a bacterial cell.

In some aspects, the host cell is a mammalian cell. Non-limiting examples of mammalian host cells that are suitable for the present disclosure include: CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, HBK, NSO, HT1080, HsS78Bst cells, and combinations thereof.

### II.F. Pharmaceutical Compositions

As is apparent from the present disclosure, any of the polynucleotides, vectors, lipid nanoparticles, and cells described herein (also referred to herein as “active compounds”) can be incorporated into pharmaceutical compositions suitable for administration. Accordingly, in some aspects, the pharmaceutical composition comprises the active compound and a pharmaceutically acceptable excipient.

As used herein, the term “pharmaceutically acceptable excipient” (also referred to herein as “pharmaceutically acceptable carrier”) comprises any and all solvents, dispersion media, coatings, antibacterial and antifungal agents, isotonic and absorption delaying agents, and the like, compatible with pharmaceutical administration. The use of such media and agents for pharmaceutically active compounds is well known in the art. Except insofar as any conventional media or agent is incompatible with the active compound, use thereof in the compositions is contemplated. Supplementary active compounds can also be incorporated into the compositions.

In some aspects, disclosed herein is a pharmaceutical composition comprising (a) a polynucleotide or a set of polynucleotides described herein and (b) a pharmaceutically acceptable excipient. In some aspects, disclosed herein is a pharmaceutical composition comprising (a) a vector or a set of vectors as described herein and (b) a pharmaceutically acceptable excipient. In some aspects, disclosed herein is a pharmaceutical composition comprising (a) a lipid nanoparticle as described herein and (b) a pharmaceutically acceptable excipient. In some aspects, disclosed herein is a pharmaceutical composition comprising (a) a cell as described herein (e.g., modified to comprise a polynucleotide or a set of polynucleotides of the present disclosure) and (b) a pharmaceutically acceptable excipient.

A pharmaceutical composition of the present disclosure is formulated to be compatible with its intended route of administration. In some aspects, a suitable route of administration that can be used with the present disclosure comprises intramuscular administration. In some aspects, a suitable route of administration includes intranasal administration. Additional examples of suitable routes of administration include parenteral, e.g., intravenous, intradermal, subcutaneous, oral, transdermal (topical), and transmucosal, and any combination thereof. Another route of administration includes pulmonary administration. In addition, it can be desirable to administer a therapeutically effective amount of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In some aspects, the therapeutically effective amount of the pharmaceutical composition is delivered in a vesicle, such as liposomes (see, e.g., Langer, Science 249:1527-33, 1990 and Treat et al., in Liposomes in the Therapy of Infectious Disease and Cancer, Lopez Berestein and Fidler (eds.), Liss, N.Y., pp. 353-65, 1989).

In some aspects, a pharmaceutical composition described herein can be delivered in a controlled release system. For instance, in some aspects, a pump can be used (see, e.g., Langer, Science 249:1527-33, 1990; Sefton, Crit. Rev. Biomed. Eng. 14:201-40, 1987; Buchwald et al., Surgery 88:507-16, 1980; Saudek et al., N Engl. J Med. 321:574-79, 1989). In some aspects, polymeric materials can be used (see, e.g., Levy et al., Science 228:190-92, 1985; During et al., Ann. Neural. 25:351-56, 1989; Howard et al., J Neurosurg. 71:105-12, 1989). Other controlled release systems, such as those discussed by Langer (Science 249:1527-33, 1990), can also be used.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELS (BASF; Parsippany, NJ), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying, which yields a powder of the active ingredient plus any additional desired ingredient from a previously sterile-filtered solution thereof.

For administration by inhalation, the compounds are delivered in the form of an aerosol spray from a pressurized container or dispenser that contains a suitable propellant, e.g., a gas such as carbon dioxide, or a nebulizer. Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In some aspects, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

In some aspects, active compounds of the present disclosure can be formulated in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such a functional compound for the treatment of individuals. The pharmaceutical compositions can be included in a container, pack, or dispenser together with instructions for administration.

### III. Methods of the Disclosure

Some aspects of the present disclosure are directed to nucleic acid molecules that increase the expression of a target heterologous mRNA. As such, the compositions of the present disclosure can be used in methods to increase expression of a target in a cell, including a cell in a human subject in need of a treatment.

Some aspects of the present disclosure are directed to a method of expressing a target mRNA in a cell, comprising co-expressing the target mRNA and an influenza NS1 protein in the cell, wherein the target mRNA is not an influenza mRNA. In some aspects, the influenza NS1 protein is encoded by a first nucleic acid molecule and the target mRNA is encoded by a second nucleic acid molecule, e.g., as described herein.

Some aspects of the present disclosure are directed to method of treating a disease or condition in a subject in need thereof comprising administering to the subject composition disclosed herein, e.g., a polynucleotide or set of polynucleotides disclosed herein, a vector or a set of vectors disclosed herein, a lipid nanoparticle a cell disclosed herein, or a pharmaceutical composition disclosed herein. For example, a composition described herein can be administered to cells in culture, in vitro or ex vivo, or to human subjects, e.g., in vivo, to induce the selective and persistent expression of an encoded target heterologous mRNA in a cell (e.g., a tumor cell and/or an immune cell), which, in some aspects, can help treat a disease or disorder.

Accordingly, in some aspects, the present disclosure is directed to therapeutic methods using a composition described herein. In some aspects, disclosed herein is a method of expressing a target heterologous mRNA in a subject in need thereof, comprising administering to the subject a composition of the present disclosure.

In some aspects, a disease or disorder that can be treated with the present disclosure includes a cancer. In some aspects, the cancer comprises a squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, or any combination thereof.

In some aspects, a composition described herein is administered to a subject in need thereof at an amount sufficient to reduce tumor burden or cancer cell growth in vivo by at least about 5%, at least about 10%, at least about 20%, at least about 30%, at least about 40%, at least about 50%, at least about 60%, at least about 70%, at least about 80%, at least about 90%, or greater. In some aspects, the composition described herein is administered in an amount effective in increasing immune activity by at least about 5%, at least about 10%, at least about 20%, at least about 30%, at least about 40%, at least about 50%, at least about 60%, at least about 70%, at least about 80%, at least about 90%, or greater.

In some aspects, a disease or disorder that can be treated with the present disclosure comprises an autoimmune disease. As used herein, the term “autoimmune disease” refers to a disease caused by an inability of a host's immune system to distinguish foreign molecules from self-molecules, such that the host's immune system attacks and destroys the self-molecules. As used herein, “self-molecules” (e.g., protein or DNA) refer to a molecule that is derived from or is native to a host. As used herein, “foreign molecules” refer to molecules that are derived from another, and are of a non-native origin. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, Graves' disease, myasthenia gravis, pemphigus vulgaris, anti-glomerular basement membrane disease (Goodpasture syndrome), Hashimoto's thyroiditis, autoimmune hepatitis, and combinations thereof.

In some aspects, where the disease or disorder to be treated comprises an autoimmune disease, administering the composition (e.g., the polynucleotide, vector, lipid nanoparticle, or pharmaceutical composition described herein) to a subject can decrease immune activity, such as T cell activity, in the subject. For instance, as is apparent from the present disclosure, in some aspects, by encoding a target heterologous mRNA that is capable of decreasing immune cell function and/or promoting immune suppressor activity (e.g., promoting the development regulatory T cells), a polynucleotide described herein can be used to decrease immune activity. In some aspects, the immune activity is decreased by at least about 5%, by at least about 10%, by at least about 20%, by at least about 30%, by at least about 40%, by at least about 50%, by at least about 60%, by at least about 70%, by at least about 80%, by at least about 90% or more, compared to the immune activity of a reference subject (e.g., the subject prior to the administration of the composition or a corresponding subject that did not receive an administration of the composition).

### EXAMPLES

### Example 1

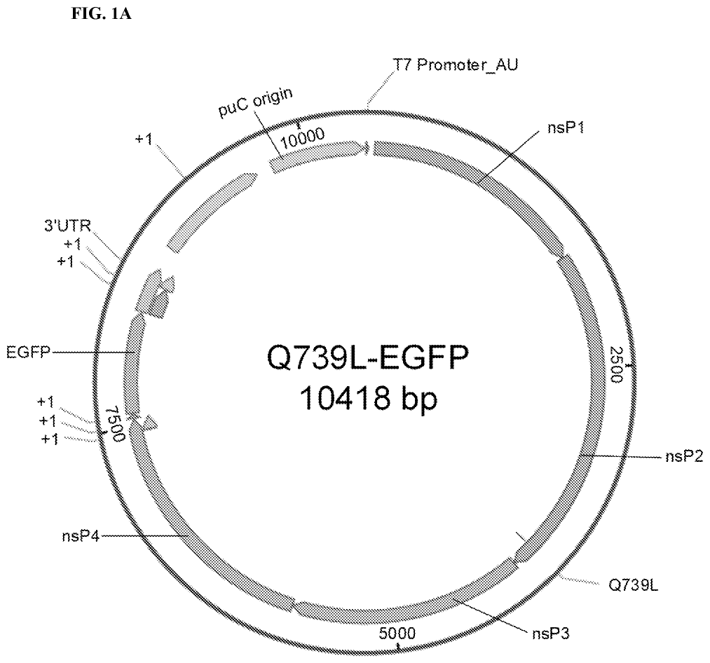

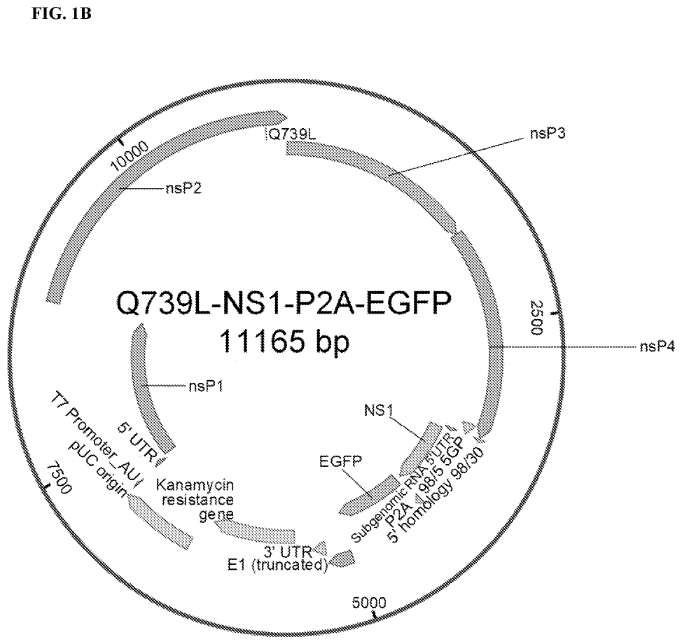

Replicon plasmids used in the present example included the following: Strand-EGFP (SEQ ID NO: 3; Table 6); Strand-mCherry (SEQ ID NO: 4; Table 6); non-cytopathic-EGFP (SEQ ID NO: 5; FIG. 1A; Table 6); Strand-NS1-H5N1 (SEQ ID NO: 2; Table 1); Strand-NS1-TX91 (SEQ ID NO: 1; Table 1); Strand-NS1-EGFP (SEQ ID NO: 6; Table 6); Strand-NS1-mCherry (SEQ ID NO: 7; Table 6); and non-cytopathic-NS1-EGFP (SEQ ID NO: 8; FIG. 1B; Table 6).

### Methods

### Template Preparation

For replicon RNA, the VEE replicon vector containing the EGFP-encoding mRNA payload was prepared by a suitable plasmid preparation method. The plasmid was further linearized by BspQI treatment. Briefly, 5 μg of replicon plasmid DNA was treated with BspQI in NEB3.1 buffer for 3 hr at 50° C. The enzyme was heat inactivated at 80° C. for 20 minutes, and the samples proceeded to a DNA cleanup step.

TABLE 3

| Component | Volume | Concentration | |
|---|---|---|---|
| DNA template | | 1 | ug |
| Cutsmart buffer/NEB 3.1 | 5 | 1x | |
| BspQI/IsceI | 1 | 5 | units |

| | |
|---|---|
| Water | 44 |

For mCherry containing replicon plasmids (Strand-mCherry and Strand-NS1-mCherry) as well as only NS1 containing plasmids (Strand-NS1-H5N1 and Strand-NS1-TX91), ISceI was used to digest the replicon template. The reaction included Cutsmart buffer at 37° C. for 3 hrs.

modRNA Template Generation

For modified RNA (modRNA) templates, the DNA was generated by PCR using a replicon plasmid with forward primer containing T7 promoter and subgenomic promoter and a reverse primer in the 3′-UTR with a stretch of 120 amino acids.

TABLE 4A
PCR setup

| | | | FINAL | |
|---|---|---|---|---|
| COMPONENT | 25 μl REACTION | | CONCENTRATION | |
| 10 μM Forward Primer | 1.25 | μl | 0.5 | μM |
| 10 μM Reverse Primer | 1.25 | μl | 0.5 | μM |

| | | | |
|---|---|---|---|
| Template DNA | variable | 10 | ng |

| | | |
|---|---|---|
| 2X Q5 Hot | 12.5 | μl |
| Start Master mix | | |
| Nuclease-Free Water | to 25 | μl |

TABLE 4B
PCR Cycling Conditions

| | | |
|---|---|---|
| STEP | TEMP | TIME |

| | | | | |
|---|---|---|---|---|
| Initial Denaturation | 98° | C. | 30 | seconds |
| 30 Cycles | 98° | C. | 10 | seconds |
| | 72° | C. | 4 | minutes |
| Final Extension | 72° | C. | 4 | minutes |

| | | |
|---|---|---|
| Hold | 4-10° | C. |

### DpnI Digestion

Plasmid DNA (template) in the PCR reaction was digested by DpnI. Add 1 uL DpnI per ug of initial plasmid to PCR sample and incubated for 1 hr at 37° C.

### DNA Cleanup

PCR-amplified DNA samples were cleaned using the NEB PCR cleanup kit, according to the manufacture's protocol, and DNA was eluted in 20 μL water.

PCR (modRNA template) and BspQI-treated replicon DNA (repRNA template) were checked on the pre-cast gels to confirm the purity (PCR) and integrity (replicon template). 1 μg of purified template was employed in each 20 μL IVT reaction, below.

Up to 20 ng total DNA was loaded on the 1.2% DNA gels and run at 275V for 7-10 minutes.

### In Vitro Transcription

NEB HiScribe High yield T7 kit was used for RNA production. For modified RNA (modRNA) synthesis, he UTP component of the kit was replaced by N1-methylpseudouridine-5′-triphosphate.

When ready for the IVT reaction, the necessary kit components were thawed on ice, mixed and pulse-spinned in microfuge to collect solutions to the bottom of tubes. Samples were kept on ice, and the enzyme was not vortexed.

### Co-Transcriptional Capping Method

For production using cap analog replicon plasmids and modRNA templates, the reaction was assembled at room temperature in the following order:

| Component | Volume | | Final Conc. |
|---|---|---|---|
| Nuclease-free water | X | μl | |
| 10X Reaction Buffer (NEB | 10 | μl | 1X |
| ATP (100 mM) | 10 | μl | 10 mM final |
| GTP (100 mM) | 10 | μl | 10 mM final |
| UTP or N1-methylpseudoUTP (100 mM) | 10 | μl | 10 mM final |
| CTP (100 mM) or 5-methylCTP (100 mm) | 10 | μl | 10 mM final |
| Cap Analog | 5 | μl | |
| Template DNA | X | μl | 1 μg |
| T7 RNA Polymerase Mix | 10 | μl | |
| SUPERase Inh. | 5 | μl | |
| Total reaction volume | 100 | μl | |

Samples were then mixed thoroughly, pulse-spun in microfuge, and incubated at 37° C. for 3 hours in a thermomixer at 400 rpm. A 1 uL aliquot was reserved for quality control.

### DNase Treatment

Turbo DNase enzyme was used to digest template DNA. 10× buffer was not added as the enzyme is active in IVT reactions. The reaction was diluted to 200 uL with nuclease free water.

20 μL enzyme (2U/μL) was used per 100 μL IVT reaction and incubated for 60 minutes at 37° C. Following the reaction, the RNA was purified using Monarch RNA cleanup kit. A 1 μL aliquot of RNA was kept for quality control.

### RNA Quality Control

The concentration of the sample was checked on Nanodrop and quality control on gel with RNA from all intermediate steps. Up to 200 ng RNA was run on a 1.2% RNA gel. 1 uL of Lonza RNA ladder was used as a size marker. RNA was denatured by adding 50% formaldehyde sample buffer at 65° C. for 5 minutes, and the samples were immediately kept on ice for at least one minute.

Up to 5 uL total sample was loaded on the gel and visualized. RNA integrity can also be checked by running on a fragment analyzer.

Prepare Conventional TT3 LNPs Formulations with T-Junction

The lipid materials were each weighed out and dissolved in ethanol. The ethanol phase was prepared by mixing all the lipid materials according to composition ratio in the form below. The aqueous phase was prepared by diluting the silica column purified repRNA/modRNA with 20 mM Citrate Buffer (pH 4.0), 300 mM NaCl and water so that the final composition of the salt was 10 mM citrate buffer (pH 4.0, 150 mM NaCl). The conventional TT3 LNPs were afforded by mixing the ethanol phase and aqueous phase of the LNPs through T-junction mixing at the flow rate ratio of 3:1 (aqueous phase: ethanol phase).

TABLE 5
TT3 LNP Composition (mg)

| | |
|---|---|
| mRNA | 1.0 |
| TT3 | 10.0 |
| DOPE | 8.0 |
| Cholesterol | 5.6 |
| DMG-PEG-2k | 0.7 |

Preparation of Post-PEG Micelles TT3 LNPs Formulations with T-Junction

The lipid materials were each weighed out and dissolved in ethanol. The ethanol phase was prepared by mixing all the lipid materials except from DMG-PEG-2K, according to composition ratio in the form above. The aqueous phase was prepared by diluting the silica column purified repRNA/modRNA with 20 mM Citrate Buffer (pH 4.0), 300 mM NaCl and water so that the final composition of the salt was 10 mM citrate buffer (pH 4.0, 150 mM NaCl). PEG micelle phase was prepared by adding the corresponding volume of DMG-PEG-2K into TBS buffer and mixing thoroughly via vortex. Finally, the post-PEG micelles TT3 LNPs were afforded by first mixing the ethanol phase and aqueous phase of the LNPs through a T-junction mixing at the flow rate ratio of 3:1 (aqueous phase: ethanol phase), and followed by an immediate in-line dilution with the PEG micelle phase viaT-junction mixing at the flow rate ratio of 1:1 (LNP phase:PEG phase). The final lipid composition of post-PEG micelle TT3 LNP is described in Table B.

TABLE A

| | Weight Ratio |
|---|---|
| TT3 LNP Composition Component | |
| mRNA | 1.0 |
| TT3 | 10.0 |
| DOPE | 8.0 |
| Cholesterol | 5.6 |
| PEG-DMG-2k Micelle | |
| mRNA | 1.0 |
| DMG-PEG-2K | 4.2 |

TABLE B

| TT3 LNP Composition Component | Weight Ratio |
|---|---|
| mRNA | 1.0 |
| TT3 | 10.0 |
| DOPE | 8.0 |
| Cholesterol | 5.6 |
| DMG-PEG-2K | 4.2 |

### Buffer Exchange and Freeze/Thaw of TT3 LNPs

The afforded TT3 LNPs were transferred to the dialysis cassettes and dialyze in TBS buffer for 2 hours. Then the TT3 LNPs were concentrated via tangential flow filtration. Subsequently, 40% sucrose (W/V) in TBS stock solution was added into all the prepared TT3 LNPs to make a final solution of TT3 LNPs in 10% sucrose. The final RNA concentrations of LNPs were measured by dissociating the LNPs with 2% TE+Triton and further detected with Qubit assay. TT3 LNPs were aliquot into 50 l/tube aliquots and put the at −80° C. for freezing. Before treating cells with LNPs, TT3 LNPs were thawed at room temperature.

### Lipid Transfection

For the cell lines, 50 ng TT3:RNA was added to respective wells. For some experiments, Lipofectamine MessengerMax (Thermo Fisher) was used to deliver payloads according to manufacturer's recommendations.

### FACS Sample Preparation

Zombie NIR staining buffer was prepared by diluting the 100X dye stock in PBS. Cells were washed in PBS and transferred to a deep well 96-well plate and centrifuged at 500g, 10 minutes for suspension cells. For adherent cells, cells are trypsinized for 5 minutes followed by centrifugation at 500g, 10 minutes. Cells are resuspended in PBS and transferred to 96 V-bottom plate, followed by live/dead staining using 100 μL of PBS containing the Zombie NIR dye for 10 minutes at RT in the dark. The reaction is stopped by adding 200 μL of FACS buffer (contains BSA to quench the dye) and centrifuged again and resuspended finally in 200 uL FACS buffer. The cells are analyzed on the flow cytometer for reporter (GFP or mCherry) signal and viability.

### Results

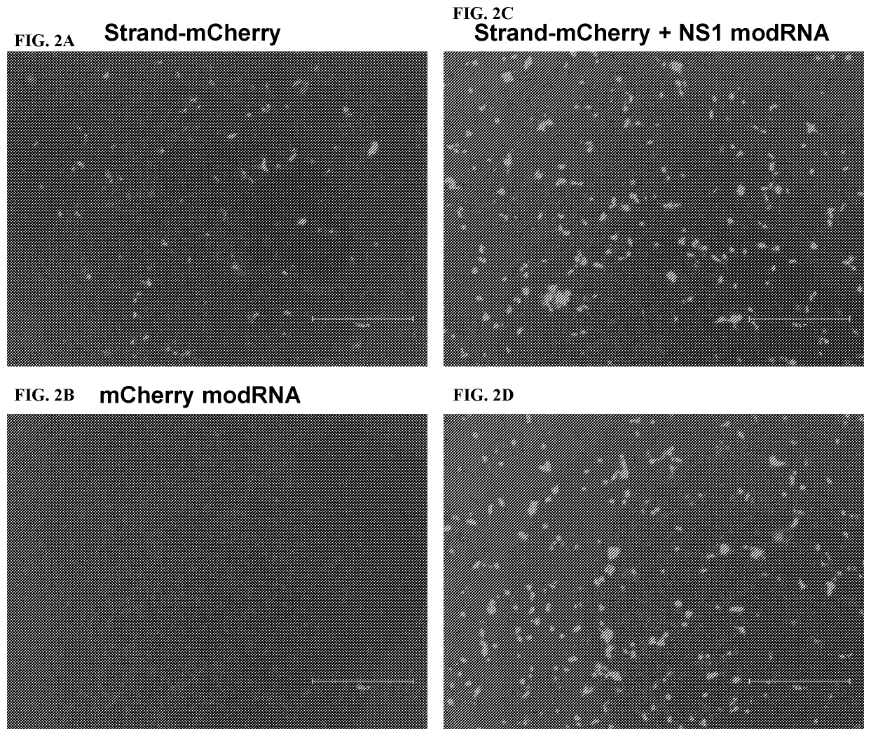

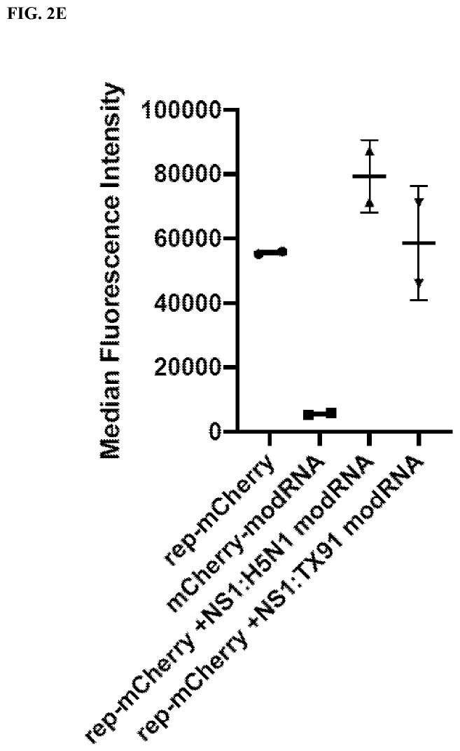

Tandem transfection of NS1 with repRNA improved replicon expression in the 4T1 mouse tumor cell line. 4T1 cells were transfected with Strand-mCherry, mCherry modRNA, NS1 modRNA mRNAs using Lipofectamine MessengerMax. In this tandem transfection, NS1 modRNA was transfected 6 hrs prior to transfecting the repRNA. This provides cells the opportunity to provide NS1 protein before the cells experience the replicon. Twenty-four hours following transfection, cells were visualized by microscopy as well as processed by Flow cytometry to look at the quantitative signal (FIGS. 2A-2D). Tandem transfection of NS1 from both H5N1 and TX91 with repRNA improved replicon expression (FIG. 2E).

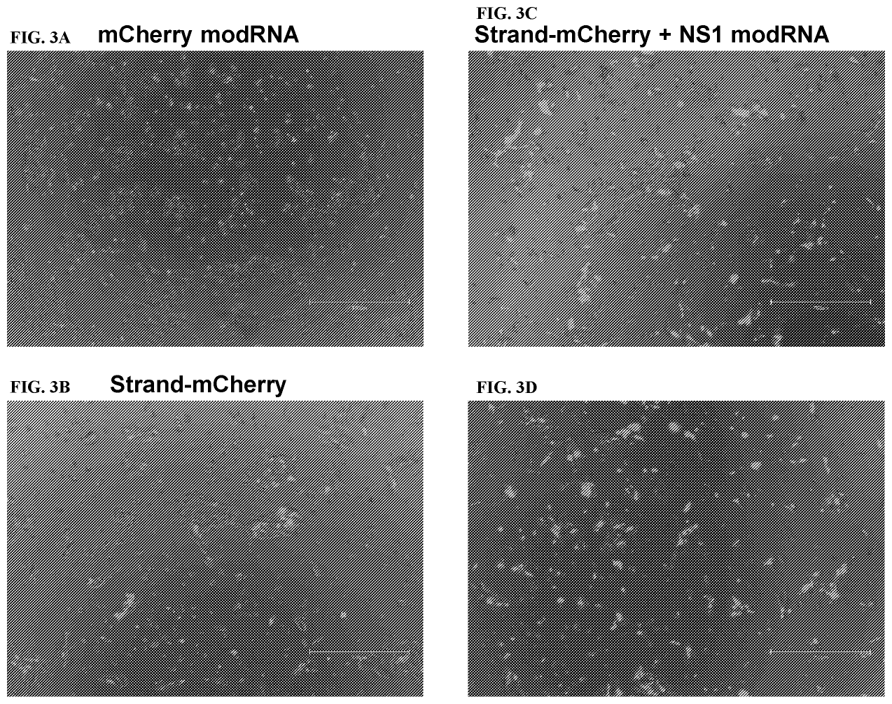

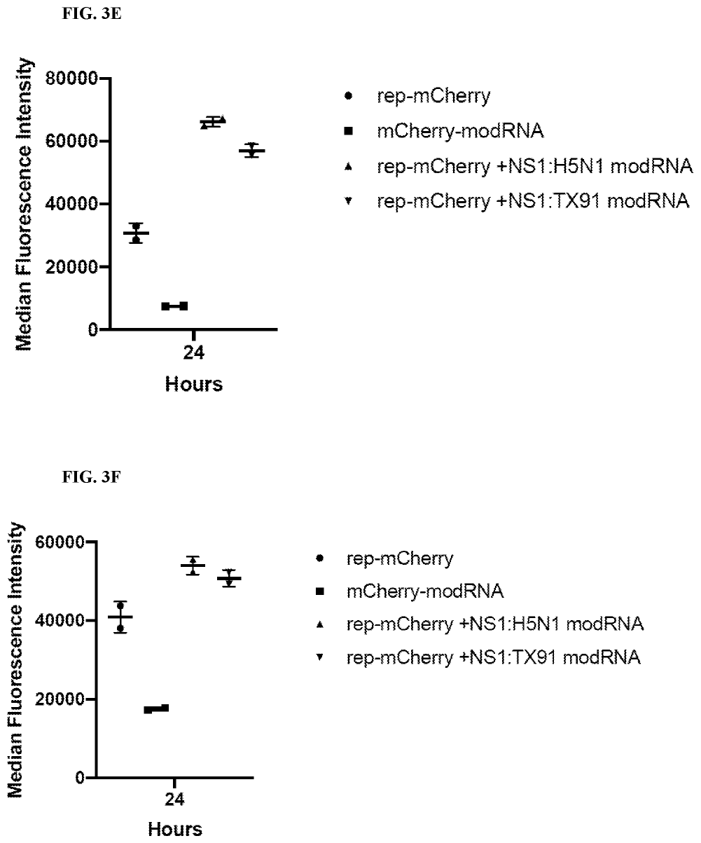

Next, 4T1 cells were transfected with Strand-mCherry, mCherry modRNA, NS1 modRNA mRNAs using TT3 lipid nanoparticle. In this co-transfection, NS1 modRNA was transfected together with the repRNA. Twenty-four hours following transfection, cells were visualized by microscopy as well as processed by Flow cytometry to look at the quantitative signal (FIGS. 3A-3F). Tandem NS1 expression (FIG. 3E) and NS1 cotransfection (FIG. 3F) mediated higher MFI from repRNA in 4T1 cells.

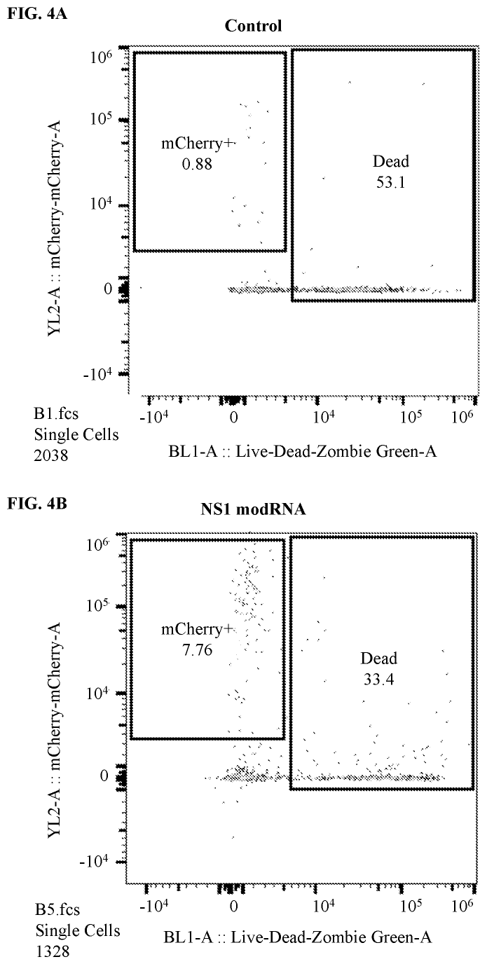

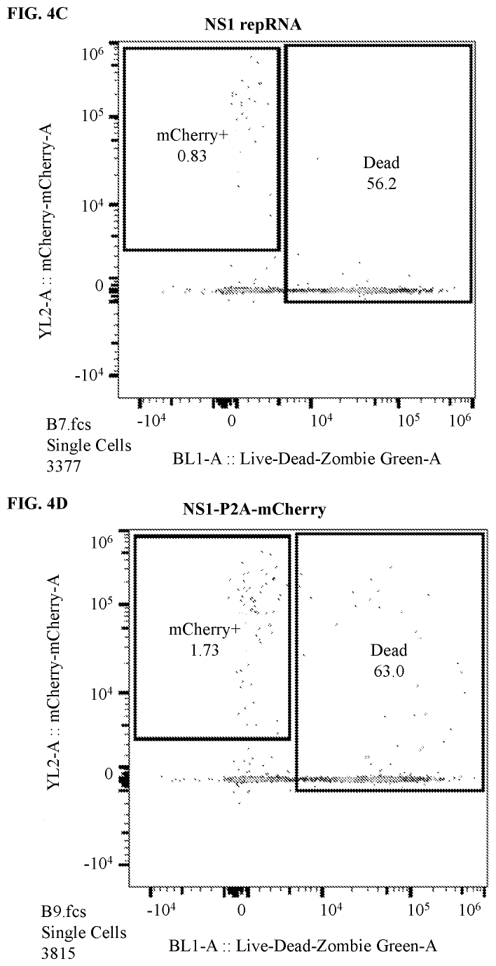

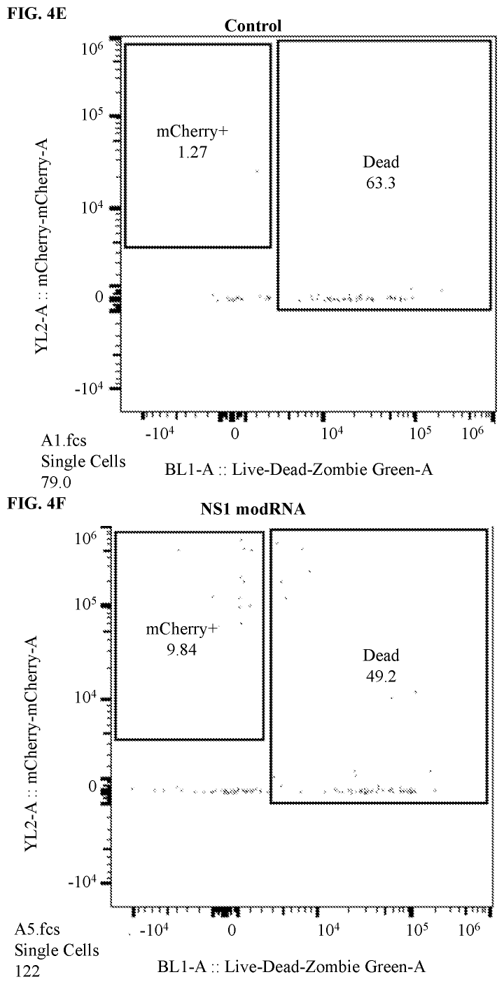

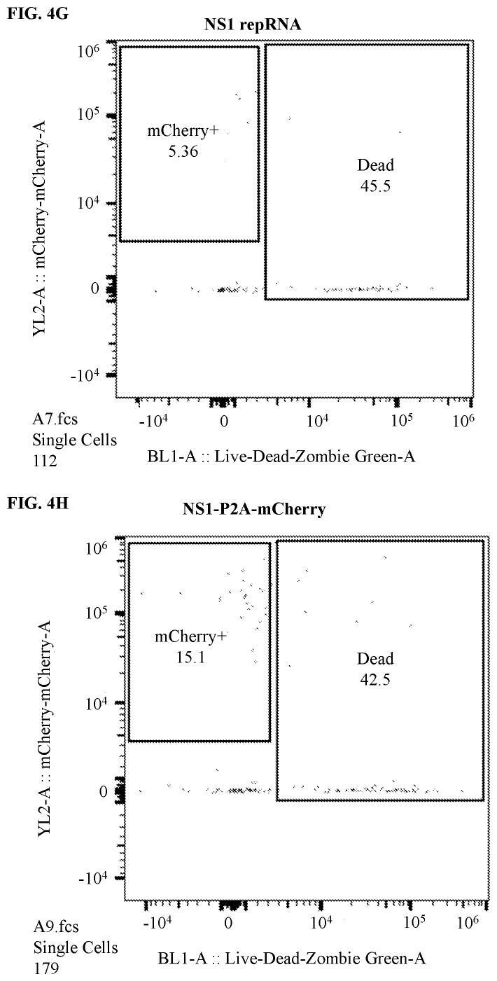

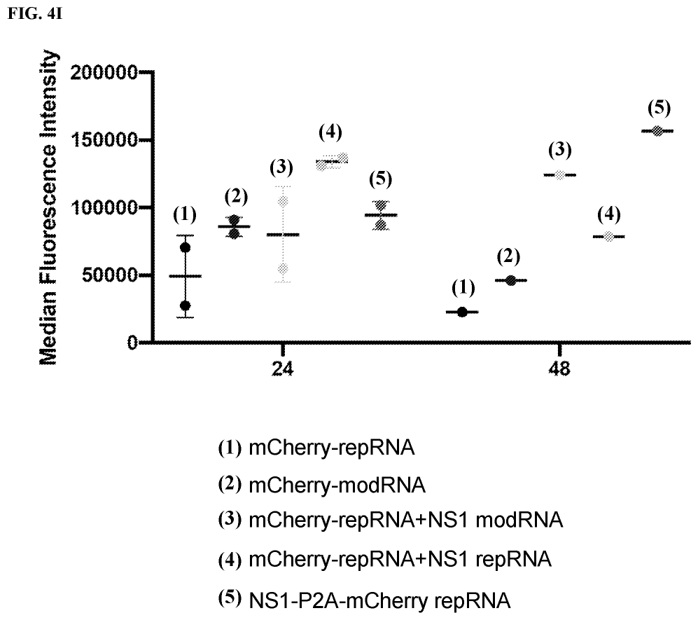

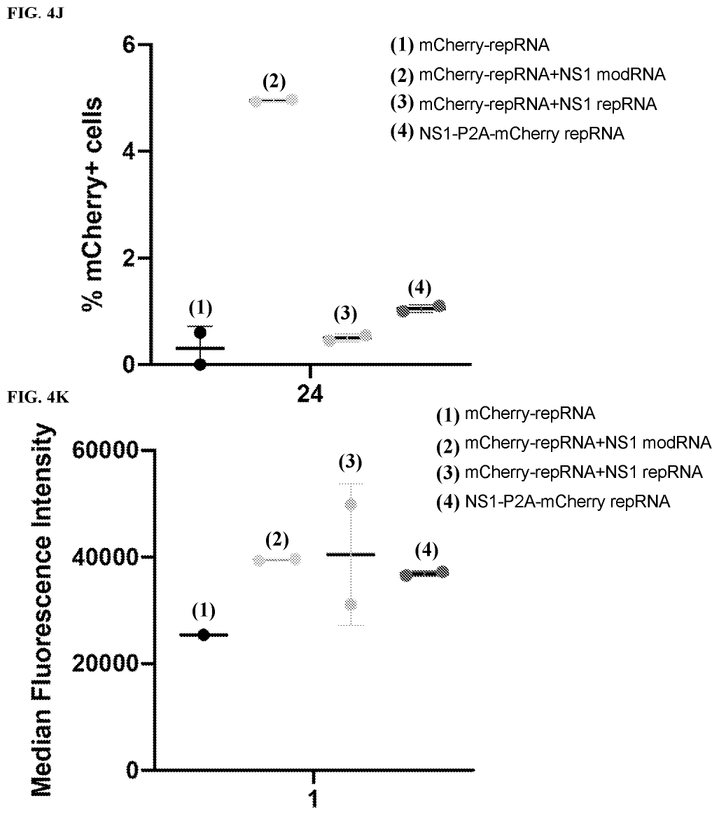

Co-transfection of NS1 with repRNA improves replicon expression in a variety of human cancer cell lines. Hcc38 tumor cells were transfected with NS1 modRNA (FIGS. 4B and 4F), NS1 repRNA (FIGS. 4C and 4G), or NS1-P2A-mCherry mRNA (FIGS. 4D and 4H) using Lipofectamine MessengerMax. In this experiment, NS1 was provided either as a modRNA (NS1 modRNA; FIGS. 4B and 4F) or as a separate (NS1 repRNA; FIGS. 4C and 4G) or a bi-cistronic repRNA vector (NS1-P2A-mCherry; FIGS. 4D and 4H). Twenty-four (FIGS. 4A-4D) or forty-eight (FIGS. 4E-4H) hours following transfection, cells were visualized by microscopy as well as processed by Flow cytometry to look at the quantitative signal. Similarly, median fluorescence intensity (MFI) was observed to increase with NS1 expression in Scc9 HNSCCs (FIG. 4I) and FaDu HNSCC cells (FIGS. 4J-4K).

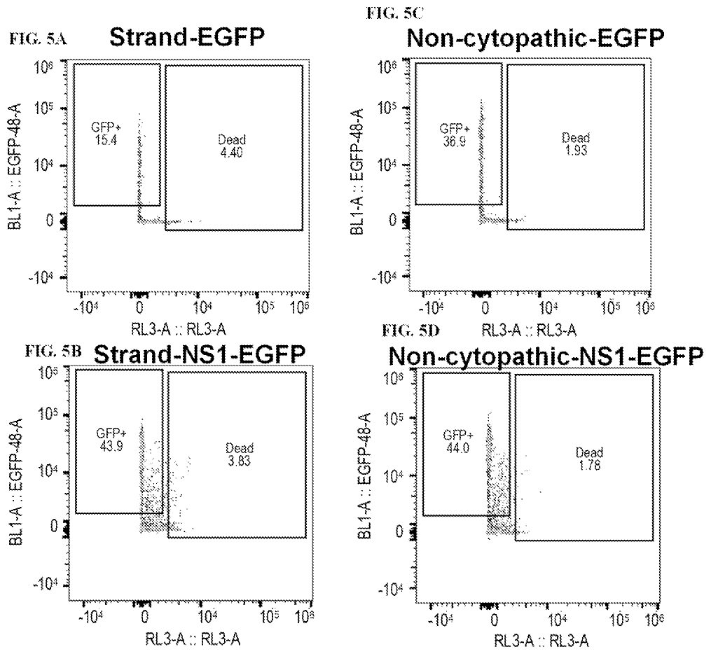

Additive improvements were observed when combining vector engineering with NS1. BT20 cancer cells were transfected using TT3 LNP using EGFP encoding replicon vector containing either the original (Strand) backbone (FIGS. 5A-5B) or one containing a Q739L mutation (non-cytopathic; FIGS. 5C-5D) and the cells were analyzed using Flow cytometry to quantify the reporter signal. An additive improvement was readily apparent when combining NS1 in the Q739L vector (FIGS. 5A-5F).

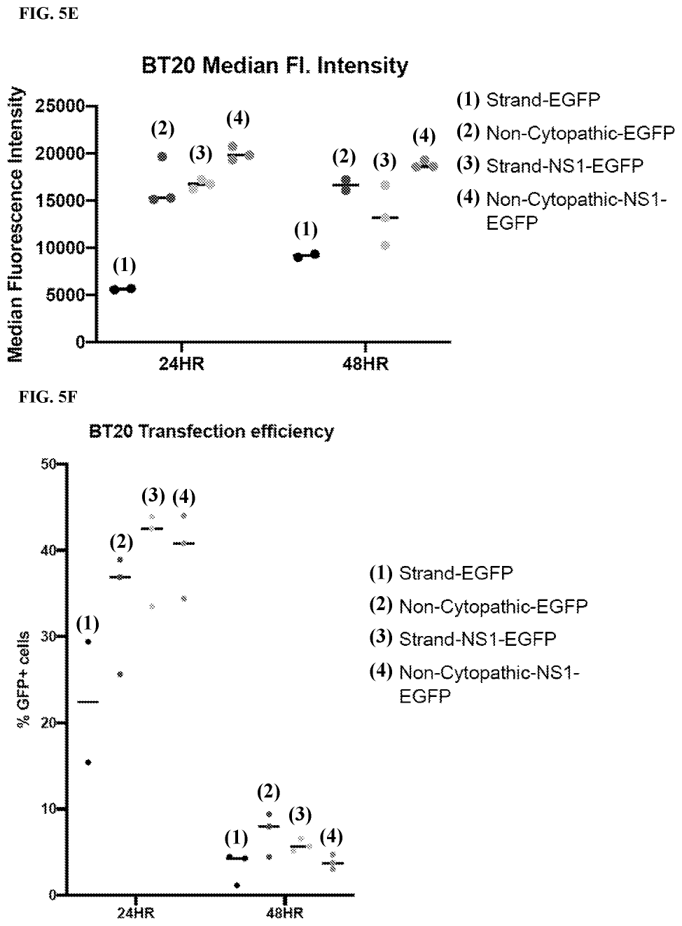

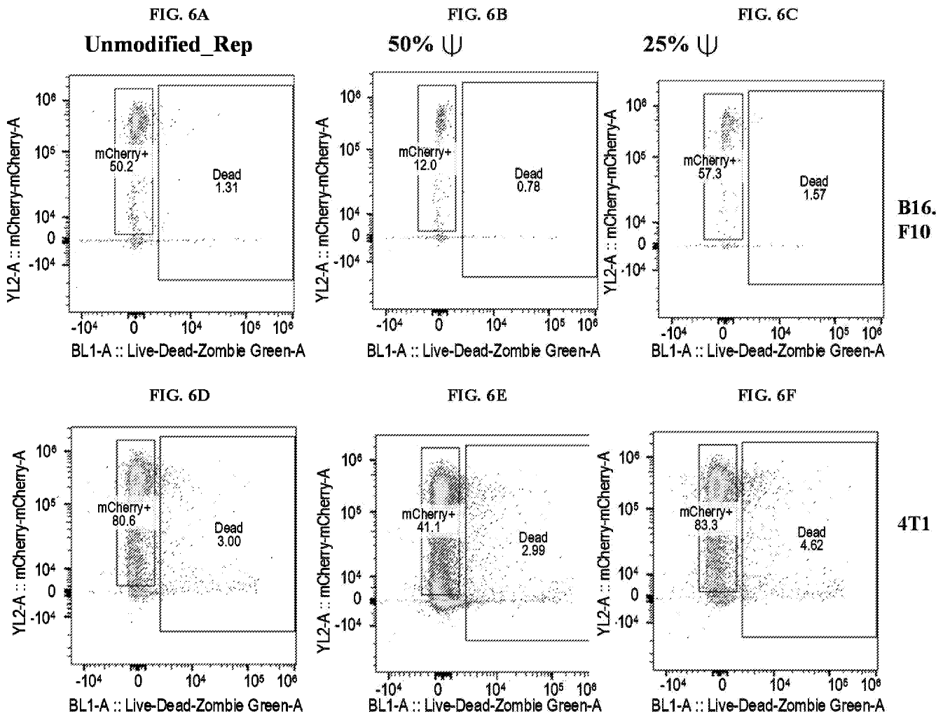

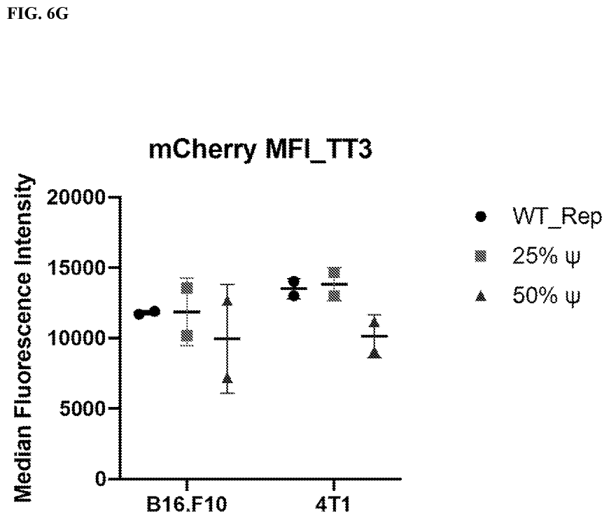

Next, B16.F10 (FIGS. 6A-6C) or 4T1 (FIGS. 6D-6F) cells were electroporated with replicon made either using unmodified rNTPs (Unmodified Rep; FIGS. 6A and 6D) or using a ratio of 1:1 (50%; FIGS. 6B and 6E) or 1:3 (25%; FIGS. 6C and 6F) UTP to N1-methyl-pseudoUTP (denoted by psi). While 1:1 ratio leads to significant reduction in payload expression, 1:3 ratio maintains the expression as well as signal intensity (FIG. 6G).

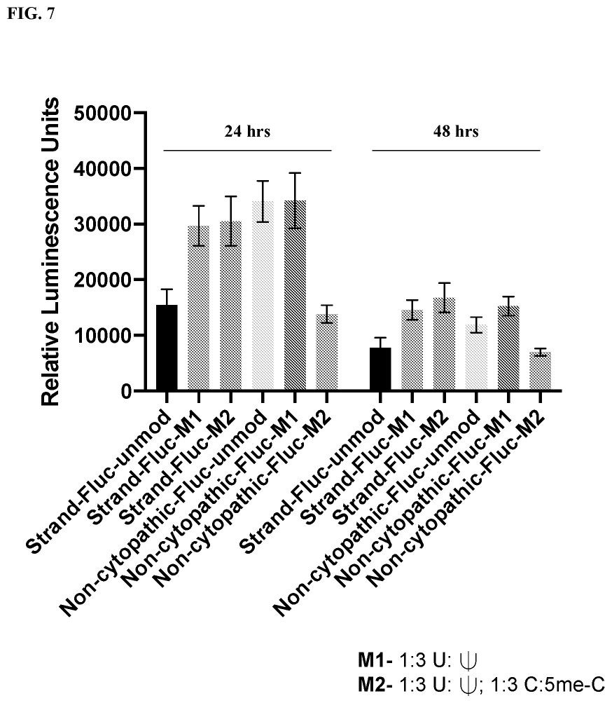

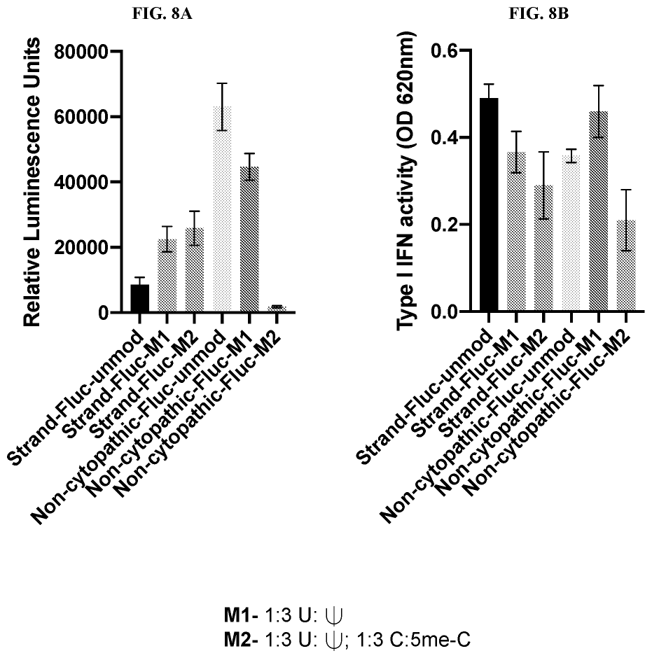

Additive effects were observed for the combination of vector engineering and the use of modified rNTPs in replicon driven payload expression (FIG. 7). Firefly luciferase (Fluc) encoded replicons were made either using unmodified rNTPs (unmod) or using a 1:3 ratio of certain NTPs-U: Li (M1) or U: Li; 1:3 C:5me-C(M2), where C refers to Cytidine and 5me-C refers to 5-methyl-cytidine. Luminescence was measured at 24 as 48 hrs post transfection via electroporation in 4T1 cells (FIG. 7). These Fluc replicons were next transfected in an interferon inducible cell line, B16-ISG (Invivogen) and payload expression was measured in Luminescence units (FIG. 8A) while Type I IFN activity was measured using the SEAP reporter assay using colorimetry (FIG. 8B). Singly (M1) or doubly modified (M2) replicons showed improvement in payload expression as well as reduced Type I IFN activity.

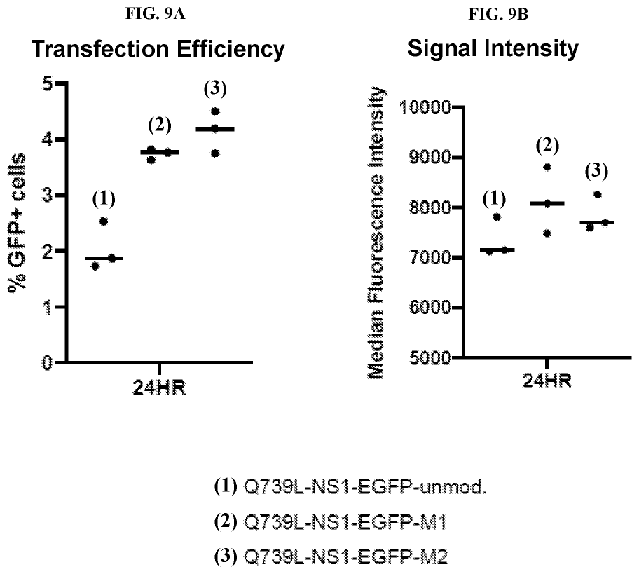

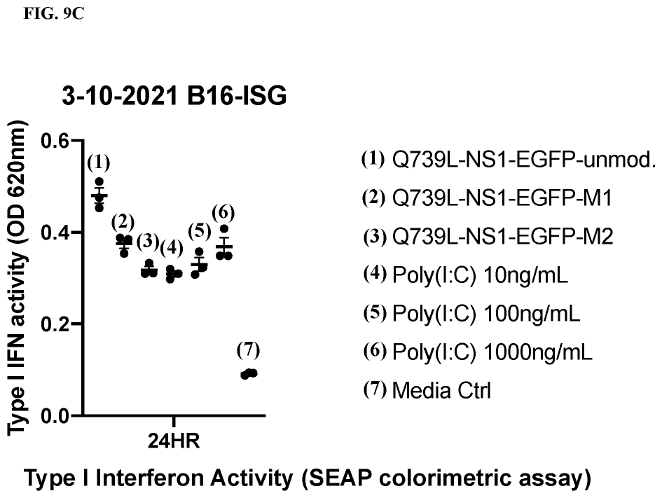

The use of modified rNTPs in replicon driven payload expression improved expression and reduced IFN activation (FIGS. 9A-9C). B16-ISG cells were transfected with a Q739L replicon expressing NS1-EGFP (P2A linker) made either using unmodified or singly (M1) or doubly modified rNTPs, as described above. Doubly modified replicons improve payload expression (FIG. 9A), signal intensity (FIG. 9B), and reduced Type I IFN activity (FIG. 9C) as compared to Poly (I:C) treated cells. Poly (I:C) is a dsRNA analog and a strong Type I IFN agonist.

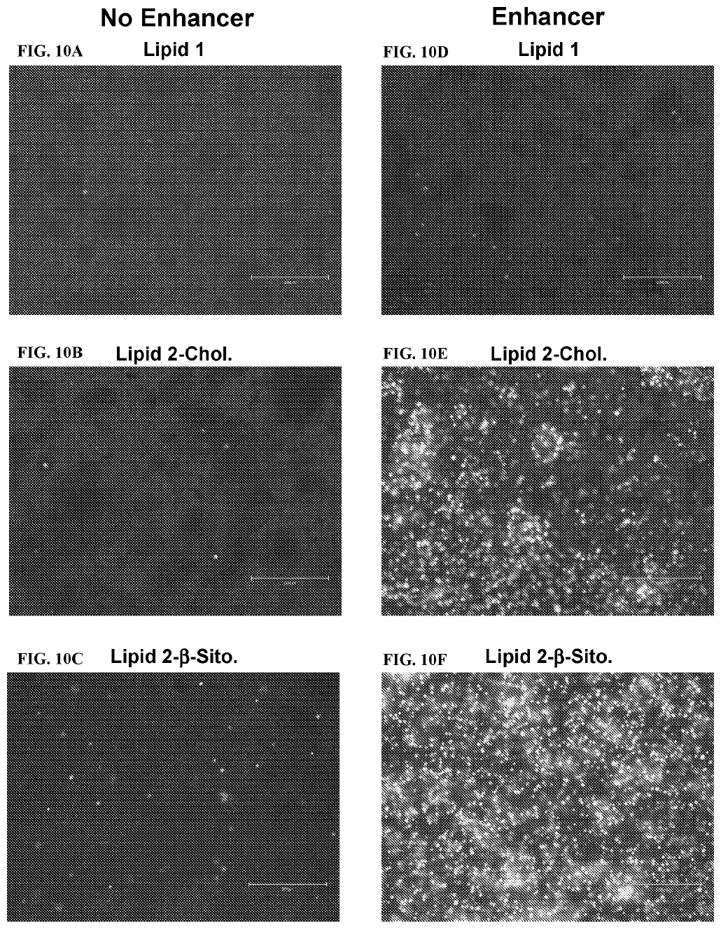

LNP delivery in T cells activated for 2 days with Anti-CD3/CD28/CD2 cocktail with high dose IL-2 was improved by addition of a recombinant protein (Enhancer) to the media, leading to robust signal in cells transfected with lipid 2-cholesterol (FIG. 10E) or lipid 2-b-sitosterol (FIG. 10F) as compared to cells transfected with Lipid 1 and cells not exposed to Enhancer (FIGS. 10A-10D).

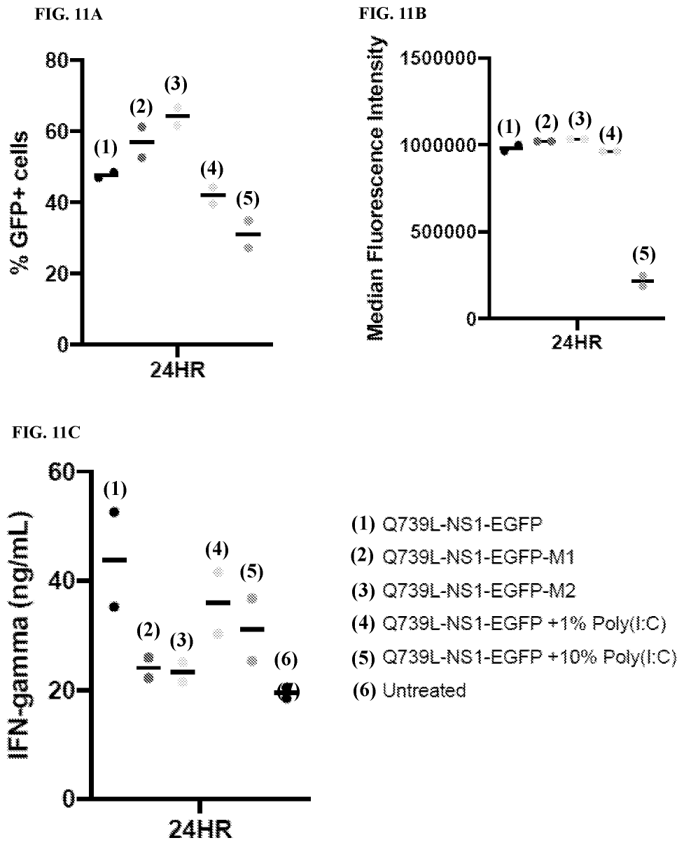

Use of modified rNTPs in replicon driven payload expression improved expression and reduced IFN activation. Primary human T cells were activated using IL-2 and anti-CD3/CD28/CD2 for 2 days post thaw and transfected using the Q739L replicon driving NS1-EGFP, as used above, and made using unmodified or singly (M1) or doubly modified (M2) mRNAs. As an addition, unmodified vectors were spiked with 1 or 10% Poly (I:C), the dsRNA analog, and co-encapsulated in the lipid. Twenty-four hours post transfection, the cells were analyzed by flow cytometry to quantify GFP signal, and the cell supernatants were used to analyze pro-inflammatory cytokines, e.g., IFN-gamma. The M2 replicons show the highest level of expression (FIGS. 11A-11B) while maintaining the lowest amount of IFN-gamma secretion (FIG. 11C).

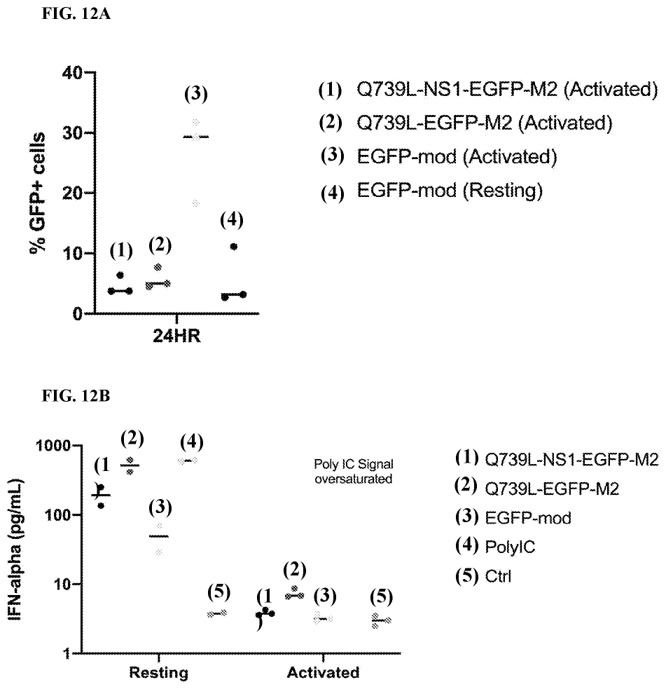

Human PBMCs were isolated from three healthy donors and either grown with low dose IL-2 (Resting) or with high dose of IL-2 in presence of Anti-CD3/CD28/CD2 cocktail (Activated) for 2 days followed by mRNA:lipid delivery with M2 modified Q739L replicons driving NS1-EGFP or EGFP or with conventional EGFP mRNA (EGFP-mod). Twenty-four hours post-transfection, the cells were stained with a Live-dead viability stain and analyzed for GFP signal (FIG. 12A). The supernatants were collected for cytokine profiling and were subjected to ELISA for IFN-alpha detection. NS1 encoding replicons reduced IFN-alpha activation in both resting and activated states (FIG. 12B).

### Sequences

TABLE 6
Sequences

Strand-EGFP (SEQ ID NO: 3)

| | |
|---|---|
| 1 | gctcttctaa gTAATACGAC TCACTATAAT GGGCGGCGCA TGAGAGAAGC CCAGACCAAT |
| 61 | TACCTACCCA AAATGGAGAA AGTTCACGTT GACATCGAGG AAGACAGCCC ATTCCTCAGA |
| 121 | GCTTTGCAGC GGAGCTTCCC GCAGTTTGAG GTAGAAGCCA AGCAGGTCAC TGATAATGAC |
| 181 | CATGCTAATG CCAGAGCGTT TTCGCATCTG GCTTCAAAAC TGATCGAAAC GGAGGTGGAC |
| 241 | CCATCCGACA CGATCCTTGA CATTGGAAGT GCGCCCGCCC GCAGAATGTA TTCTAAGCAC |
| 301 | AAGTATCATT GTATCTGTCC GATGAGATGT GCGGAAGATC CGGACAGATT GTATAAGTAT |
| 361 | GCAACTAAGC TGAAGAAAAA CTGTAAGGAA ATAACTGATA AGGAATTGGA CAAGAAAATG |
| 421 | AAGGAGCTCG CCGCCGTCAT GAGCGACCCT GACCTGGAAA CTGAGACTAT GTGCCTCCAC |
| 481 | GACGACGAGT CGTGTCGCTA CGAAGGGCAA GTCGCTGTTT ACCAGGATGT ATACGCGGTT |
| 541 | GACGGACCGA CAAGTCTCTA TCACCAAGCC AATAAGGGAG TTAGAGTCGC CTACTGGATA |
| 601 | GGCTTTGACA CCACCCCTTT TATGTTTAAG AACTTGGCTG GAGCATATCC ATCATACTCT |
| 661 | ACCAACTGGG CCGACGAAAC CGTGTTAACG GCTCGTAACA TAGGCCTATG CAGCTCTGAC |
| 721 | GTTATGGAGC GGTCACGTAG AGGGATGTCC ATTCTTAGAA AGAAGTATTT GAAACCATCC |
| 781 | AACAATGTTC TATTCTCTGT TGGCTCGACC ATCTACCACG AGAAGAGGGA CTTACTGAGG |
| 841 | AGCTGGCACC TGCCGTCTGT ATTTCACTTA CGTGGCAAGC AAAATTACAC ATGTCGGTGT |
| 901 | GAGACTATAG TTAGTTGCGA CGGGTACGTC GTTAAAAGAA TAGCTATCAG TCCAGGCCTG |
| 961 | TATGGGAAGC CTTCAGGCTA TGCTGCTACG ATGCACCGCG AGGGATTCTT GTGCTGCAAA |
| 1021 | GTGACAGACA CATTGAACGG GGAGAGGGTC TCTTTTCCCG TGTGCACGTA TGTGCCAGCT |
| 1081 | ACATTGTGTG ACCAAATGAC TGGCATACTG GCAACAGATG TCAGTGCGGA CGACGCGCAA |
| 1141 | AAACTGCTGG TTGGGCTCAA CCAGCGTATA GTCGTCAACG GTCGCACCCA GAGAAACACC |
| 1201 | AATACCATGA AAAATTACCT TTTGCCCGTA GTGGCCCAGG CATTTGCTAG GTGGGCAAAG |
| 1261 | GAATATAAGG AAGATCAAGA AGATGAAAGG CCACTAGGAC TACGAGATAG ACAGTTAGTC |
| 1321 | ATGGGGTGTT GTTGGGCTTT TAGAAGGCAC AAGATAACAT CTATTTATAA GCGCCCGGAT |
| 1381 | ACCCAAACCA TCATCAAAGT GAACAGCGAT TTCCACTCAT TCGTGCTGCC CAGGATAGGC |
| 1441 | AGTAACACAT TGGAGATCGG GCTGAGAACA AGAATCAGGA AAATGTTAGA GGAGCACAAG |
| 1501 | GAGCCGTCAC CTCTCATTAC CGCCGAGGAC GTACAAGAAG CTAAGTGCGC AGCCGATGAG |
| 1561 | GCTAAGGAGG TGCGTGAAGC CGAGGAGTTG CGCGCAGCTC TACCACCTTT GGCAGCTGAT |
| 1621 | GTTGAGGAGC CCACTCTGGA AGCCGATGTC GACTTGATGT TACAAGAGGC TGGGGCCGGC |
| 1681 | TCAGTGGAGA CACCTCGTGG CTTGATAAAG GTTACCAGCT ACGATGGCGA GGACAAGATC |
| 1741 | GGCTCTTACG CTGTGCTTTC TCCGCAGGCT GTACTCAAGA GTGAAAAATT ATCTTGCATC |
| 1801 | CACCCTCTCG CTGAACAAGT CATAGTGATA ACACACTCTG GCCGAAAAGG GCGTTATGCC |
| 1861 | GTGGAACCAT ACCATGGTAA AGTAGTGGTG CCAGAGGGAC ATGCAATACC CGTCCAGGAC |
| 1921 | TTTCAAGCTC TGAGTGAAAG TGCCACCATT GTGTACAACG AACGTGAGTT CGTAAACAGG |
| 1981 | TACCTGCACC ATATTGCCAC ACATGGAGGA GCGCTGAACA CTGATGAAGA ATATTACAAA |
| 2041 | ACTGTCAAGC CCAGCGAGCA CGACGGCGAA TACCTGTACG ACATCGACAG GAAACAGTGC |
| 2101 | GTCAAGAAAG AACTAGTCAC TGGGCTAGGG CTCACAGGCG AGCTGGTGGA TCCTCCCTTC |
| 2161 | CATGAATTCG CCTACGAGAG TCTGAGAACA CGACCAGCCG CTCCTTACCA AGTACCAACC |
| 2221 | ATAGGGGTGT ATGGCGTGCC AGGATCAGGC AAGTCTGGCA TCATTAAAAG CGCAGTCACC |
| 2281 | AAAAAAGATC TAGTGGTGAG CGCCAAGAAA GAAAACTGTG CAGAAATTAT AAGGGACGTC |
| 2341 | AAGAAAATGA AAGGGCTGGA CGTCAATGCC AGAACTGTGG ACTCAGTGCT CTTGAATGGA |
| 2401 | TGCAAACACC CCGTAGAGAC CCTGTATATT GACGAAGCTT TTGCTTGTCA TGCAGGTACT |
| 2461 | CTCAGAGCGC TCATAGCCAT TATAAGACCT AAAAAGGCAG TGCTCTGCGG GGATCCCAAA |
| 2521 | CAGTGCGGTT TTTTTAACAT GATGTGCCTG AAAGTGCATT TTAACCACGA GATTTGCACA |
| 2581 | CAAGTCTTCC ACAAAAGCAT CTCTCGCCGT TGCACTAAAT CTGTGACTTC GGTCGTCTCA |
| 2641 | ACCTTGTTTT ACGACAAAAA AATGAGAACG ACGAATCCGA AAGAGACTAA GATTGTGATT |
| 2701 | GACACTACCG GCAGTACCAA ACCTAAGCAG GACGATCTCA TTCTCACTTG TTTCAGAGGG |
| 2761 | TGGGTGAAGC AGTTGCAAAT AGATTACAAA GGCAACGAAA TAATGACGGC AGCTGCCTCT |
| 2821 | CAAGGGCTGA CCCGTAAAGG TGTGTATGCC GTTCGGTACA AGGTGAATGA AAATCCTCTG |
| 2881 | TACGCACCCA CCTCAGAACA TGTGAACGTC CTACTGACCC GCACGGAGGA CCGCATCGTG |
| 2941 | TGGAAAACAC TAGCCGGCGA CCCATGGATA AAAACACTGA CTGCCAAGTA CCCTGGGAAT |
| 3001 | TTCACTGCCA CGATAGAGGA GTGGCAAGCA GAGCATGATG CCATCATGAG GCACATCTTG |
| 3061 | GAGAGACCGG ACCCTACCGA CGTCTTCCAG AATAAGGCAA ACGTGTGTTG GGCCAAGGCT |
| 3121 | TTAGTGCCGG TGCTGAAGAC CGCTGGCATA GACATGACCA CTGAACAATG GAACACTGTG |
| 3181 | GATTATTTTG AAACGGACAA AGCTCACTCA GCAGAGATAG TATTGAACCA ACTATGCGTG |
| 3241 | AGGTTCTTTG GACTCGATCT GGACTCCGGT CTATTTTCTG CACCCACTGT TCCGTTATCC |
| 3301 | ATTAGGAATA ATCACTGGGA TAACTCCCCG TCGCCTAACA TGTACGGGCT GAATAAAGAA |
| 3361 | GTGGTCCGTC AGCTCTCTCG CAGGTACCCA CAACTGCCTC GGGCAGTTGC CACTGGAAGA |
| 3421 | GTCTATGACA TGAACACTGG TACACTGCGC AATTATGATC CGCGCATAAA CCTAGTACCT |
| 3481 | GTAAACAGAA GACTGCCTCA TGCTTTAGTC CTCCACCATA ATGAACACCC ACAGAGTGAC |
| 3541 | TTTTCTTCAT TCGTCAGCAA ATTGAAGGGC AGAACTGTCC TGGTGGTCGG GGAAAAGTTG |
| 3601 | TCCGTCCCAG GCAAAATGGT TGACTGGTTG TCAGACCGGC CTGAGGCTAC CTTCAGAGCT |
| 3661 | CGGCTGGATT TAGGCATCCC AGGTGATGTG CCCAAATATG ACATAATATT TGTTAATGTG |
| 3721 | AGGACCCCAT ATAAATACCA TCACTATCAG CAGTGTGAAG ACCATGCCAT TAAGCTTAGC |
| 3781 | ATGTTGACCA AGAAAGCTTG TCTGCATCTG AATCCCGGCG GAACCTGTGT CAGCATAGGT |
| 3841 | TATGGTTACG CTGACAGGGC CAGCGAAAGC ATCATTGGTG CTATAGCGCG GCAGTTCAAG |
| 3901 | TTTTCCCGGG TATGCAAACC GAAATCCTCA CTTGAAGAGA CGGAAGTTCT GTTTGTATTC |
| 3961 | ATTGGGTACG ATCGCAAGGC CCGTACGCAC AATCCTTACA AGCTTTCATC AACCTTGACC |
| 4021 | AACATTTATA CAGGTTCCAG ACTCCACGAA GCCGGATGTG CACCCTCATA TCATGTGGTG |
| 4081 | CGAGGGGATA TTGCCACGGC CACCGAAGGA GTGATTATAA ATGCTGCTAA CAGCAAAGGA |
| 4141 | CAACCTGGCG GAGGGGTGTG CGGAGCGCTG TATAAGAAAT TCCCGGAAAG CTTCGATTTA |
| 4201 | CAGCCGATCG AAGTAGGAAA AGCGCGACTG GTCAAAGGTG CAGCTAAACA TATCATTCAT |
| 4261 | GCCGTAGGAC CAAACTTCAA CAAAGTTTCG GAGGTTGAAG GTGACAAACA GTTGGCAGAG |
| 4321 | GCTTATGAGT CCATCGCTAA GATTGTCAAC GATAACAATT ACAAGTCAGT AGCGATTCCA |
| 4381 | CTGTTGTCCA CCGGCATCTT TTCCGGGAAC AAAGATCGAC TAACCCAATC ATTGAACCAT |
| 4441 | TTGCTGACAG CTTTAGACAC CACTGATGCA GATGTAGCCA TATACTGCAG GGACAAGAAA |
| 4501 | TGGGAAATGA CTCTCAAGGA AGCAGTGGCT AGGAGAGAAG CAGTGGAGGA GATATGCATA |
| 4561 | TCCGACGACT CTTCAGTGAC AGAACCTGAT GCAGAGCTGG TGAGGGTGCA TCCGAAGAGT |
| 4621 | TCTTTGGCTG GAAGGAAGGG CTACAGCACA AGCGATGGCA AAACTTTCTC ATATTTGGAA |
| 4681 | GGGACCAAGT TTCACCAGGC GGCCAAGGAT ATAGCAGAAA TTAATGCCAT GTGGCCCGTT |
| 4741 | GCAACGGAGG CCAATGAGCA GGTATGCATG TATATCCTCG GAGAAAGCAT GAGCAGTATT |
| 4801 | AGGTCGAAAT GCCCCGTCGA AGAGTCGGAA GCCTCCACAC CACCTAGCAC GCTGCCTTGC |
| 4861 | TTGTGCATCC ATGCCATGAC TCCAGAAAGA GTACAGCGCC TAAAAGCCTC ACGTCCAGAA |
| 4921 | CAAATTACTG TGTGCTCATC CTTTCCATTG CCGAAGTATA GAATCACTGG TGTGCAGAAG |
| 4981 | ATCCAATGCT CCCAGCCTAT ATTGTTCTCA CCGAAAGTGC CTGCGTATAT TCATCCAAGG |
| 5041 | AAGTATCTCG TGGAAACACC ACCGGTAGAC GAGACTCCGG AGCCATCGGC AGAGAACCAA |
| 5101 | TCCACAGAGG GGACACCTGA ACAACCACCA CTTATAACCG AGGATGAGAC CAGGACTAGA |
| 5161 | ACGCCTGAGC CGATCATCAT CGAAGAGGAA GAAGAGGATA GCATAAGTTT GCTGTCAGAT |
| 5221 | GGCCCGACCC ACCAGGTGCT GCAAGTCGAG GCAGACATTC ACGGGCCGCC CTCTGTATCT |
| 5281 | AGCTCATCCT GGTCCATTCC TCATGCATCC GACTTTGATG TGGACAGTTT ATCCATACTT |
| 5341 | GACACCCTGG AGGGAGCTAG CGTGACCAGC GGGGCAACGT CAGCCGAGAC TAACTCTTAC |
| 5401 | TTCGCAAAGA GTATGGAGTT TCTGGCGCGA CCGGTGCCTG CGCCTCGAAC AGTATTCAGG |
| 5461 | AACCCTCCAC ATCCCGCTCC GCGCACAAGA ACACCGTCAC TTGCACCCAG CAGGGCCTGC |
| 5521 | TCGAGAACCA GCCTAGTTTC CACCCCGCCA GGCGTGAATA GGGTGATCAC TAGAGAGGAG |
| 5581 | CTCGAGGCGC TTACCCCGTC ACGCACTCCT AGCAGGTCGG TCTCGAGAAC CAGCCTGGTC |
| 5641 | TCCAACCCGC CAGGCGTAAA TAGGGTGATT ACAAGAGAGG AGTTTGAGGC GTTCGTAGCA |
| 5701 | CAACAACAAT GACGGTTTGA TGCGGGTGCA TACATCTTTT CCTCCGACAC CGGTCAAGGG |
| 5761 | CATTTACAAC AAAAATCAGT AAGGCAAACG GTGCTATCCG AAGTGGTGTT GGAGAGGACC |
| 5821 | GAATTGGAGA TTTCGTATGC CCCGCGCCTC GACCAAGAAA AAGAAGAATT ACTACGCAAG |
| 5881 | AAATTACAGT TAAATCCCAC ACCTGCTAAC AGAAGCAGAT ACCAGTCCAG GAAGGTGGAG |
| 5941 | AACATGAAAG CCATAACAGC TAGACGTATT CTGCAAGGCC TAGGGCATTA TTTGAAGGCA |
| 6001 | GAAGGAAAAG TGGAGTGCTA CCGAACCCTG CATCCTGTTC CTTTGTATTC ATCTAGTGTG |
| 6061 | AACCGTGCCT TTTCAAGCCC CAAGGTCGCA GTGGAAGCCT GTAACGCCAT GTTGAAAGAG |
| 6121 | AACTTTCCGA CTGTGGCTTC TTACTGTATT ATTCCAGAGT ACGATGCCTA TTTGGACATG |
| 6181 | GTTGACGGAG CTTCATGCTG CTTAGACACT GCCAGTTTTT GCCCTGCAAA GCTGCGCAGC |
| 6241 | TTTCCAAAGA AACACTCCTA TTTGGAACCC ACAATACGAT CGGCAGTGCC TTCAGCGATC |
| 6301 | CAGAACACGC TCCAGAACGT CCTGGCAGCT GCCACAAAAA GAAATTGCAA TGTCACGCAA |
| 6361 | ATGAGAGAAT TGCCCGTATT GGATTCGGCG GCCTTTAATG TGGAATGCTT CAAGAAATAT |
| 6421 | GCGTGTAATA ATGAATATTG GGAAACGTTT AAAGAAAACC CCATCAGGCT TACTGAAGAA |
| 6481 | AACGTGGTAA ATTACATTAC CAAATTAAAA GGACCAAAAG CTGCTGCTCT TTTTGCGAAG |
| 6541 | ACACATAATT TGAATATGTT GCAGGACATA CCAATGGACA GGTTTGTAAT GGACTTAAAG |
| 6601 | AGAGACGTGA AAGTGACTCC AGGAACAAAA CATACTGAAG AACGGCCCAA GGTACAGGTG |
| 6661 | ATCCAGGCTG CCGATCCGCT AGCAACAGCG TATCTGTGCG GAATCCACCG AGAGCTGGTT |
| 6721 | AGGAGATTAA ATGCGGTCCT GCTTCCGAAC ATTCATACAC TGTTTGATAT GTCGGCTGAA |
| 6781 | GACTTTGACG CTATTATAGC CGAGCACTTC CAGCCTGGGG ATTGTGTTCT GGAAACTGAC |
| 6841 | ATCGCGTCGT TTGATAAAAG TGAGGACGAC GCCATGGCTC TGACCGCGTT AATGATTCTG |
| 6901 | GAAGACTTAG GTGTGGACGC AGAGCTGTTG ACGCTGATTG AGGCGGCTTT CGGCGAAATT |
| 6961 | TCATCAATAC ATTTGCCCAC TAAAACTAAA TTTAAATTCG GAGCCATGAT GAAATCTGGA |
| 7021 | ATGTTCCTCA CACTGTTTGT GAACACAGTC ATTAACATTG TAATCGCAAG CAGAGTGTTG |
| 7081 | AGAGAACGGC TAACCGGATC ACCATGTGCA GCATTCATTG GAGATGACAA TATCGTGAAA |
| 7141 | GGAGTCAAAT CGGACAAATT AATGGCAGAC AGGTGCGCCA CCTGGTTGAA TATGGAAGTC |
| 7201 | AAGATTATAG ATGCTGTGGT GGGCGAGAAA GCGCCTTATT TCTGTGGAGG GTTTATTTTG |
| 7261 | TGTGACTCCG TGACCGGCAC AGCGTGCCGT GTGGCAGACC CCCTAAAAAG GCTGTTTAAG |
| 7321 | CTTGGCAAAC CTCTGGCAGC AGACGATGAA CATGATGATG ACAGGAGAAG GGCATTGCAT |
| 7381 | GAAGAGTCAA CACGCTGGAA CCGAGTGGGT ATTCTTTCAG AGCTGTGCAA GGCAGTAGAA |
| 7441 | TCAAGGTATG AAACCGTAGG AACTTCCATC ATAGTTATGG CCATGACTAC TCTAGCTAGC |
| 7501 | AGTGTTAAAT CATTCAGCTA CCTGAGAGGG GCCCCTATAA CTCTCTACGG CTAACCTGAA |
| 7561 | TGGACTACGA CATAGTCTAG TCCGCCAAGG CCACCatggt gagcaagggc gaggagctgt |
| 7621 | tcaccggggt ggtgcccatc ctggtcgagc tggacggcga cgtaaacggc cacaagttca |
| 7681 | gcgtgtccgg cgagggcgag ggcgatgcca cctacggcaa gctgaccctg aagttcatct |
| 7741 | gcaccaccgg caagctgccc gtgccctggc ccaccctcgt gaccaccctg acctacggcg |
| 7801 | tgcagtgctt cagccgctac cccgaccaca tgaagcagca cgacttcttc aagtccgcca |
| 7861 | tgcccgaagg ctacgtccag gagcgcacca tcttcttcaa ggacgacggc aactacaaga |
| 7921 | cccgcgccga ggtgaagttc gagggcgaca ccctggtgaa ccgcatcgag ctgaagggca |
| 7981 | tcgacttcaa ggaggacggc aacatcctgg ggcacaagct ggagtacaac tacaacagcc |
| 8041 | acaacgtcta tatcatggcc gacaagcaga agaacggcat caaggtgaac ttcaagatcc |
| 8101 | gccacaacat cgaggacggc agcgtgcagc tcgccgacca ctaccagcag aacaccccca |
| 8161 | tcggcgacgg ccccgtgctg ctgcccgaca accactacct gagcacccag tccgccctga |
| 8221 | gcaaagaccc caacgagaag cgcgatcaca tggtcctgct ggagttcgtg accgccgccg |
| 8281 | ggatcactct cggcatggac gagctgtaca agtaaTGATA ATATGTTACG TGCAAAGGTG |
| 8341 | ATTGTCACCC CCCGAAAGAC CATATTGTGA CACACCCTCA GTATCACGCC CAAACATTTA |
| 8401 | CAGCCGCGGT GTCAAAAACC GCGTGGACGT GGTTAACATC CCTGCTGGGA GGATCAGCCG |
| 8461 | TAATTATTAT AATTGGCTTG GTGCTGGCTA CTATTGTGGC CATGTACGTG CTGACCAACC |
| 8521 | AGAAACATAA TTGAATACAG CAGCAATTGG CAAGCTGCTT ACATAGAACT CGCGGCGATT |
| 8581 | GGCATGCCGC CTTAAAATTT TTATTTTATT TTTCTTTTCT TTTCCGAATC GGATTTTGTT |
| 8641 | TTTAATATTT CAAAAAAAAA AAAAAAAAAA AAAAAAAAAA AAAAAAAAAA AAaaagaaga |
| 8701 | gcGCAGCTCT GGCCCGTGTC TCAAAATCTC TGATGTTACA TTGCACAAGA TAAAAATATA |
| 8761 | TCATCATGAA CAATAAAACT GTCTGCTTAC ATAAACAGTA ATACAAGGGG TGTTATGAGC |
| 8821 | CATATTCAAC GGGAAACGTC GAGGCCGCGA TTAAATTCCA ACATGGATGC TGATTTATAT |
| 8881 | GGGTATAAAT GGGCTCGCGA TAATGTCGGG CAATCAGGTG CGACAATCTA TCGCTTGTAT |
| 8941 | GGGAAGCCCG ATGCGCCAGA GTTGTTTCTG AAACATGGCA AAGGTAGCGT TGCCAATGAT |
| 9001 | GTTACAGATG AGATGGTCAG ACTAAACTGG CTGACGGAAT TTATGCCTCT TCCGACCATC |
| 9061 | AAGCATTTTA TCCGTACTCC TGATGATGCA TGGTTACTCA CCACTGCGAT CCCCGGAAAA |
| 9121 | ACAGCATTCC AGGTATTAGA AGAATATCCT GATTCAGGTG AAAATATTGT TGATGCGCTG |
| 9181 | GCAGTGTTCC TGCGCCGGTT GCATTCGATT CCTGTTTGTA ATTGTCCTTT TAACAGCGAT |
| 9241 | CGCGTATTTC GTCTCGCTCA GGCGCAATCA CGAATGAATA ACGGTTTGGT TGATGCGAGT |
| 9301 | GATTTTGATG ACGAGCGTAA TGGCTGGCCT GTTGAACAAG TCTGGAAAGA AATGCATAAA |
| 9361 | CTTTTGCCAT TCTCACCGGA TTCAGTCGTC ACTCATGGTG ATTTCTCACT TGATAACCTT |
| 9421 | ATTTTTGACG AGGGGAAATT AATAGGTTGT ATTGATGTTG GACGAGTCGG AATCGCAGAC |
| 9481 | CGATACCAGG ATCTTGCCAT CCTATGGAAC TGCCTCGGTG AGTTTTCTCC TTCATTACAG |
| 9541 | AAACGGCTTT TTCAAAAATA TGGTATTGAT AATCCTGATA TGAATAAATT GCAGTTTCAT |
| 9601 | TTGATGCTCG ATGAGTTTTT CTAATCAGAA TTGGTTAATT GGTTGTAACA CTGGCAGAGC |
| 9661 | ATTACGCTGA CTTGACGGGA CGGCGCAAGC TCATGACCAA AATCCCTTAA CGTGAGTTAC |
| 9721 | GCGTCGTTCC ACTGAGCGTC AGACCCCGTA GAAAAGATCA AAGGATCTTC TTGAGATCCT |
| 9781 | TTTTTTCTGC GCGTAATCTG CTGCTTGCAA ACAAAAAAAC CACCGCTACC AGCGGTGGTT |
| 9841 | TGTTTGCCGG ATCAAGAGCT ACCAACTCTT TTTCCGAAGG TAACTGGCTT CAGCAGAGCG |
| 9901 | CAGATACCAA ATACTGTTCT TCTAGTGTAG CCGTAGTTAG GCCACCACTT CAAGAACTCT |
| 9961 | GTAGCACCGC CTACATACCT CGCTCTGCTA ATCCTGTTAC CAGTGGCTGC TGCCAGTGGC |
| 10021 | GATAAGTCGT GTCTTACCGG GTTGGACTCA AGACGATAGT TACCGGATAA GGCGCAGCGG |
| 10081 | TCGGGCTGAA CGGGGGGTTC GTGCACACAG CCCAGCTTGG AGCGAACGAC CTACACCGAA |
| 10141 | CTGAGATACC TACAGCGTGA GCTATGAGAA AGCGCCACGC TTCCCGAAGG GAGAAAGGCG |
| 10201 | GACAGGTATC CGGTAAGCGG CAGGGTCGGA ACAGGAGAGC GCACGAGGGA GCTTCCAGGG |
| 10261 | GGAAACGCCT GGTATCTTTA TAGTCCTGTC GGGTTTCGCC ACCTCTGACT TGAGCGTCGA |
| 10321 | TTTTTGTGAT GCTCGTCAGG GGGGCGGAGC CTATGGAAAA ACGCCAGCAA CGCGGCCTTT |
| 10381 | TTACGGTTCC TGGCCTTTTG CTGGCCTTTT GCTCACAT |

Strand-mCherry (SEQ ID NO: 4)

| | |
|---|---|
| 1 | CGATACGGGA GGGCTTACCA TCTGGCCCCA GTGCTGCAAT GATACCGCGA GACCCACGCT |
| 61 | CACCGGCTCC AGATTTATCA GCAATAAACC AGCCAGCCGG AAGGGCCGAG CGCAGAAGTG |
| 121 | GTCCTGCAAC TTTATCCGCC TCCATCCAGT CTATTAATTG TTGCCGGGAA GCTAGAGTAA |
| 181 | GTAGTTCGCC AGTTAATAGT TTGCGCAACG TTGTTGCCAT TGCTACAGGC ATCGTGGTGT |
| 241 | CACGCTCGTC GTTTGGTATG GCTTCATTCA GCTCCGGTTC CCAACGATCA AGGCGAGTTA |
| 301 | CATGATCCCC CATGTTGTGC AAAAAAGCGG TTAGCTCCTT CGGTCCTCCG ATCGTTGTCA |
| 361 | GAAGTAAGTT GGCCGCAGTG TTATCACTCA TGGTTATGGC AGCACTGCAT AATTCTCTTA |
| 421 | CTGTCATGCC ATCCGTAAGA TGCTTTTCTG TGACTGGTGA GTACTCAACC AAGTCATTCT |
| 481 | GAGAATAGTG TATGCGGCGA CCGAGTTGCT CTTGCCCGGC GTCAATACGG GATAATACCG |
| 541 | CGCCACATAG CAGAACTTTA AAAGTGCTCA TCATTGGAAA ACGTTCTTCG GGGCGAAAAC |
| 601 | TCTCAAGGAT CTTACCGCTG TTGAGATCCA GTTCGATGTA ACCCACTCGT GCACCCAACT |
| 661 | GATCTTCAGC ATCTTTTACT TTCACCAGCG TTTCTGGGTG AGCAAAAACA GGAAGGCAAA |
| 721 | ATGCCGCAAA AAAGGGAATA AGGGCGACAC GGAAATGTTG AATACTCATA CTCTTCCTTT |
| 781 | TTCAATATTA TTGAAGCATT TATCAGGGTT ATTGTCTCAT GAGCGGATAC ATATTTGAAT |
| 841 | GTATTTAGAA AAATAAACAA ATAGGGGTTC CGCGCACATT TCCCCGAAAA GTGCCACCTG |
| 901 | ACGTTAGGGA TAACAGGGTA ATTAATACGA CTCACTATAA TGGGGGGCGC ATGAGAGAAG |
| 961 | CCCAGACCAA TTACCTACCC AAAATGGAGA AAGTTCACGT TGACATCGAG GAAGACAGCC |
| 1021 | CATTCCTCAG AGCTTTGCAG CGGAGCTTCC CGCAGTTTGA GGTAGAAGCC AAGCAGGTCA |
| 1081 | CTGATAATGA CCATGCTAAT GCCAGAGCGT TTTCGCATCT GGCTTCAAAA CTGATCGAAA |
| 1141 | CGGAGGTGGA CCCATCCGAC ACGATCCTTG ACATTGGAAG TGCGCCCGCC CGCAGAATGT |
| 1201 | ATTCTAAGCA CAAGTATCAT TGTATCTGTC CGATGAGATG TGCGGAAGAT CCGGACAGAT |
| 1261 | TGTATAAGTA TGCAACTAAG CTGAAGAAAA ACTGTAAGGA AATAACTGAT AAGGAATTGG |
| 1321 | ACAAGAAAAT GAAGGAGCTC GCCGCCGTCA TGAGCGACCC TGACCTGGAA ACTGAGACTA |
| 1381 | TGTGCCTCCA CGACGACGAG TCGTGTCGCT ACGAAGGGCA AGTCGCTGTT TACCAGGATG |
| 1441 | TATACGCGGT TGACGGACCG ACAAGTCTCT ATCACCAAGC CAATAAGGGA GTTAGAGTCG |
| 1501 | CCTACTGGAT AGGCTTTGAC ACCACCCCTT TTATGTTTAA GAACTTGGCT GGAGCATATC |
| 1561 | CATCATACTC TACCAACTGG GCCGACGAAA CCGTGTTAAC GGCTCGTAAC ATAGGCCTAT |
| 1621 | GCAGCTCTGA CGTTATGGAG CGGTCACGTA GAGGGATGTC CATTCTTAGA AAGAAGTATT |
| 1681 | TGAAACCATC CAACAATGTT CTATTCTCTG TTGGCTCGAC CATCTACCAC GAGAAGAGGG |
| 1741 | ACTTACTGAG GAGCTGGCAC CTGCCGTCTG TATTTCACTT ACGTGGCAAG CAAAATTACA |
| 1801 | CATGTCGGTG TGAGACTATA GTTAGTTGCG ACGGGTACGT CGTTAAAAGA ATAGCTATCA |
| 1861 | GTCCAGGCCT GTATGGGAAG CCTTCAGGCT ATGCTGCTAC GATGCACCGC GAGGGATTCT |
| 1921 | TGTGCTGCAA AGTGACAGAC ACATTGAACG GGGAGAGGGT CTCTTTTCCC GTGTGCACGT |
| 1981 | ATGTGCCAGC TACATTGTGT GACCAAATGA CTGGCATACT GGCAACAGAT GTCAGTGCGG |
| 2041 | ACGACGCGCA AAAACTGCTG GTTGGGCTCA ACCAGCGTAT AGTCGTCAAC GGTCGCACCC |
| 2101 | AGAGAAACAC CAATACCATG AAAAATTACC TTTTGCCCGT AGTGGCCCAG GCATTTGCTA |
| 2161 | GGTGGGCAAA GGAATATAAG GAAGATCAAG AAGATGAAAG GCCACTAGGA CTACGAGATA |
| 2221 | GACAGTTAGT CATGGGGTGT TGTTGGGCTT TTAGAAGGCA CAAGATAACA TCTATTTATA |
| 2281 | AGCGCCCGGA TACCCAAACC ATCATCAAAG TGAACAGCGA TTTCCACTCA TTCGTGCTGC |
| 2341 | CCAGGATAGG CAGTAACACA TTGGAGATCG GGCTGAGAAC AAGAATCAGG AAAATGTTAG |
| 2401 | AGGAGCACAA GGAGCCGTCA CCTCTCATTA CCGCCGAGGA CGTACAAGAA GCTAAGTGCG |
| 2461 | CAGCCGATGA GGCTAAGGAG GTGCGTGAAG CCGAGGAGTT GCGCGCAGCT CTACCACCTT |
| 2521 | TGGCAGCTGA TGTTGAGGAG CCCACTCTGG AAGCCGATGT CGACTTGATG TTACAAGAGG |
| 2581 | CTGGGGCCGG CTCAGTGGAG ACACCTCGTG GCTTGATAAA GGTTACCAGC TACGATGGCG |
| 2641 | AGGACAAGAT CGGCTCTTAC GCTGTGCTTT CTCCGCAGGC TGTACTCAAG AGTGAAAAAT |
| 2701 | TATCTTGCAT CCACCCTCTC GCTGAACAAG TCATAGTGAT AACACACTCT GGCCGAAAAG |
| 2761 | GGCGTTATGC CGTGGAACCA TACCATGGTA AAGTAGTGGT GCCAGAGGGA CATGCAATAC |
| 2821 | CCGTCCAGGA CTTTCAAGCT CTGAGTGAAA GTGCCACCAT TGTGTACAAC GAACGTGAGT |
| 2881 | TCGTAAACAG GTACCTGCAC CATATTGCCA CACATGGAGG AGCGCTGAAC ACTGATGAAG |
| 2941 | AATATTACAA AACTGTCAAG CCCAGCGAGC ACGACGGCGA ATACCTGTAC GACATCGACA |
| 3001 | GGAAACAGTG CGTCAAGAAA GAACTAGTCA CTGGGCTAGG GCTCACAGGC GAGCTGGTGG |
| 3061 | ATCCTCCCTT CCATGAATTC GCCTACGAGA GTCTGAGAAC ACGACCAGCC GCTCCTTACC |
| 3121 | AAGTACCAAC CATAGGGGTG TATGGCGTGC CAGGATCAGG CAAGTCTGGC ATCATTAAAA |
| 3181 | GCGCAGTCAC CAAAAAAGAT CTAGTGGTGA GCGCCAAGAA AGAAAACTGT GCAGAAATTA |
| 3241 | TAAGGGACGT CAAGAAAATG AAAGGGCTGG ACGTCAATGC CAGAACTGTG GACTCAGTGC |
| 3301 | TCTTGAATGG ATGCAAACAC CCCGTAGAGA CCCTGTATAT TGACGAAGCT TTTGCTTGTC |
| 3361 | ATGCAGGTAC TCTCAGAGCG CTCATAGCCA TTATAAGACC TAAAAAGGCA GTGCTCTGCG |
| 3421 | GGGATCCCAA ACAGTGCGGT TTTTTTAACA TGATGTGCCT GAAAGTGCAT TTTAACCACG |
| 3481 | AGATTTGCAC ACAAGTCTTC CACAAAAGCA TCTCTCGCCG TTGCACTAAA TCTGTGACTT |
| 3541 | CGGTCGTCTC AACCTTGTTT TACGACAAAA AAATGAGAAC GACGAATCCG AAAGAGACTA |
| 3601 | AGATTGTGAT TGACACTACC GGCAGTACCA AACCTAAGCA GGACGATCTC ATTCTCACTT |
| 3661 | GTTTCAGAGG GTGGGTGAAG CAGTTGCAAA TAGATTACAA AGGCAACGAA ATAATGACGG |
| 3721 | CAGCTGCCTC TCAAGGGCTG ACCCGTAAAG GTGTGTATGC CGTTCGGTAC AAGGTGAATG |
| 3781 | AAAATCCTCT GTACGCACCC ACCTCAGAAC ATGTGAACGT CCTACTGACC CGCACGGAGG |
| 3841 | ACCGCATCGT GTGGAAAACA CTAGCCGGCG ACCCATGGAT AAAAACACTG ACTGCCAAGT |
| 3901 | ACCCTGGGAA TTTCACTGCC ACGATAGAGG AGTGGCAAGC AGAGCATGAT GCCATCATGA |
| 3961 | GGCACATCTT GGAGAGACCG GACCCTACCG ACGTCTTCCA GAATAAGGCA AACGTGTGTT |
| 4021 | GGGCCAAGGC TTTAGTGCCG GTGCTGAAGA CCGCTGGCAT AGACATGACC ACTGAACAAT |
| 4081 | GGAACACTGT GGATTATTTT GAAACGGACA AAGCTCACTC AGCAGAGATA GTATTGAACC |
| 4141 | AACTATGCGT GAGGTTCTTT GGACTCGATC TGGACTCCGG TCTATTTTCT GCACCCACTG |
| 4201 | TTCCGTTATC CATTAGGAAT AATCACTGGG ATAACTCCCC GTCGCCTAAC ATGTACGGGC |
| 4261 | TGAATAAAGA AGTGGTCCGT CAGCTCTCTC GCAGGTACCC ACAACTGCCT CGGGCAGTTG |
| 4321 | CCACTGGAAG AGTCTATGAC ATGAACACTG GTACACTGCG CAATTATGAT CCGCGCATAA |
| 4381 | ACCTAGTACC TGTAAACAGA AGACTGCCTC ATGCTTTAGT CCTCCACCAT AATGAACACC |
| 4441 | CACAGAGTGA CTTTTCTTCA TTCGTCAGCA AATTGAAGGG CAGAACTGTC CTGGTGGTCG |
| 4501 | GGGAAAAGTT GTCCGTCCCA GGCAAAATGG TTGACTGGTT GTCAGACCGG CCTGAGGCTA |
| 4561 | CCTTCAGAGC TCGGCTGGAT TTAGGCATCC CAGGTGATGT GCCCAAATAT GACATAATAT |
| 4621 | TTGTTAATGT GAGGACCCCA TATAAATACC ATCACTATCA GCAGTGTGAA GACCATGCCA |
| 4681 | TTAAGCTTAG CATGTTGACC AAGAAAGCTT GTCTGCATCT GAATCCCGGC GGAACCTGTG |
| 4741 | TCAGCATAGG TTATGGTTAC GCTGACAGGG CCAGCGAAAG CATCATTGGT GCTATAGCGC |
| 4801 | GGCAGTTCAA GTTTTCCCGG GTATGCAAAC CGAAATCCTC ACTTGAAGAG ACGGAAGTTC |
| 4861 | TGTTTGTATT CATTGGGTAC GATCGCAAGG CCCGTACGCA CAATCCTTAC AAGCTTTCAT |
| 4921 | CAACCTTGAC CAACATTTAT ACAGGTTCCA GACTCCACGA AGCCGGATGT GCACCCTCAT |
| 4981 | ATCATGTGGT GCGAGGGGAT ATTGCCACGG CCACCGAAGG AGTGATTATA AATGCTGCTA |
| 5041 | ACAGCAAAGG ACAACCTGGC GGAGGGGTGT GCGGAGCGCT GTATAAGAAA TTCCCGGAAA |
| 5101 | GCTTCGATTT ACAGCCGATC GAAGTAGGAA AAGCGCGACT GGTCAAAGGT GCAGCTAAAC |
| 5161 | ATATCATTCA TGCCGTAGGA CCAAACTTCA ACAAAGTTTC GGAGGTTGAA GGTGACAAAC |
| 5221 | AGTTGGCAGA GGCTTATGAG TCCATCGCTA AGATTGTCAA CGATAACAAT TACAAGTCAG |
| 5281 | TAGCGATTCC ACTGTTGTCC ACCGGCATCT TTTCCGGGAA CAAAGATCGA CTAACCCAAT |
| 5341 | CATTGAACCA TTTGCTGACA GCTTTAGACA CCACTGATGC AGATGTAGCC ATATACTGCA |
| 5401 | GGGACAAGAA ATGGGAAATG ACTCTCAAGG AAGCAGTGGC TAGGAGAGAA GCAGTGGAGG |
| 5461 | AGATATGCAT ATCCGACGAC TCTTCAGTGA CAGAACCTGA TGCAGAGCTG GTGAGGGTGC |
| 5521 | ATCCGAAGAG TTCTTTGGCT GGAAGGAAGG GCTACAGCAC AAGCGATGGC AAAACTTTCT |
| 5581 | CATATTTGGA AGGGACCAAG TTTCACCAGG CGGCCAAGGA TATAGCAGAA ATTAATGCCA |
| 5641 | TGTGGCCCGT TGCAACGGAG GCCAATGAGC AGGTATGCAT GTATATCCTC GGAGAAAGCA |
| 5701 | TGAGCAGTAT TAGGTCGAAA TGCCCCGTCG AAGAGTCGGA AGCCTCCACA CCACCTAGCA |
| 5761 | CGCTGCCTTG CTTGTGCATC CATGCCATGA CTCCAGAAAG AGTACAGCGC CTAAAAGCCT |
| 5821 | CACGTCCAGA ACAAATTACT GTGTGCTCAT CCTTTCCATT GCCGAAGTAT AGAATCACTG |
| 5881 | GTGTGCAGAA GATCCAATGC TCCCAGCCTA TATTGTTCTC ACCGAAAGTG CCTGCGTATA |
| 5941 | TTCATCCAAG GAAGTATCTC GTGGAAACAC CACCGGTAGA CGAGACTCCG GAGCCATCGG |
| 6001 | CAGAGAACCA ATCCACAGAG GGGACACCTG AACAACCACC ACTTATAACC GAGGATGAGA |
| 6061 | CCAGGACTAG AACGCCTGAG CCGATCATCA TCGAAGAGGA AGAAGAGGAT AGCATAAGTT |
| 6121 | TGCTGTCAGA TGGCCCGACC CACCAGGTGC TGCAAGTCGA GGCAGACATT CACGGGCCGC |
| 6181 | CCTCTGTATC TAGCTCATCC TGGTCCATTC CTCATGCATC CGACTTTGAT GTGGACAGTT |
| 6241 | TATCCATACT TGACACCCTG GAGGGAGCTA GCGTGACCAG CGGGGCAACG TCAGCCGAGA |
| 6301 | CTAACTCTTA CTTCGCAAAG AGTATGGAGT TTCTGGCGCG ACCGGTGCCT GCGCCTCGAA |
| 6361 | CAGTATTCAG GAACCCTCCA CATCCCGCTC CGCGCACAAG AACACCGTCA CTTGCACCCA |
| 6421 | GCAGGGCCTG CTCGAGAACC AGCCTAGTTT CCACCCCGCC AGGCGTGAAT AGGGTGATCA |
| 6481 | CTAGAGAGGA GCTCGAGGCG CTTACCCCGT CACGCACTCC TAGCAGGTCG GTCTCGAGAA |
| 6541 | CCAGCCTGGT CTCCAACCCG CCAGGCGTAA ATAGGGTGAT TACAAGAGAG GAGTTTGAGG |
| 6601 | CGTTCGTAGC ACAACAACAA TGACGGTTTG ATGCGGGTGC ATACATCTTT TCCTCCGACA |
| 6661 | CCGGTCAAGG GCATTTACAA CAAAAATCAG TAAGGCAAAC GGTGCTATCC GAAGTGGTGT |
| 6721 | TGGAGAGGAC CGAATTGGAG ATTTCGTATG CCCCGCGCCT CGACCAAGAA AAAGAAGAAT |
| 6781 | TACTACGCAA GAAATTACAG TTAAATCCCA CACCTGCTAA CAGAAGCAGA TACCAGTCCA |
| 6841 | GGAAGGTGGA GAACATGAAA GCCATAACAG CTAGACGTAT TCTGCAAGGC CTAGGGCATT |
| 6901 | ATTTGAAGGC AGAAGGAAAA GTGGAGTGCT ACCGAACCCT GCATCCTGTT CCTTTGTATT |
| 6961 | CATCTAGTGT GAACCGTGCC TTTTCAAGCC CCAAGGTCGC AGTGGAAGCC TGTAACGCCA |
| 7021 | TGTTGAAAGA GAACTTTCCG ACTGTGGCTT CTTACTGTAT TATTCCAGAG TACGATGCCT |
| 7081 | ATTTGGACAT GGTTGACGGA GCTTCATGCT GCTTAGACAC TGCCAGTTTT TGCCCTGCAA |
| 7141 | AGCTGCGCAG CTTTCCAAAG AAACACTCCT ATTTGGAACC CACAATACGA TCGGCAGTGC |
| 7201 | CTTCAGCGAT CCAGAACACG CTCCAGAACG TCCTGGCAGC TGCCACAAAA AGAAATTGCA |
| 7261 | ATGTCACGCA AATGAGAGAA TTGCCCGTAT TGGATTCGGC GGCCTTTAAT GTGGAATGCT |
| 7321 | TCAAGAAATA TGCGTGTAAT AATGAATATT GGGAAACGTT TAAAGAAAAC CCCATCAGGC |
| 7381 | TTACTGAAGA AAACGTGGTA AATTACATTA CCAAATTAAA AGGACCAAAA GCTGCTGCTC |
| 7441 | TTTTTGCGAA GACACATAAT TTGAATATGT TGCAGGACAT ACCAATGGAC AGGTTTGTAA |
| 7501 | TGGACTTAAA GAGAGACGTG AAAGTGACTC CAGGAACAAA ACATACTGAA GAACGGCCCA |
| 7561 | AGGTACAGGT GATCCAGGCT GCCGATCCGC TAGCAACAGC GTATCTGTGC GGAATCCACC |
| 7621 | GAGAGCTGGT TAGGAGATTA AATGCGGTCC TGCTTCCGAA CATTCATACA CTGTTTGATA |
| 7681 | TGTCGGCTGA AGACTTTGAC GCTATTATAG CCGAGCACTT CCAGCCTGGG GATTGTGTTC |
| 7741 | TGGAAACTGA CATCGCGTCG TTTGATAAAA GTGAGGACGA CGCCATGGCT CTGACCGCGT |
| 7801 | TAATGATTCT GGAAGACTTA GGTGTGGACG CAGAGCTGTT GACGCTGATT GAGGCGGCTT |
| 7861 | TCGGCGAAAT TTCATCAATA CATTTGCCCA CTAAAACTAA ATTTAAATTC GGAGCCATGA |
| 7921 | TGAAATCTGG AATGTTCCTC ACACTGTTTG TGAACACAGT CATTAACATT GTAATCGCAA |
| 7981 | GCAGAGTGTT GAGAGAACGG CTAACCGGAT CACCATGTGC AGCATTCATT GGAGATGACA |
| 8041 | ATATCGTGAA AGGAGTCAAA TCGGACAAAT TAATGGCAGA CAGGTGCGCC ACCTGGTTGA |
| 8101 | ATATGGAAGT CAAGATTATA GATGCTGTGG TGGGCGAGAA AGCGCCTTAT TTCTGTGGAG |
| 8161 | GGTTTATTTT GTGTGACTCC GTGACCGGCA CAGCGTGCCG TGTGGCAGAC CCCCTAAAAA |
| 8221 | GGCTGTTTAA GCTTGGCAAA CCTCTGGCAG CAGACGATGA ACATGATGAT GACAGGAGAA |
| 8281 | GGGCATTGCA TGAAGAGTCA ACACGCTGGA ACCGAGTGGG TATTCTTTCA GAGCTGTGCA |
| 8341 | AGGCAGTAGA ATCAAGGTAT GAAACCGTAG GAACTTCCAT CATAGTTATG GCCATGACTA |
| 8401 | CTCTAGCTAG CAGTGTTAAA TCATTCAGCT ACCTGAGAGG GGCCCCTATA ACTCTCTACG |
| 8461 | GCTAACCTGA ATGGACTACG ACATAGTCTA GTCCGCCAAG GCCACCATGG TGAGCAAGGG |
| 8521 | CGAGGAGGAT AACATGGCCA TCATCAAGGA GTTCATGCGC TTCAAGGTGC ACATGGAGGG |
| 8581 | CTCCGTGAAC GGCCACGAGT TCGAGATCGA GGGCGAGGGC GAGGGCCGCC CCTACGAGGG |
| 8641 | CACCCAGACC GCCAAGCTGA AGGTGACCAA GGGTGGECCC CTGCCCTTCG CCTGGGACAT |
| 8701 | CCTGTCCCCT CAGTTCATGT ACGGCTCCAA GGCCTACGTG AAGCACCCCG CCGACATCCC |
| 8761 | CGACTACTTG AAGCTGTCCT TCCCCGAGGG CTTCAAGTGG GAGCGCGTGA TGAACTTCGA |
| 8821 | GGACGGCGGC GTGGTGACCG TGACCCAGGA CTCCTCCCTG CAGGACGGCG AGTTCATCTA |
| 8881 | CAAGGTGAAG CTGCGCGGCA CCAACTTCCC CTCCGACGGC CCCGTAATGC AGAAGAAGAC |
| 8941 | CATGGGCTGG GAGGCCTCCT CCGAGCGGAT GTACCCCGAG GACGGCGCCC TGAAGGGCGA |
| 9001 | GATCAAGCAG AGGCTGAAGC TGAAGGACGG CGGCCACTAC GACGCTGAGG TCAAGACCAC |
| 9061 | CTACAAGGCC AAGAAGCCCG TGCAGCTGCC CGGCGCCTAC AACGTCAACA TCAAGTTGGA |
| 9121 | CATCACCTCC CACAACGAGG ACTACACCAT CGTGGAACAG TACGAACGCG CCGAGGGCCG |
| 9181 | CCACTCCACC GGCGGCATGG ACGAGCTGTA CAAGTAGATA ATATGTTACG TGCAAAGGTG |
| 9241 | ATTGTCACCC CCCGAAAGAC CATATTGTGA CACACCCTCA GTATCACGCC CAAACATTTA |
| 9301 | CAGCCGCGGT GTCAAAAACC GCGTGGACGT GGTTAACATC CCTGCTGGGA GGATCAGCCG |
| 9361 | TAATTATTAT AATTGGCTTG GTGCTGGCTA CTATTGTGGC CATGTACGTG CTGACCAACC |
| 9421 | AGAAACATAA TTGAATACAG CAGCAATTGG CAAGCTGCTT ACATAGAACT CGCGGCGATT |
| 9481 | GGCATGCCGC CTTAAAATTT TTATTTTATT TTTCTTTTCT TTTCCGAATC GGATTTTGTT |
| 9541 | TTTAATATTT CAAAAAAAAA AAAAAAAAAA AAAAAAAAAA AAAAAAAAAA ATAGGGATAA |
| 9601 | CAGGGTAATT GAGCAAAAGG CCAGCAAAAG GCCAGGAACC GTAAAAAGGC CGCGTTGCTG |
| 9661 | GCGTTTTTCC ATAGGCTCCG CCCCCCTGAC GAGCATCACA AAAATCGACG CTCAAGTCAG |
| 9721 | AGGTGGCGAA ACCCGACAGG ACTATAAAGA TACCAGGCGT TTCCCCCTGG AAGCTCCCTC |
| 9781 | GTGCGCTCTC CTGTTCCGAC CCTGCCGCTT ACCGGATACC TGTCCGCCTT TCTCCCTTCG |
| 9841 | GGAAGCGTGG CGCTTTCTCA TAGCTCACGC TGTAGGTATC TCAGTTCGGT GTAGGTCGTT |
| 9901 | CGCTCCAAGC TGGGCTGTGT GCACGAACCC CCCGTTCAGC CCGACCGCTG CGCCTTATCC |
| 9961 | GGTAACTATC GTCTTGAGTC CAACCCGGTA AGACACGACT TATCGCCACT GGCAGCAGCC |
| 10021 | ACTGGTAACA GGATTAGCAG AGCGAGGTAT GTAGGCGGTG CTACAGAGTT CTTGAAGTGG |
| 10081 | TGGCCTAACT ACGGCTACAC TAGAAGAACA GTATTTGGTA TCTGCGCTCT GCTGAAGCCA |
| 10141 | GTTACCTTCG GAAAAAGAGT TGGTAGCTCT TGATCCGGCA AACAAACCAC CGCTGGTAGC |
| 10201 | GGTGGTTTTT TTGTTTGCAA GCAGCAGATT ACGCGCAGAA AAAAAGGATC TCAAGAAGAT |
| 10261 | CCTTTGATCT TTTCTACGGG GTCTGACGCT CAGTGGAACG AAAACTCACG TTAAGGGATT |
| 10321 | TTGGTCATGA GATTATCAAA AAGGATCTTC ACCTAGATCC TTTTAAATTA AAAATGAAGT |
| 10381 | TTTAAATCAA TCTAAAGTAT ATATGAGTAA ACTTGGTCTG ACAGTTACCA ATGCTTAATC |
| 10441 | AGTGAGGCAC CTATCTCAGC GATCTGTCTA TTTCGTTCAT CCATAGTTGC CTGACTCCCC |
| 10501 | GTCGTGTAGA TAACTA |

Non-cytopathic-EGFP (SEQ ID NO: 5)

| | |
|---|---|
| 1 | gctcttctaa gTAATACGAC TCACTATAAT GGGCGGCGCA TGAGAGAAGC CCAGACCAAT |
| 61 | TACCTACCCA AAATGGAGAA AGTTCACGTT GACATCGAGG AAGACAGCCC ATTCCTCAGA |
| 121 | GCTTTGCAGC GGAGCTTCCC GCAGTTTGAG GTAGAAGCCA AGCAGGTCAC TGATAATGAC |
| 181 | CATGCTAATG CCAGAGCGTT TTCGCATCTG GCTTCAAAAC TGATCGAAAC GGAGGTGGAC |
| 241 | CCATCCGACA CGATCCTTGA CATTGGAAGT GCGCCCGCCC GCAGAATGTA TTCTAAGCAC |
| 301 | AAGTATCATT GTATCTGTCC GATGAGATGT GCGGAAGATC CGGACAGATT GTATAAGTAT |
| 361 | GCAACTAAGC TGAAGAAAAA CTGTAAGGAA ATAACTGATA AGGAATTGGA CAAGAAAATG |
| 421 | AAGGAGCTCG CCGCCGTCAT GAGCGACCCT GACCTGGAAA CTGAGACTAT GTGCCTCCAC |
| 481 | GACGACGAGT CGTGTCGCTA CGAAGGGCAA GTCGCTGTTT ACCAGGATGT ATACGCGGTT |
| 541 | GACGGACCGA CAAGTCTCTA TCACCAAGCC AATAAGGGAG TTAGAGTCGC CTACTGGATA |
| 601 | GGCTTTGACA CCACCCCTTT TATGTTTAAG AACTTGGCTG GAGCATATCC ATCATACTCT |
| 661 | ACCAACTGGG CCGACGAAAC CGTGTTAACG GCTCGTAACA TAGGCCTATG CAGCTCTGAC |
| 721 | GTTATGGAGC GGTCACGTAG AGGGATGTCC ATTCTTAGAA AGAAGTATTT GAAACCATCC |
| 781 | AACAATGTTC TATTCTCTGT TGGCTCGACC ATCTACCACG AGAAGAGGGA CTTACTGAGG |
| 841 | AGCTGGCACC TGCCGTCTGT ATTTCACTTA CGTGGCAAGC AAAATTACAC ATGTCGGTGT |
| 901 | GAGACTATAG TTAGTTGCGA CGGGTACGTC GTTAAAAGAA TAGCTATCAG TCCAGGCCTG |
| 961 | TATGGGAAGC CTTCAGGCTA TGCTGCTACG ATGCACCGCG AGGGATTCTT GTGCTGCAAA |
| 1021 | GTGACAGACA CATTGAACGG GGAGAGGGTC TCTTTTCCCG TGTGCACGTA TGTGCCAGCT |
| 1081 | ACATTGTGTG ACCAAATGAC TGGCATACTG GCAACAGATG TCAGTGCGGA CGACGCGCAA |
| 1141 | AAACTGCTGG TTGGGCTCAA CCAGCGTATA GTCGTCAACG GTCGCACCCA GAGAAACACC |
| 1201 | AATACCATGA AAAATTACCT TTTGCCCGTA GTGGCCCAGG CATTTGCTAG GTGGGCAAAG |
| 1261 | GAATATAAGG AAGATCAAGA AGATGAAAGG CCACTAGGAC TACGAGATAG ACAGTTAGTC |
| 1321 | ATGGGGTGTT GTTGGGCTTT TAGAAGGCAC AAGATAACAT CTATTTATAA GCGCCCGGAT |
| 1381 | ACCCAAACCA TCATCAAAGT GAACAGCGAT TTCCACTCAT TCGTGCTGCC CAGGATAGGC |
| 1441 | AGTAACACAT TGGAGATCGG GCTGAGAACA AGAATCAGGA AAATGTTAGA GGAGCACAAG |
| 1501 | GAGCCGTCAC CTCTCATTAC CGCCGAGGAC GTACAAGAAG CTAAGTGCGC AGCCGATGAG |
| 1561 | GCTAAGGAGG TGCGTGAAGC CGAGGAGTTG CGCGCAGCTC TACCACCTTT GGCAGCTGAT |
| 1621 | GTTGAGGAGC CCACTCTGGA AGCCGATGTC GACTTGATGT TACAAGAGGC TGGGGCCGGC |
| 1681 | TCAGTGGAGA CACCTCGTGG CTTGATAAAG GTTACCAGCT ACGATGGCGA GGACAAGATC |
| 1741 | GGCTCTTACG CTGTGCTTTC TCCGCAGGCT GTACTCAAGA GTGAAAAATT ATCTTGCATC |
| 1801 | CACCCTCTCG CTGAACAAGT CATAGTGATA ACACACTCTG GCCGAAAAGG GCGTTATGCC |
| 1861 | GTGGAACCAT ACCATGGTAA AGTAGTGGTG CCAGAGGGAC ATGCAATACC CGTCCAGGAC |
| 1921 | TTTCAAGCTC TGAGTGAAAG TGCCACCATT GTGTACAACG AACGTGAGTT CGTAAACAGG |
| 1981 | TACCTGCACC ATATTGCCAC ACATGGAGGA GCGCTGAACA CTGATGAAGA ATATTACAAA |
| 2041 | ACTGTCAAGC CCAGCGAGCA CGACGGCGAA TACCTGTACG ACATCGACAG GAAACAGTGC |
| 2101 | GTCAAGAAAG AACTAGTCAC TGGGCTAGGG CTCACAGGCG AGCTGGTGGA TCCTCCCTTC |
| 2161 | CATGAATTCG CCTACGAGAG TCTGAGAACA CGACCAGCCG CTCCTTACCA AGTACCAACC |
| 2221 | ATAGGGGTGT ATGGCGTGCC AGGATCAGGC AAGTCTGGCA TCATTAAAAG CGCAGTCACC |
| 2281 | AAAAAAGATC TAGTGGTGAG CGCCAAGAAA GAAAACTGTG CAGAAATTAT AAGGGACGTC |
| 2341 | AAGAAAATGA AAGGGCTGGA CGTCAATGCC AGAACTGTGG ACTCAGTGCT CTTGAATGGA |
| 2401 | TGCAAACACC CCGTAGAGAC CCTGTATATT GACGAAGCTT TTGCTTGTCA TGCAGGTACT |
| 2461 | CTCAGAGCGC TCATAGCCAT TATAAGACCT AAAAAGGCAG TGCTCTGCGG GGATCCCAAA |
| 2521 | CAGTGCGGTT TTTTTAACAT GATGTGCCTG AAAGTGCATT TTAACCACGA GATTTGCACA |
| 2581 | CAAGTCTTCC ACAAAAGCAT CTCTCGCCGT TGCACTAAAT CTGTGACTTC GGTCGTCTCA |
| 2641 | ACCTTGTTTT ACGACAAAAA AATGAGAACG ACGAATCCGA AAGAGACTAA GATTGTGATT |
| 2701 | GACACTACCG GCAGTACCAA ACCTAAGCAG GACGATCTCA TTCTCACTTG TTTCAGAGGG |
| 2761 | TGGGTGAAGC AGTTGCAAAT AGATTACAAA GGCAACGAAA TAATGACGGC AGCTGCCTCT |
| 2821 | CAAGGGCTGA CCCGTAAAGG TGTGTATGCC GTTCGGTACA AGGTGAATGA AAATCCTCTG |
| 2881 | TACGCACCCA CCTCAGAACA TGTGAACGTC CTACTGACCC GCACGGAGGA CCGCATCGTG |
| 2941 | TGGAAAACAC TAGCCGGCGA CCCATGGATA AAAACACTGA CTGCCAAGTA CCCTGGGAAT |
| 3001 | TTCACTGCCA CGATAGAGGA GTGGCAAGCA GAGCATGATG CCATCATGAG GCACATCTTG |
| 3061 | GAGAGACCGG ACCCTACCGA CGTCTTCCAG AATAAGGCAA ACGTGTGTTG GGCCAAGGCT |
| 3121 | TTAGTGCCGG TGCTGAAGAC CGCTGGCATA GACATGACCA CTGAACAATG GAACACTGTG |
| 3181 | GATTATTTTG AAACGGACAA AGCTCACTCA GCAGAGATAG TATTGAACCA ACTATGCGTG |
| 3241 | AGGTTCTTTG GACTCGATCT GGACTCCGGT CTATTTTCTG CACCCACTGT TCCGTTATCC |
| 3301 | ATTAGGAATA ATCACTGGGA TAACTCCCCG TCGCCTAACA TGTACGGGCT GAATAAAGAA |
| 3361 | GTGGTCCGTC AGCTCTCTCG CAGGTACCCA CAACTGCCTC GGGCAGTTGC CACTGGAAGA |
| 3421 | GTCTATGACA TGAACACTGG TACACTGCGC AATTATGATC CGCGCATAAA CCTAGTACCT |
| 3481 | GTAAACAGAA GACTGCCTCA TGCTTTAGTC CTCCACCATA ATGAACACCC ACAGAGTGAC |
| 3541 | TTTTCTTCAT TCGTCAGCAA ATTGAAGGGC AGAACTGTCC TGGTGGTCGG GGAAAAGTTG |
| 3601 | TCCGTCCCAG GCAAAATGGT TGACTGGTTG TCAGACCGGC CTGAGGCTAC CTTCAGAGCT |
| 3661 | CGGCTGGATT TAGGCATCCC AGGTGATGTG CCCAAATATG ACATAATATT TGTTAATGTG |
| 3721 | AGGACCCCAT ATAAATACCA TCACTATCAG CAGTGTGAAG ACCATGCCAT TAAGCTTAGC |
| 3781 | ATGTTGACCA AGAAAGCTTG TCTGCATCTG AATCCCGGCG GAACCTGTGT CAGCATAGGT |
| 3841 | TATGGTTACG CTGACAGGGC CAGCGAAAGC ATCATTGGTG CTATAGCGCG GCTGTTCAAG |
| 3901 | TTTTCCCGGG TATGCAAACC GAAATCCTCA CTTGAAGAGA CGGAAGTTCT GTTTGTATTC |
| 3961 | ATTGGGTACG ATCGCAAGGC CCGTACGCAC AATCCTTACA AGCTTTCATC AACCTTGACC |
| 4021 | AACATTTATA CAGGTTCCAG ACTCCACGAA GCCGGATGTG CACCCTCATA TCATGTGGTG |
| 4081 | CGAGGGGATA TTGCCACGGC CACCGAAGGA GTGATTATAA ATGCTGCTAA CAGCAAAGGA |
| 4141 | CAACCTGGCG GAGGGGTGTG CGGAGCGCTG TATAAGAAAT TCCCGGAAAG CTTCGATTTA |
| 4201 | CAGCCGATCG AAGTAGGAAA AGCGCGACTG GTCAAAGGTG CAGCTAAACA TATCATTCAT |
| 4261 | GCCGTAGGAC CAAACTTCAA CAAAGTTTCG GAGGTTGAAG GTGACAAACA GTTGGCAGAG |
| 4321 | GCTTATGAGT CCATCGCTAA GATTGTCAAC GATAACAATT ACAAGTCAGT AGCGATTCCA |
| 4381 | CTGTTGTCCA CCGGCATCTT TTCCGGGAAC AAAGATCGAC TAACCCAATC ATTGAACCAT |
| 4441 | TTGCTGACAG CTTTAGACAC CACTGATGCA GATGTAGCCA TATACTGCAG GGACAAGAAA |
| 4501 | TGGGAAATGA CTCTCAAGGA AGCAGTGGCT AGGAGAGAAG CAGTGGAGGA GATATGCATA |
| 4561 | TCCGACGACT CTTCAGTGAC AGAACCTGAT GCAGAGCTGG TGAGGGTGCA TCCGAAGAGT |
| 4621 | TCTTTGGCTG GAAGGAAGGG CTACAGCACA AGCGATGGCA AAACTTTCTC ATATTTGGAA |
| 4681 | GGGACCAAGT TTCACCAGGC GGCCAAGGAT ATAGCAGAAA TTAATGCCAT GTGGCCCGTT |
| 4741 | GCAACGGAGG CCAATGAGCA GGTATGCATG TATATCCTCG GAGAAAGCAT GAGCAGTATT |
| 4801 | AGGTCGAAAT GCCCCGTCGA AGAGTCGGAA GCCTCCACAC CACCTAGCAC GCTGCCTTGC |
| 4861 | TTGTGCATCC ATGCCATGAC TCCAGAAAGA GTACAGCGCC TAAAAGCCTC ACGTCCAGAA |
| 4921 | CAAATTACTG TGTGCTCATC CTTTCCATTG CCGAAGTATA GAATCACTGG TGTGCAGAAG |
| 4981 | ATCCAATGCT CCCAGCCTAT ATTGTTCTCA CCGAAAGTGC CTGCGTATAT TCATCCAAGG |
| 5041 | AAGTATCTCG TGGAAACACC ACCGGTAGAC GAGACTCCGG AGCCATCGGC AGAGAACCAA |
| 5101 | TCCACAGAGG GGACACCTGA ACAACCACCA CTTATAACCG AGGATGAGAC CAGGACTAGA |
| 5161 | ACGCCTGAGC CGATCATCAT CGAAGAGGAA GAAGAGGATA GCATAAGTTT GCTGTCAGAT |
| 5221 | GGCCCGACCC ACCAGGTGCT GCAAGTCGAG GCAGACATTC ACGGGCCGCC CTCTGTATCT |
| 5281 | AGCTCATCCT GGTCCATTCC TCATGCATCC GACTTTGATG TGGACAGTTT ATCCATACTT |
| 5341 | GACACCCTGG AGGGAGCTAG CGTGACCAGC GGGGCAACGT CAGCCGAGAC TAACTCTTAC |
| 5401 | TTCGCAAAGA GTATGGAGTT TCTGGCGCGA CCGGTGCCTG CGCCTCGAAC AGTATTCAGG |
| 5461 | AACCCTCCAC ATCCCGCTCC GCGCACAAGA ACACCGTCAC TTGCACCCAG CAGGGCCTGC |
| 5521 | TCGAGAACCA GCCTAGTTTC CACCCCGCCA GGCGTGAATA GGGTGATCAC TAGAGAGGAG |
| 5581 | CTCGAGGCGC TTACCCCGTC ACGCACTCCT AGCAGGTCGG TCTCGAGAAC CAGCCTGGTC |
| 5641 | TCCAACCCGC CAGGCGTAAA TAGGGTGATT ACAAGAGAGG AGTTTGAGGC GTTCGTAGCA |
| 5701 | CAACAACAAT GACGGTTTGA TGCGGGTGCA TACATCTTTT CCTCCGACAC CGGTCAAGGG |
| 5761 | CATTTACAAC AAAAATCAGT AAGGCAAACG GTGCTATCCG AAGTGGTGTT GGAGAGGACC |
| 5821 | GAATTGGAGA TTTCGTATGC CCCGCGCCTC GACCAAGAAA AAGAAGAATT ACTACGCAAG |
| 5881 | AAATTACAGT TAAATCCCAC ACCTGCTAAC AGAAGCAGAT ACCAGTCCAG GAAGGTGGAG |
| 5941 | AACATGAAAG CCATAACAGC TAGACGTATT CTGCAAGGCC TAGGGCATTA TTTGAAGGCA |
| 6001 | GAAGGAAAAG TGGAGTGCTA CCGAACCCTG CATCCTGTTC CTTTGTATTC ATCTAGTGTG |
| 6061 | AACCGTGCCT TTTCAAGCCC CAAGGTCGCA GTGGAAGCCT GTAACGCCAT GTTGAAAGAG |
| 6121 | AACTTTCCGA CTGTGGCTTC TTACTGTATT ATTCCAGAGT ACGATGCCTA TTTGGACATG |
| 6181 | GTTGACGGAG CTTCATGCTG CTTAGACACT GCCAGTTTTT GCCCTGCAAA GCTGCGCAGC |
| 6241 | TTTCCAAAGA AACACTCCTA TTTGGAACCC ACAATACGAT CGGCAGTGCC TTCAGCGATC |
| 6301 | CAGAACACGC TCCAGAACGT CCTGGCAGCT GCCACAAAAA GAAATTGCAA TGTCACGCAA |
| 6361 | ATGAGAGAAT TGCCCGTATT GGATTCGGCG GCCTTTAATG TGGAATGCTT CAAGAAATAT |
| 6421 | GCGTGTAATA ATGAATATTG GGAAACGTTT AAAGAAAACC CCATCAGGCT TACTGAAGAA |
| 6481 | AACGTGGTAA ATTACATTAC CAAATTAAAA GGACCAAAAG CTGCTGCTCT TTTTGCGAAG |
| 6541 | ACACATAATT TGAATATGTT GCAGGACATA CCAATGGACA GGTTTGTAAT GGACTTAAAG |
| 6601 | AGAGACGTGA AAGTGACTCC AGGAACAAAA CATACTGAAG AACGGCCCAA GGTACAGGTG |
| 6661 | ATCCAGGCTG CCGATCCGCT AGCAACAGCG TATCTGTGCG GAATCCACCG AGAGCTGGTT |
| 6721 | AGGAGATTAA ATGCGGTCCT GCTTCCGAAC ATTCATACAC TGTTTGATAT GTCGGCTGAA |
| 6781 | GACTTTGACG CTATTATAGC CGAGCACTTC CAGCCTGGGG ATTGTGTTCT GGAAACTGAC |
| 6841 | ATCGCGTCGT TTGATAAAAG TGAGGACGAC GCCATGGCTC TGACCGCGTT AATGATTCTG |
| 6901 | GAAGACTTAG GTGTGGACGC AGAGCTGTTG ACGCTGATTG AGGCGGCTTT CGGCGAAATT |
| 6961 | TCATCAATAC ATTTGCCCAC TAAAACTAAA TTTAAATTCG GAGCCATGAT GAAATCTGGA |
| 7021 | ATGTTCCTCA CACTGTTTGT GAACACAGTC ATTAACATTG TAATCGCAAG CAGAGTGTTG |
| 7081 | AGAGAACGGC TAACCGGATC ACCATGTGCA GCATTCATTG GAGATGACAA TATCGTGAAA |
| 7141 | GGAGTCAAAT CGGACAAATT AATGGCAGAC AGGTGCGCCA CCTGGTTGAA TATGGAAGTC |
| 7201 | AAGATTATAG ATGCTGTGGT GGGCGAGAAA GCGCCTTATT TCTGTGGAGG GTTTATTTTG |
| 7261 | TGTGACTCCG TGACCGGCAC AGCGTGCCGT GTGGCAGACC CCCTAAAAAG GCTGTTTAAG |
| 7321 | CTTGGCAAAC CTCTGGCAGC AGACGATGAA CATGATGATG ACAGGAGAAG GGCATTGCAT |
| 7381 | GAAGAGTCAA CACGCTGGAA CCGAGTGGGT ATTCTTTCAG AGCTGTGCAA GGCAGTAGAA |
| 7441 | TCAAGGTATG AAACCGTAGG AACTTCCATC ATAGTTATGG CCATGACTAC TCTAGCTAGC |
| 7501 | AGTGTTAAAT CATTCAGCTA CCTGAGAGGG GCCCCTATAA CTCTCTACGG CTAACCTGAA |
| 7561 | TGGACTACGA CATAGTCTAG TCCGCCAAGG CCACCatggt gagcaagggc gaggagctgt |
| 7621 | tcaccggggt ggtgcccatc ctggtcgagc tggacggcga cgtaaacggc cacaagttca |
| 7681 | gcgtgtccgg cgagggcgag ggcgatgcca cctacggcaa gctgaccctg aagttcatct |
| 7741 | gcaccaccgg caagctgccc gtgccctggc ccaccctcgt gaccaccctg acctacggcg |
| 7801 | tgcagtgctt cagccgctac cccgaccaca tgaagcagca cgacttcttc aagtccgcca |
| 7861 | tgcccgaagg ctacgtccag gagcgcacca tottcttcaa ggacgacggc aactacaaga |
| 7921 | cccgcgccga ggtgaagttc gagggcgaca ccctggtgaa ccgcatcgag ctgaagggca |
| 7981 | tcgacttcaa ggaggacggc aacatcctgg ggcacaagct ggagtacaac tacaacagcc |
| 8041 | acaacgtcta tatcatggcc gacaagcaga agaacggcat caaggtgaac ttcaagatcc |
| 8101 | gccacaacat cgaggacggc agcgtgcagc tcgccgacca ctaccagcag aacaccccca |
| 8161 | tcggcgacgg ccccgtgctg ctgcccgaca accactacct gagcacccag tccgccctga |
| 8221 | gcaaagaccc caacgagaag cgcgatcaca tggtcctgct ggagttcgtg accgccgccg |
| 8281 | ggatcactct cggcatggac gagctgtaca agtaaTGATA ATATGTTACG TGCAAAGGTG |
| 8341 | ATTGTCACCC CCCGAAAGAC CATATTGTGA CACACCCTCA GTATCACGCC CAAACATTTA |
| 8401 | CAGCCGCGGT GTCAAAAACC GCGTGGACGT GGTTAACATC CCTGCTGGGA GGATCAGCCG |
| 8461 | TAATTATTAT AATTGGCTTG GTGCTGGCTA CTATTGTGGC CATGTACGTG CTGACCAACC |
| 8521 | AGAAACATAA TTGAATACAG CAGCAATTGG CAAGCTGCTT ACATAGAACT CGCGGCGATT |
| 8581 | GGCATGCCGC CTTAAAATTT TTATTTTATT TTTCTTTTCT TTTCCGAATC GGATTTTGTT |
| 8641 | TTTAATATTT CAAAAAAAAA AAAAAAAAAA AAAAAAAAAA AAAAAAAAAA AAaaagaaga |
| 8701 | gcGCAGCTCT GGCCCGTGTC TCAAAATCTC TGATGTTACA TTGCACAAGA TAAAAATATA |
| 8761 | TCATCATGAA CAATAAAACT GTCTGCTTAC ATAAACAGTA ATACAAGGGG TGTTATGAGC |
| 8821 | CATATTCAAC GGGAAACGTC GAGGCCGCGA TTAAATTCCA ACATGGATGC TGATTTATAT |
| 8881 | GGGTATAAAT GGGCTCGCGA TAATGTCGGG CAATCAGGTG CGACAATCTA TCGCTTGTAT |
| 8941 | GGGAAGCCCG ATGCGCCAGA GTTGTTTCTG AAACATGGCA AAGGTAGCGT TGCCAATGAT |
| 9001 | GTTACAGATG AGATGGTCAG ACTAAACTGG CTGACGGAAT TTATGCCTCT TCCGACCATC |
| 9061 | AAGCATTTTA TCCGTACTCC TGATGATGCA TGGTTACTCA CCACTGCGAT CCCCGGAAAA |
| 9121 | ACAGCATTCC AGGTATTAGA AGAATATCCT GATTCAGGTG AAAATATTGT TGATGCGCTG |
| 9181 | GCAGTGTTCC TGCGCCGGTT GCATTCGATT CCTGTTTGTA ATTGTCCTTT TAACAGCGAT |
| 9241 | CGCGTATTTC GTCTCGCTCA GGCGCAATCA CGAATGAATA ACGGTTTGGT TGATGCGAGT |
| 9301 | GATTTTGATG ACGAGCGTAA TGGCTGGCCT GTTGAACAAG TCTGGAAAGA AATGCATAAA |
| 9361 | CTTTTGCCAT TCTCACCGGA TTCAGTCGTC ACTCATGGTG ATTTCTCACT TGATAACCTT |
| 9421 | ATTTTTGACG AGGGGAAATT AATAGGTTGT ATTGATGTTG GACGAGTCGG AATCGCAGAC |
| 9481 | CGATACCAGG ATCTTGCCAT CCTATGGAAC TGCCTCGGTG AGTTTTCTCC TTCATTACAG |
| 9541 | AAACGGCTTT TTCAAAAATA TGGTATTGAT AATCCTGATA TGAATAAATT GCAGTTTCAT |
| 9601 | TTGATGCTCG ATGAGTTTTT CTAATCAGAA TTGGTTAATT GGTTGTAACA CTGGCAGAGC |
| 9661 | ATTACGCTGA CTTGACGGGA CGGCGCAAGC TCATGACCAA AATCCCTTAA CGTGAGTTAC |
| 9721 | GCGTCGTTCC ACTGAGCGTC AGACCCCGTA GAAAAGATCA AAGGATCTTC TTGAGATCCT |
| 9781 | TTTTTTCTGC GCGTAATCTG CTGCTTGCAA ACAAAAAAAC CACCGCTACC AGCGGTGGTT |
| 9841 | TGTTTGCCGG ATCAAGAGCT ACCAACTCTT TTTCCGAAGG TAACTGGCTT CAGCAGAGCG |
| 9901 | CAGATACCAA ATACTGTTCT TCTAGTGTAG CCGTAGTTAG GCCACCACTT CAAGAACTCT |
| 9961 | GTAGCACCGC CTACATACCT CGCTCTGCTA ATCCTGTTAC CAGTGGCTGC TGCCAGTGGC |
| 10021 | GATAAGTCGT GTCTTACCGG GTTGGACTCA AGACGATAGT TACCGGATAA GGCGCAGCGG |
| 10081 | TCGGGCTGAA CGGGGGGTTC GTGCACACAG CCCAGCTTGG AGCGAACGAC CTACACCGAA |
| 10141 | CTGAGATACC TACAGCGTGA GCTATGAGAA AGCGCCACGC TTCCCGAAGG GAGAAAGGCG |
| 10201 | GACAGGTATC CGGTAAGCGG CAGGGTCGGA ACAGGAGAGC GCACGAGGGA GCTTCCAGGG |
| 10261 | GGAAACGCCT GGTATCTTTA TAGTCCTGTC GGGTTTCGCC ACCTCTGACT TGAGCGTCGA |
| 10321 | TTTTTGTGAT GCTCGTCAGG GGGGCGGAGC CTATGGAAAA ACGCCAGCAA CGCGGCCTTT |
| 10381 | TTACGGTTCC TGGCCTTTTG CTGGCCTTTT GCTCACAT |

Strand-NS1-EGFP (SEQ ID NO: 6)

| | |
|---|---|
| 1 | gctcttctaa gTAATACGAC TCACTATAAT GGGCGGCGCA TGAGAGAAGC CCAGACCAAT |
| 61 | TACCTACCCA AAATGGAGAA AGTTCACGTT GACATCGAGG AAGACAGCCC ATTCCTCAGA |
| 121 | GCTTTGCAGC GGAGCTTCCC GCAGTTTGAG GTAGAAGCCA AGCAGGTCAC TGATAATGAC |
| 181 | CATGCTAATG CCAGAGCGTT TTCGCATCTG GCTTCAAAAC TGATCGAAAC GGAGGTGGAC |
| 241 | CCATCCGACA CGATCCTTGA CATTGGAAGT GCGCCCGCCC GCAGAATGTA TTCTAAGCAC |
| 301 | AAGTATCATT GTATCTGTCC GATGAGATGT GCGGAAGATC CGGACAGATT GTATAAGTAT |
| 361 | GCAACTAAGC TGAAGAAAAA CTGTAAGGAA ATAACTGATA AGGAATTGGA CAAGAAAATG |
| 421 | AAGGAGCTCG CCGCCGTCAT GAGCGACCCT GACCTGGAAA CTGAGACTAT GTGCCTCCAC |
| 481 | GACGACGAGT CGTGTCGCTA CGAAGGGCAA GTCGCTGTTT ACCAGGATGT ATACGCGGTT |
| 541 | GACGGACCGA CAAGTCTCTA TCACCAAGCC AATAAGGGAG TTAGAGTCGC CTACTGGATA |
| 601 | GGCTTTGACA CCACCCCTTT TATGTTTAAG AACTTGGCTG GAGCATATCC ATCATACTCT |
| 661 | ACCAACTGGG CCGACGAAAC CGTGTTAACG GCTCGTAACA TAGGCCTATG CAGCTCTGAC |
| 721 | GTTATGGAGC GGTCACGTAG AGGGATGTCC ATTCTTAGAA AGAAGTATTT GAAACCATCC |
| 781 | AACAATGTTC TATTCTCTGT TGGCTCGACC ATCTACCACG AGAAGAGGGA CTTACTGAGG |
| 841 | AGCTGGCACC TGCCGTCTGT ATTTCACTTA CGTGGCAAGC AAAATTACAC ATGTCGGTGT |
| 901 | GAGACTATAG TTAGTTGCGA CGGGTACGTC GTTAAAAGAA TAGCTATCAG TCCAGGCCTG |
| 961 | TATGGGAAGC CTTCAGGCTA TGCTGCTACG ATGCACCGCG AGGGATTCTT GTGCTGCAAA |
| 1021 | GTGACAGACA CATTGAACGG GGAGAGGGTC TCTTTTCCCG TGTGCACGTA TGTGCCAGCT |
| 1081 | ACATTGTGTG ACCAAATGAC TGGCATACTG GCAACAGATG TCAGTGCGGA CGACGCGCAA |
| 1141 | AAACTGCTGG TTGGGCTCAA CCAGCGTATA GTCGTCAACG GTCGCACCCA GAGAAACACC |
| 1201 | AATACCATGA AAAATTACCT TTTGCCCGTA GTGGCCCAGG CATTTGCTAG GTGGGCAAAG |
| 1261 | GAATATAAGG AAGATCAAGA AGATGAAAGG CCACTAGGAC TACGAGATAG ACAGTTAGTC |
| 1321 | ATGGGGTGTT GTTGGGCTTT TAGAAGGCAC AAGATAACAT CTATTTATAA GCGCCCGGAT |
| 1381 | ACCCAAACCA TCATCAAAGT GAACAGCGAT TTCCACTCAT TCGTGCTGCC CAGGATAGGC |
| 1441 | AGTAACACAT TGGAGATCGG GCTGAGAACA AGAATCAGGA AAATGTTAGA GGAGCACAAG |
| 1501 | GAGCCGTCAC CTCTCATTAC CGCCGAGGAC GTACAAGAAG CTAAGTGCGC AGCCGATGAG |
| 1561 | GCTAAGGAGG TGCGTGAAGC CGAGGAGTTG CGCGCAGCTC TACCACCTTT GGCAGCTGAT |
| 1621 | GTTGAGGAGC CCACTCTGGA AGCCGATGTC GACTTGATGT TACAAGAGGC TGGGGCCGGC |
| 1681 | TCAGTGGAGA CACCTCGTGG CTTGATAAAG GTTACCAGCT ACGATGGCGA GGACAAGATC |
| 1741 | GGCTCTTACG CTGTGCTTTC TCCGCAGGCT GTACTCAAGA GTGAAAAATT ATCTTGCATC |
| 1801 | CACCCTCTCG CTGAACAAGT CATAGTGATA ACACACTCTG GCCGAAAAGG GCGTTATGCC |
| 1861 | GTGGAACCAT ACCATGGTAA AGTAGTGGTG CCAGAGGGAC ATGCAATACC CGTCCAGGAC |
| 1921 | TTTCAAGCTC TGAGTGAAAG TGCCACCATT GTGTACAACG AACGTGAGTT CGTAAACAGG |
| 1981 | TACCTGCACC ATATTGCCAC ACATGGAGGA GCGCTGAACA CTGATGAAGA ATATTACAAA |
| 2041 | ACTGTCAAGC CCAGCGAGCA CGACGGCGAA TACCTGTACG ACATCGACAG GAAACAGTGC |
| 2101 | GTCAAGAAAG AACTAGTCAC TGGGCTAGGG CTCACAGGCG AGCTGGTGGA TCCTCCCTTC |
| 2161 | CATGAATTCG CCTACGAGAG TCTGAGAACA CGACCAGCCG CTCCTTACCA AGTACCAACC |
| 2221 | ATAGGGGTGT ATGGCGTGCC AGGATCAGGC AAGTCTGGCA TCATTAAAAG CGCAGTCACC |
| 2281 | AAAAAAGATC TAGTGGTGAG CGCCAAGAAA GAAAACTGTG CAGAAATTAT AAGGGACGTC |
| 2341 | AAGAAAATGA AAGGGCTGGA CGTCAATGCC AGAACTGTGG ACTCAGTGCT CTTGAATGGA |
| 2401 | TGCAAACACC CCGTAGAGAC CCTGTATATT GACGAAGCTT TTGCTTGTCA TGCAGGTACT |
| 2461 | CTCAGAGCGC TCATAGCCAT TATAAGACCT AAAAAGGCAG TGCTCTGCGG GGATCCCAAA |
| 2521 | CAGTGCGGTT TTTTTAACAT GATGTGCCTG AAAGTGCATT TTAACCACGA GATTTGCACA |
| 2581 | CAAGTCTTCC ACAAAAGCAT CTCTCGCCGT TGCACTAAAT CTGTGACTTC GGTCGTCTCA |
| 2641 | ACCTTGTTTT ACGACAAAAA AATGAGAACG ACGAATCCGA AAGAGACTAA GATTGTGATT |
| 2701 | GACACTACCG GCAGTACCAA ACCTAAGCAG GACGATCTCA TTCTCACTTG TTTCAGAGGG |
| 2761 | TGGGTGAAGC AGTTGCAAAT AGATTACAAA GGCAACGAAA TAATGACGGC AGCTGCCTCT |
| 2821 | CAAGGGCTGA CCCGTAAAGG TGTGTATGCC GTTCGGTACA AGGTGAATGA AAATCCTCTG |
| 2881 | TACGCACCCA CCTCAGAACA TGTGAACGTC CTACTGACCC GCACGGAGGA CCGCATCGTG |
| 2941 | TGGAAAACAC TAGCCGGCGA CCCATGGATA AAAACACTGA CTGCCAAGTA CCCTGGGAAT |
| 3001 | TTCACTGCCA CGATAGAGGA GTGGCAAGCA GAGCATGATG CCATCATGAG GCACATCTTG |
| 3061 | GAGAGACCGG ACCCTACCGA CGTCTTCCAG AATAAGGCAA ACGTGTGTTG GGCCAAGGCT |
| 3121 | TTAGTGCCGG TGCTGAAGAC CGCTGGCATA GACATGACCA CTGAACAATG GAACACTGTG |
| 3181 | GATTATTTTG AAACGGACAA AGCTCACTCA GCAGAGATAG TATTGAACCA ACTATGCGTG |
| 3241 | AGGTTCTTTG GACTCGATCT GGACTCCGGT CTATTTTCTG CACCCACTGT TCCGTTATCC |
| 3301 | ATTAGGAATA ATCACTGGGA TAACTCCCCG TCGCCTAACA TGTACGGGCT GAATAAAGAA |
| 3361 | GTGGTCCGTC AGCTCTCTCG CAGGTACCCA CAACTGCCTC GGGCAGTTGC CACTGGAAGA |
| 3421 | GTCTATGACA TGAACACTGG TACACTGCGC AATTATGATC CGCGCATAAA CCTAGTACCT |
| 3481 | GTAAACAGAA GACTGCCTCA TGCTTTAGTC CTCCACCATA ATGAACACCC ACAGAGTGAC |
| 3541 | TTTTCTTCAT TCGTCAGCAA ATTGAAGGGC AGAACTGTCC TGGTGGTCGG GGAAAAGTTG |
| 3601 | TCCGTCCCAG GCAAAATGGT TGACTGGTTG TCAGACCGGC CTGAGGCTAC CTTCAGAGCT |
| 3661 | CGGCTGGATT TAGGCATCCC AGGTGATGTG CCCAAATATG ACATAATATT TGTTAATGTG |
| 3721 | AGGACCCCAT ATAAATACCA TCACTATCAG CAGTGTGAAG ACCATGCCAT TAAGCTTAGC |
| 3781 | ATGTTGACCA AGAAAGCTTG TCTGCATCTG AATCCCGGCG GAACCTGTGT CAGCATAGGT |
| 3841 | TATGGTTACG CTGACAGGGC CAGCGAAAGC ATCATTGGTG CTATAGCGCG GCAGTTCAAG |
| 3901 | TTTTCCCGGG TATGCAAACC GAAATCCTCA CTTGAAGAGA CGGAAGTTCT GTTTGTATTC |
| 3961 | ATTGGGTACG ATCGCAAGGC CCGTACGCAC AATCCTTACA AGCTTTCATC AACCTTGACC |
| 4021 | AACATTTATA CAGGTTCCAG ACTCCACGAA GCCGGATGTG CACCCTCATA TCATGTGGTG |
| 4081 | CGAGGGGATA TTGCCACGGC CACCGAAGGA GTGATTATAA ATGCTGCTAA CAGCAAAGGA |
| 4141 | CAACCTGGCG GAGGGGTGTG CGGAGCGCTG TATAAGAAAT TCCCGGAAAG CTTCGATTTA |
| 4201 | CAGCCGATCG AAGTAGGAAA AGCGCGACTG GTCAAAGGTG CAGCTAAACA TATCATTCAT |
| 4261 | GCCGTAGGAC CAAACTTCAA CAAAGTTTCG GAGGTTGAAG GTGACAAACA GTTGGCAGAG |
| 4321 | GCTTATGAGT CCATCGCTAA GATTGTCAAC GATAACAATT ACAAGTCAGT AGCGATTCCA |
| 4381 | CTGTTGTCCA CCGGCATCTT TTCCGGGAAC AAAGATCGAC TAACCCAATC ATTGAACCAT |
| 4441 | TTGCTGACAG CTTTAGACAC CACTGATGCA GATGTAGCCA TATACTGCAG GGACAAGAAA |
| 4501 | TGGGAAATGA CTCTCAAGGA AGCAGTGGCT AGGAGAGAAG CAGTGGAGGA GATATGCATA |
| 4561 | TCCGACGACT CTTCAGTGAC AGAACCTGAT GCAGAGCTGG TGAGGGTGCA TCCGAAGAGT |
| 4621 | TCTTTGGCTG GAAGGAAGGG CTACAGCACA AGCGATGGCA AAACTTTCTC ATATTTGGAA |
| 4681 | GGGACCAAGT TTCACCAGGC GGCCAAGGAT ATAGCAGAAA TTAATGCCAT GTGGCCCGTT |
| 4741 | GCAACGGAGG CCAATGAGCA GGTATGCATG TATATCCTCG GAGAAAGCAT GAGCAGTATT |
| 4801 | AGGTCGAAAT GCCCCGTCGA AGAGTCGGAA GCCTCCACAC CACCTAGCAC GCTGCCTTGC |
| 4861 | TTGTGCATCC ATGCCATGAC TCCAGAAAGA GTACAGCGCC TAAAAGCCTC ACGTCCAGAA |
| 4921 | CAAATTACTG TGTGCTCATC CTTTCCATTG CCGAAGTATA GAATCACTGG TGTGCAGAAG |
| 4981 | ATCCAATGCT CCCAGCCTAT ATTGTTCTCA CCGAAAGTGC CTGCGTATAT TCATCCAAGG |
| 5041 | AAGTATCTCG TGGAAACACC ACCGGTAGAC GAGACTCCGG AGCCATCGGC AGAGAACCAA |
| 5101 | TCCACAGAGG GGACACCTGA ACAACCACCA CTTATAACCG AGGATGAGAC CAGGACTAGA |
| 5161 | ACGCCTGAGC CGATCATCAT CGAAGAGGAA GAAGAGGATA GCATAAGTTT GCTGTCAGAT |
| 5221 | GGCCCGACCC ACCAGGTGCT GCAAGTCGAG GCAGACATTC ACGGGCCGCC CTCTGTATCT |
| 5281 | AGCTCATCCT GGTCCATTCC TCATGCATCC GACTTTGATG TGGACAGTTT ATCCATACTT |
| 5341 | GACACCCTGG AGGGAGCTAG CGTGACCAGC GGGGCAACGT CAGCCGAGAC TAACTCTTAC |
| 5401 | TTCGCAAAGA GTATGGAGTT TCTGGCGCGA CCGGTGCCTG CGCCTCGAAC AGTATTCAGG |
| 5461 | AACCCTCCAC ATCCCGCTCC GCGCACAAGA ACACCGTCAC TTGCACCCAG CAGGGCCTGC |
| 5521 | TCGAGAACCA GCCTAGTTTC CACCCCGCCA GGCGTGAATA GGGTGATCAC TAGAGAGGAG |
| 5581 | CTCGAGGCGC TTACCCCGTC ACGCACTCCT AGCAGGTCGG TCTCGAGAAC CAGCCTGGTC |
| 5641 | TCCAACCCGC CAGGCGTAAA TAGGGTGATT ACAAGAGAGG AGTTTGAGGC GTTCGTAGCA |
| 5701 | CAACAACAAT GACGGTTTGA TGCGGGTGCA TACATCTTTT CCTCCGACAC CGGTCAAGGG |
| 5761 | CATTTACAAC AAAAATCAGT AAGGCAAACG GTGCTATCCG AAGTGGTGTT GGAGAGGACC |
| 5821 | GAATTGGAGA TTTCGTATGC CCCGCGCCTC GACCAAGAAA AAGAAGAATT ACTACGCAAG |
| 5881 | AAATTACAGT TAAATCCCAC ACCTGCTAAC AGAAGCAGAT ACCAGTCCAG GAAGGTGGAG |
| 5941 | AACATGAAAG CCATAACAGC TAGACGTATT CTGCAAGGCC TAGGGCATTA TTTGAAGGCA |
| 6001 | GAAGGAAAAG TGGAGTGCTA CCGAACCCTG CATCCTGTTC CTTTGTATTC ATCTAGTGTG |
| 6061 | AACCGTGCCT TTTCAAGCCC CAAGGTCGCA GTGGAAGCCT GTAACGCCAT GTTGAAAGAG |
| 6121 | AACTTTCCGA CTGTGGCTTC TTACTGTATT ATTCCAGAGT ACGATGCCTA TTTGGACATG |
| 6181 | GTTGACGGAG CTTCATGCTG CTTAGACACT GCCAGTTTTT GCCCTGCAAA GCTGCGCAGC |
| 6241 | TTTCCAAAGA AACACTCCTA TTTGGAACCC ACAATACGAT CGGCAGTGCC TTCAGCGATC |
| 6301 | CAGAACACGC TCCAGAACGT CCTGGCAGCT GCCACAAAAA GAAATTGCAA TGTCACGCAA |
| 6361 | ATGAGAGAAT TGCCCGTATT GGATTCGGCG GCCTTTAATG TGGAATGCTT CAAGAAATAT |
| 6421 | GCGTGTAATA ATGAATATTG GGAAACGTTT AAAGAAAACC CCATCAGGCT TACTGAAGAA |
| 6481 | AACGTGGTAA ATTACATTAC CAAATTAAAA GGACCAAAAG CTGCTGCTCT TTTTGCGAAG |
| 6541 | ACACATAATT TGAATATGTT GCAGGACATA CCAATGGACA GGTTTGTAAT GGACTTAAAG |
| 6601 | AGAGACGTGA AAGTGACTCC AGGAACAAAA CATACTGAAG AACGGCCCAA GGTACAGGTG |
| 6661 | ATCCAGGCTG CCGATCCGCT AGCAACAGCG TATCTGTGCG GAATCCACCG AGAGCTGGTT |
| 6721 | AGGAGATTAA ATGCGGTCCT GCTTCCGAAC ATTCATACAC TGTTTGATAT GTCGGCTGAA |
| 6781 | GACTTTGACG CTATTATAGC CGAGCACTTC CAGCCTGGGG ATTGTGTTCT GGAAACTGAC |
| 6841 | ATCGCGTCGT TTGATAAAAG TGAGGACGAC GCCATGGCTC TGACCGCGTT AATGATTCTG |
| 6901 | GAAGACTTAG GTGTGGACGC AGAGCTGTTG ACGCTGATTG AGGCGGCTTT CGGCGAAATT |
| 6961 | TCATCAATAC ATTTGCCCAC TAAAACTAAA TTTAAATTCG GAGCCATGAT GAAATCTGGA |
| 7021 | ATGTTCCTCA CACTGTTTGT GAACACAGTC ATTAACATTG TAATCGCAAG CAGAGTGTTG |
| 7081 | AGAGAACGGC TAACCGGATC ACCATGTGCA GCATTCATTG GAGATGACAA TATCGTGAAA |
| 7141 | GGAGTCAAAT CGGACAAATT AATGGCAGAC AGGTGCGCCA CCTGGTTGAA TATGGAAGTC |
| 7201 | AAGATTATAG ATGCTGTGGT GGGCGAGAAA GCGCCTTATT TCTGTGGAGG GTTTATTTTG |
| 7261 | TGTGACTCCG TGACCGGCAC AGCGTGCCGT GTGGCAGACC CCCTAAAAAG GCTGTTTAAG |
| 7321 | CTTGGCAAAC CTCTGGCAGC AGACGATGAA CATGATGATG ACAGGAGAAG GGCATTGCAT |
| 7381 | GAAGAGTCAA CACGCTGGAA CCGAGTGGGT ATTCTTTCAG AGCTGTGCAA GGCAGTAGAA |
| 7441 | TCAAGGTATG AAACCGTAGG AACTTCCATC ATAGTTATGG CCATGACTAC TCTAGCTAGC |
| 7501 | AGTGTTAAAT CATTCAGCTA CCTGAGAGGG GCCCCTATAA CTCTCTACGG CTAACCTGAA |
| 7561 | TGGACTACGA CATAGTCTAG TCCGCCAAGG CCACCatgga cagcaacacg gtgtcctcct |
| 7621 | tccaggtgga ctgcttcctc tggcacgtgc gcaagcgctt cgccgaccag gagctgggcg |
| 7681 | acgccccctt cctggaccgc cttcgccggg accagaagtc cctgcggggc cggggcagca |
| 7741 | cgcttggcct ggacatccgc acggccaccc gggaggggaa gcacatcgtg gagcggatcc |
| 7801 | tggaggagga gtcggacgag gccctgaaga tgacgatcgc gagcgtgccc gcgccccggt |
| 7861 | acctaaccga gatgacgctg gaggagatga gcagggactg gctgatgctc atccccaagc |
| 7921 | agaaggtgac cgggtccctc tgcatacgca tggaccaggc catcatggac aaggacatca |
| 7981 | tcctgaaggc caacttcagc gtcatcttta accggctgga ggccctcatc ctgctccgcg |
| 8041 | ccttcaccga cgagggggcc attgtggggg agatcagccc cctccccagc ctgccgggcc |
| 8101 | acaccgagga ggacgtcaag aacgccatcg gggtcctcat cggcggcctc gagtggaacg |
| 8161 | acaacaccgt ccgcgtgagc gagaccctcc agcggttcac gtggcgcagc tctgacgaga |
| 8221 | acggccggag ccccctcccg cccaagcaga agcggaagat ggagcggacg atcgagcccg |
| 8281 | aggtgGCTAC TAACTTCAGC CTGCTGAAGC AGGCTGGCGA CGTGGAGGAG AACCCTGGAC |
| 8341 | CTatggtgag caagggcgag gagctgttca ccggggtggt gcccatcctg gtcgagctgg |
| 8401 | acggcgacgt aaacggccac aagttcagcg tgtccggcga gggcgagggc gatgccacct |
| 8461 | acggcaagct gaccctgaag ttcatctgca ccaccggcaa gctgcccgtg ccctggccca |
| 8521 | ccctcgtgac caccctgacc tacggcgtgc agtgcttcag ccgctacccc gaccacatga |
| 8581 | agcagcacga cttcttcaag tccgccatgc ccgaaggcta cgtccaggag cgcaccatct |
| 8641 | tcttcaagga cgacggcaac tacaagaccc gcgccgaggt gaagttcgag ggcgacaccc |
| 8701 | tggtgaaccg catcgagctg aagggcatcg acttcaagga ggacggcaac atcctggggc |
| 8761 | acaagctgga gtacaactac aacagccaca acgtctatat catggccgac aagcagaaga |
| 8821 | acggcatcaa ggtgaacttc aagatccgcc acaacatcga ggacggcagc gtgcagctcg |
| 8881 | ccgaccacta ccagcagaac acccccatcg gcgacggccc cgtgctgctg cccgacaacc |
| 8941 | actacctgag cacccagtcc gccctgagca aagaccccaa cgagaagcgc gatcacatgg |
| 9001 | tcctgctgga gttcgtgacc gccgccggga tcactctcgg catggacgag ctgtacaagt |
| 9061 | aaTGATAATA TGTTACGTGC AAAGGTGATT GTCACCCCCC GAAAGACCAT ATTGTGACAC |
| 9121 | ACCCTCAGTA TCACGCCCAA ACATTTACAG CCGCGGTGTC AAAAACCGCG TGGACGTGGT |
| 9181 | TAACATCCCT GCTGGGAGGA TCAGCCGTAA TTATTATAAT TGGCTTGGTG CTGGCTACTA |
| 9241 | TTGTGGCCAT GTACGTGCTG ACCAACCAGA AACATAATTG AATACAGCAG CAATTGGCAA |
| 9301 | GCTGCTTACA TAGAACTCGC GGCGATTGGC ATGCCGCCTT AAAATTTTTA TTTTATTTTT |
| 9361 | CTTTTCTTTT CCGAATCGGA TTTTGTTTTT AATATTTCAA AAAAAAAAAA AAAAAAAAAA |
| 9421 | AAAAAAAAAA AAAAAAAAAa aagaagagcG CAGCTCTGGC CCGTGTCTCA AAATCTCTGA |
| 9481 | TGTTACATTG CACAAGATAA AAATATATCA TCATGAACAA TAAAACTGTC TGCTTACATA |
| 9541 | AACAGTAATA CAAGGGGTGT TATGAGCCAT ATTCAACGGG AAACGTCGAG GCCGCGATTA |
| 9601 | AATTCCAACA TGGATGCTGA TTTATATGGG TATAAATGGG CTCGCGATAA TGTCGGGCAA |
| 9661 | TCAGGTGCGA CAATCTATCG CTTGTATGGG AAGCCCGATG CGCCAGAGTT GTTTCTGAAA |
| 9721 | CATGGCAAAG GTAGCGTTGC CAATGATGTT ACAGATGAGA TGGTCAGACT AAACTGGCTG |
| 9781 | ACGGAATTTA TGCCTCTTCC GACCATCAAG CATTTTATCC GTACTCCTGA TGATGCATGG |
| 9841 | TTACTCACCA CTGCGATCCC CGGAAAAACA GCATTCCAGG TATTAGAAGA ATATCCTGAT |
| 9901 | TCAGGTGAAA ATATTGTTGA TGCGCTGGCA GTGTTCCTGC GCCGGTTGCA TTCGATTCCT |
| 9961 | GTTTGTAATT GTCCTTTTAA CAGCGATCGC GTATTTCGTC TCGCTCAGGC GCAATCACGA |
| 10021 | ATGAATAACG GTTTGGTTGA TGCGAGTGAT TTTGATGACG AGCGTAATGG CTGGCCTGTT |
| 10081 | GAACAAGTCT GGAAAGAAAT GCATAAACTT TTGCCATTCT CACCGGATTC AGTCGTCACT |
| 10141 | CATGGTGATT TCTCACTTGA TAACCTTATT TTTGACGAGG GGAAATTAAT AGGTTGTATT |
| 10201 | GATGTTGGAC GAGTCGGAAT CGCAGACCGA TACCAGGATC TTGCCATCCT ATGGAACTGC |
| 10261 | CTCGGTGAGT TTTCTCCTTC ATTACAGAAA CGGCTTTTTC AAAAATATGG TATTGATAAT |
| 10321 | CCTGATATGA ATAAATTGCA GTTTCATTTG ATGCTCGATG AGTTTTTCTA ATCAGAATTG |
| 10381 | GTTAATTGGT TGTAACACTG GCAGAGCATT ACGCTGACTT GACGGGACGG CGCAAGCTCA |
| 10441 | TGACCAAAAT CCCTTAACGT GAGTTACGCG TCGTTCCACT GAGCGTCAGA CCCCGTAGAA |
| 10501 | AAGATCAAAG GATCTTCTTG AGATCCTTTT TTTCTGCGCG TAATCTGCTG CTTGCAAACA |
| 10561 | AAAAAACCAC CGCTACCAGC GGTGGTTTGT TTGCCGGATC AAGAGCTACC AACTCTTTTT |
| 10621 | CCGAAGGTAA CTGGCTTCAG CAGAGCGCAG ATACCAAATA CTGTTCTTCT AGTGTAGCCG |
| 10681 | TAGTTAGGCC ACCACTTCAA GAACTCTGTA GCACCGCCTA CATACCTCGC TCTGCTAATC |
| 10741 | CTGTTACCAG TGGCTGCTGC CAGTGGCGAT AAGTCGTGTC TTACCGGGTT GGACTCAAGA |
| 10801 | CGATAGTTAC CGGATAAGGC GCAGCGGTCG GGCTGAACGG GGGGTTCGTG CACACAGCCC |
| 10861 | AGCTTGGAGC GAACGACCTA CACCGAACTG AGATACCTAC AGCGTGAGCT ATGAGAAAGC |
| 10921 | GCCACGCTTC CCGAAGGGAG AAAGGCGGAC AGGTATCCGG TAAGCGGCAG GGTCGGAACA |
| 10981 | GGAGAGCGCA CGAGGGAGCT TCCAGGGGGA AACGCCTGGT ATCTTTATAG TCCTGTCGGG |
| 11041 | TTTCGCCACC TCTGACTTGA GCGTCGATTT TTGTGATGCT CGTCAGGGGG GCGGAGCCTA |
| 11101 | TGGAAAAACG CCAGCAACGC GGCCTTTTTA CGGTTCCTGG CCTTTTGCTG GCCTTTTGCT |
| 11161 | CACAT |

Strand NS1-mCherry (SEQ ID NO: 7)

| | |
|---|---|
| 1 | ACAAAGGCAA CGAAATAATG ACGGCAGCTG CCTCTCAAGG GCTGACCCGT AAAGGTGTGT |
| 61 | ATGCCGTTCG GTACAAGGTG AATGAAAATC CTCTGTACGC ACCCACCTCA GAACATGTGA |
| 121 | ACGTCCTACT GACCCGCACG GAGGACCGCA TCGTGTGGAA AACACTAGCC GGCGACCCAT |
| 181 | GGATAAAAAC ACTGACTGCC AAGTACCCTG GGAATTTCAC TGCCACGATA GAGGAGTGGC |
| 241 | AAGCAGAGCA TGATGCCATC ATGAGGCACA TCTTGGAGAG ACCGGACCCT ACCGACGTCT |
| 301 | TCCAGAATAA GGCAAACGTG TGTTGGGCCA AGGCTTTAGT GCCGGTGCTG AAGACCGCTG |
| 361 | GCATAGACAT GACCACTGAA CAATGGAACA CTGTGGATTA TTTTGAAACG GACAAAGCTC |
| 421 | ACTCAGCAGA GATAGTATTG AACCAACTAT GCGTGAGGTT CTTTGGACTC GATCTGGACT |
| 481 | CCGGTCTATT TTCTGCACCC ACTGTTCCGT TATCCATTAG GAATAATCAC TGGGATAACT |
| 541 | CCCCGTCGCC TAACATGTAC GGGCTGAATA AAGAAGTGGT CCGTCAGCTC TCTCGCAGGT |
| 601 | ACCCACAACT GCCTCGGGCA GTTGCCACTG GAAGAGTCTA TGACATGAAC ACTGGTACAC |
| 661 | TGCGCAATTA TGATCCGCGC ATAAACCTAG TACCTGTAAA CAGAAGACTG CCTCATGCTT |
| 721 | TAGTCCTCCA CCATAATGAA CACCCACAGA GTGACTTTTC TTCATTCGTC AGCAAATTGA |
| 781 | AGGGCAGAAC TGTCCTGGTG GTCGGGGAAA AGTTGTCCGT CCCAGGCAAA ATGGTTGACT |
| 841 | GGTTGTCAGA CCGGCCTGAG GCTACCTTCA GAGCTCGGCT GGATTTAGGC ATCCCAGGTG |
| 901 | ATGTGCCCAA ATATGACATA ATATTTGTTA ATGTGAGGAC CCCATATAAA TACCATCACT |
| 961 | ATCAGCAGTG TGAAGACCAT GCCATTAAGC TTAGCATGTT GACCAAGAAA GCTTGTCTGC |
| 1021 | ATCTGAATCC CGGCGGAACC TGTGTCAGCA TAGGTTATGG TTACGCTGAC AGGGCCAGCG |
| 1081 | AAAGCATCAT TGGTGCTATA GCGCGGCAGT TCAAGTTTTC CCGGGTATGC AAACCGAAAT |
| 1141 | CCTCACTTGA AGAGACGGAA GTTCTGTTTG TATTCATTGG GTACGATCGC AAGGCCCGTA |
| 1201 | CGCACAATCC TTACAAGCTT TCATCAACCT TGACCAACAT TTATACAGGT TCCAGACTCC |
| 1261 | ACGAAGCCGG ATGTGCACCC TCATATCATG TGGTGCGAGG GGATATTGCC ACGGCCACCG |
| 1321 | AAGGAGTGAT TATAAATGCT GCTAACAGCA AAGGACAACC TGGCGGAGGG GTGTGCGGAG |
| 1381 | CGCTGTATAA GAAATTCCCG GAAAGCTTCG ATTTACAGCC GATCGAAGTA GGAAAAGCGC |
| 1441 | GACTGGTCAA AGGTGCAGCT AAACATATCA TTCATGCCGT AGGACCAAAC TTCAACAAAG |
| 1501 | TTTCGGAGGT TGAAGGTGAC AAACAGTTGG CAGAGGCTTA TGAGTCCATC GCTAAGATTG |
| 1561 | TCAACGATAA CAATTACAAG TCAGTAGCGA TTCCACTGTT GTCCACCGGC ATCTTTTCCG |
| 1621 | GGAACAAAGA TCGACTAACC CAATCATTGA ACCATTTGCT GACAGCTTTA GACACCACTG |
| 1681 | ATGCAGATGT AGCCATATAC TGCAGGGACA AGAAATGGGA AATGACTCTC AAGGAAGCAG |
| 1741 | TGGCTAGGAG AGAAGCAGTG GAGGAGATAT GCATATCCGA CGACTCTTCA GTGACAGAAC |
| 1801 | CTGATGCAGA GCTGGTGAGG GTGCATCCGA AGAGTTCTTT GGCTGGAAGG AAGGGCTACA |
| 1861 | GCACAAGCGA TGGCAAAACT TTCTCATATT TGGAAGGGAC CAAGTTTCAC CAGGCGGCCA |
| 1921 | AGGATATAGC AGAAATTAAT GCCATGTGGC CCGTTGCAAC GGAGGCCAAT GAGCAGGTAT |
| 1981 | GCATGTATAT CCTCGGAGAA AGCATGAGCA GTATTAGGTC GAAATGCCCC GTCGAAGAGT |
| 2041 | CGGAAGCCTC CACACCACCT AGCACGCTGC CTTGCTTGTG CATCCATGCC ATGACTCCAG |
| 2101 | AAAGAGTACA GCGCCTAAAA GCCTCACGTC CAGAACAAAT TACTGTGTGC TCATCCTTTC |
| 2161 | CATTGCCGAA GTATAGAATC ACTGGTGTGC AGAAGATCCA ATGCTCCCAG CCTATATTGT |
| 2221 | TCTCACCGAA AGTGCCTGCG TATATTCATC CAAGGAAGTA TCTCGTGGAA ACACCACCGG |
| 2281 | TAGACGAGAC TCCGGAGCCA TCGGCAGAGA ACCAATCCAC AGAGGGGACA CCTGAACAAC |
| 2341 | CACCACTTAT AACCGAGGAT GAGACCAGGA CTAGAACGCC TGAGCCGATC ATCATCGAAG |
| 2401 | AGGAAGAAGA GGATAGCATA AGTTTGCTGT CAGATGGCCC GACCCACCAG GTGCTGCAAG |
| 2461 | TCGAGGCAGA CATTCACGGG CCGCCCTCTG TATCTAGCTC ATCCTGGTCC ATTCCTCATG |
| 2521 | CATCCGACTT TGATGTGGAC AGTTTATCCA TACTTGACAC CCTGGAGGGA GCTAGCGTGA |
| 2581 | CCAGCGGGGC AACGTCAGCC GAGACTAACT CTTACTTCGC AAAGAGTATG GAGTTTCTGG |
| 2641 | CGCGACCGGT GCCTGCGCCT CGAACAGTAT TCAGGAACCC TCCACATCCC GCTCCGCGCA |
| 2701 | CAAGAACACC GTCACTTGCA CCCAGCAGGG CCTGCTCGAG AACCAGCCTA GTTTCCACCC |
| 2761 | CGCCAGGCGT GAATAGGGTG ATCACTAGAG AGGAGCTCGA GGCGCTTACC CCGTCACGCA |
| 2821 | CTCCTAGCAG GTCGGTCTCG AGAACCAGCC TGGTCTCCAA CCCGCCAGGC GTAAATAGGG |
| 2881 | TGATTACAAG AGAGGAGTTT GAGGCGTTCG TAGCACAACA ACAATGACGG TTTGATGCGG |
| 2941 | GTGCATACAT CTTTTCCTCC GACACCGGTC AAGGGCATTT ACAACAAAAA TCAGTAAGGC |
| 3001 | AAACGGTGCT ATCCGAAGTG GTGTTGGAGA GGACCGAATT GGAGATTTCG TATGCCCCGC |
| 3061 | GCCTCGACCA AGAAAAAGAA GAATTACTAC GCAAGAAATT ACAGTTAAAT CCCACACCTG |
| 3121 | CTAACAGAAG CAGATACCAG TCCAGGAAGG TGGAGAACAT GAAAGCCATA ACAGCTAGAC |
| 3181 | GTATTCTGCA AGGCCTAGGG CATTATTTGA AGGCAGAAGG AAAAGTGGAG TGCTACCGAA |
| 3241 | CCCTGCATCC TGTTCCTTTG TATTCATCTA GTGTGAACCG TGCCTTTTCA AGCCCCAAGG |
| 3301 | TCGCAGTGGA AGCCTGTAAC GCCATGTTGA AAGAGAACTT TCCGACTGTG GCTTCTTACT |
| 3361 | GTATTATTCC AGAGTACGAT GCCTATTTGG ACATGGTTGA CGGAGCTTCA TGCTGCTTAG |
| 3421 | ACACTGCCAG TTTTTGCCCT GCAAAGCTGC GCAGCTTTCC AAAGAAACAC TCCTATTTGG |
| 3481 | AACCCACAAT ACGATCGGCA GTGCCTTCAG CGATCCAGAA CACGCTCCAG AACGTCCTGG |
| 3541 | CAGCTGCCAC AAAAAGAAAT TGCAATGTCA CGCAAATGAG AGAATTGCCC GTATTGGATT |
| 3601 | CGGCGGCCTT TAATGTGGAA TGCTTCAAGA AATATGCGTG TAATAATGAA TATTGGGAAA |
| 3661 | CGTTTAAAGA AAACCCCATC AGGCTTACTG AAGAAAACGT GGTAAATTAC ATTACCAAAT |
| 3721 | TAAAAGGACC AAAAGCTGCT GCTCTTTTTG CGAAGACACA TAATTTGAAT ATGTTGCAGG |
| 3781 | ACATACCAAT GGACAGGTTT GTAATGGACT TAAAGAGAGA CGTGAAAGTG ACTCCAGGAA |
| 3841 | CAAAACATAC TGAAGAACGG CCCAAGGTAC AGGTGATCCA GGCTGCCGAT CCGCTAGCAA |
| 3901 | CAGCGTATCT GTGCGGAATC CACCGAGAGC TGGTTAGGAG ATTAAATGCG GTCCTGCTTC |
| 3961 | CGAACATTCA TACACTGTTT GATATGTCGG CTGAAGACTT TGACGCTATT ATAGCCGAGC |
| 4021 | ACTTCCAGCC TGGGGATTGT GTTCTGGAAA CTGACATCGC GTCGTTTGAT AAAAGTGAGG |
| 4081 | ACGACGCCAT GGCTCTGACC GCGTTAATGA TTCTGGAAGA CTTAGGTGTG GACGCAGAGC |
| 4141 | TGTTGACGCT GATTGAGGCG GCTTTCGGCG AAATTTCATC AATACATTTG CCCACTAAAA |
| 4201 | CTAAATTTAA ATTCGGAGCC ATGATGAAAT CTGGAATGTT CCTCACACTG TTTGTGAACA |
| 4261 | CAGTCATTAA CATTGTAATC GCAAGCAGAG TGTTGAGAGA ACGGCTAACC GGATCACCAT |
| 4321 | GTGCAGCATT CATTGGAGAT GACAATATCG TGAAAGGAGT CAAATCGGAC AAATTAATGG |
| 4381 | CAGACAGGTG CGCCACCTGG TTGAATATGG AAGTCAAGAT TATAGATGCT GTGGTGGGCG |
| 4441 | AGAAAGCGCC TTATTTCTGT GGAGGGTTTA TTTTGTGTGA CTCCGTGACC GGCACAGCGT |
| 4501 | GCCGTGTGGC AGACCCCCTA AAAAGGCTGT TTAAGCTTGG CAAACCTCTG GCAGCAGACG |
| 4561 | ATGAACATGA TGATGACAGG AGAAGGGCAT TGCATGAAGA GTCAACACGC TGGAACCGAG |
| 4621 | TGGGTATTCT TTCAGAGCTG TGCAAGGCAG TAGAATCAAG GTATGAAACC GTAGGAACTT |
| 4681 | CCATCATAGT TATGGCCATG ACTACTCTAG CTAGCAGTGT TAAATCATTC AGCTACCTGA |
| 4741 | GAGGGGCCCC TATAACTCTC TACGGCTAAC CTGAATGGAC TACGACATAG TCTAGTCCGC |
| 4801 | CAAGGCCACC atggacagca acacggtgtc ctccttccag gtggactgct tcctctggca |
| 4861 | cgtgcgcaag cgcttcgccg accaggagct gggcgacgcc cccttcctgg accgccttcg |
| 4921 | ccgggaccag aagtccctgc ggggccgggg cagcacgctt ggcctggaca tccgcacggc |
| 4981 | cacccgggag gggaagcaca tcgtggagcg gatcctggag gaggagtcgg acgaggccct |
| 5041 | gaagatgacg atcgcgagcg tgcccgcgcc ccggtaccta accgagatga cgctggagga |
| 5101 | gatgagcagg gactggctga tgctcatccc caagcagaag gtgaccgggt ccctctgcat |
| 5161 | acgcatggac caggccatca tggacaagga catcatcctg aaggccaact tcagcgtcat |
| 5221 | ctttaaccgg ctggaggccc tcatcctgct ccgcgccttc accgacgagg gggccattgt |
| 5281 | gggggagatc agccccctcc ccagcctgcc gggccacacc gaggaggacg tcaagaacgc |
| 5341 | catcggggtc ctcatcggcg gcctcgagtg gaacgacaac accgtccgcg tgagcgagac |
| 5401 | cctccagcgg ttcacgtggc gcagctctga cgagaacggc cggagccccc tcccgcccaa |
| 5461 | gcagaagcgg aagatggagc ggacgatcga gcccgaggtg GCTACTAACT TCAGCCTGCT |
| 5521 | GAAGCAGGCT GGCGACGTGG AGGAGAACCC TGGACCTATG GTGAGCAAGG GCGAGGAGGA |
| 5581 | TAACATGGCC ATCATCAAGG AGTTCATGCG CTTCAAGGTG CACATGGAGG GCTCCGTGAA |
| 5641 | CGGCCACGAG TTCGAGATCG AGGGCGAGGG CGAGGGCCGC CCCTACGAGG GCACCCAGAC |
| 5701 | CGCCAAGCTG AAGGTGACCA AGGGTGGtCC CCTGCCCTTC GCCTGGGACA TCCTGTCCCC |
| 5761 | TCAGTTCATG TACGGCTCCA AGGCCTACGT GAAGCACCCC GCCGACATCC CCGACTACTT |
| 5821 | GAAGCTGTCC TTCCCCGAGG GCTTCAAGTG GGAGCGCGTG ATGAACTTCG AGGACGGCGG |
| 5881 | CGTGGTGACC GTGACCCAGG ACTCCTCCCT GCAGGACGGC GAGTTCATCT ACAAGGTGAA |
| 5941 | GCTGCGCGGC ACCAACTTCC CCTCCGACGG CCCCGTAATG CAGAAGAAGA CCATGGGCTG |
| 6001 | GGAGGCCTCC TCCGAGCGGA TGTACCCCGA GGACGGCGCC CTGAAGGGCG AGATCAAGCA |
| 6061 | GAGGCTGAAG CTGAAGGACG GCGGCCACTA CGACGCTGAG GTCAAGACCA CCTACAAGGC |
| 6121 | CAAGAAGCCC GTGCAGCTGC CCGGCGCCTA CAACGTCAAC ATCAAGTTGG ACATCACCTC |
| 6181 | CCACAACGAG GACTACACCA TCGTGGAACA GTACGAACGC GCCGAGGGCC GCCACTCCAC |
| 6241 | CGGCGGCATG GACGAGCTGT ACAAGTGATA ATATGTTACG TGCAAAGGTG ATTGTCACCC |
| 6301 | CCCGAAAGAC CATATTGTGA CACACCCTCA GTATCACGCC CAAACATTTA CAGCCGCGGT |
| 6361 | GTCAAAAACC GCGTGGACGT GGTTAACATC CCTGCTGGGA GGATCAGCCG TAATTATTAT |
| 6421 | AATTGGCTTG GTGCTGGCTA CTATTGTGGC CATGTACGTG CTGACCAACC AGAAACATAA |
| 6481 | TTGAATACAG CAGCAATTGG CAAGCTGCTT ACATAGAACT CGCGGCGATT GGCATGCCGC |
| 6541 | CTTAAAATTT TTATTTTATT TTTCTTTTCT TTTCCGAATC GGATTTTGTT TTTAATATTT |
| 6601 | CAAAAAAAAA AAAAAAAAAA AAAAAAAAAA AAAAAAAAAA ATAGGGATAA CAGGGTAATT |
| 6661 | GAGCAAAAGG CCAGCAAAAG GCCAGGAACC GTAAAAAGGC CGCGTTGCTG GCGTTTTTCC |
| 6721 | ATAGGCTCCG CCCCCCTGAC GAGCATCACA AAAATCGACG CTCAAGTCAG AGGTGGCGAA |
| 6781 | ACCCGACAGG ACTATAAAGA TACCAGGCGT TTCCCCCTGG AAGCTCCCTC GTGCGCTCTC |
| 6841 | CTGTTCCGAC CCTGCCGCTT ACCGGATACC TGTCCGCCTT TCTCCCTTCG GGAAGCGTGG |
| 6901 | CGCTTTCTCA TAGCTCACGC TGTAGGTATC TCAGTTCGGT GTAGGTCGTT CGCTCCAAGC |
| 6961 | TGGGCTGTGT GCACGAACCC CCCGTTCAGC CCGACCGCTG CGCCTTATCC GGTAACTATC |
| 7021 | GTCTTGAGTC CAACCCGGTA AGACACGACT TATCGCCACT GGCAGCAGCC ACTGGTAACA |
| 7081 | GGATTAGCAG AGCGAGGTAT GTAGGCGGTG CTACAGAGTT CTTGAAGTGG TGGCCTAACT |
| 7141 | ACGGCTACAC TAGAAGAACA GTATTTGGTA TCTGCGCTCT GCTGAAGCCA GTTACCTTCG |
| 7201 | GAAAAAGAGT TGGTAGCTCT TGATCCGGCA AACAAACCAC CGCTGGTAGC GGTGGTTTTT |
| 7261 | TTGTTTGCAA GCAGCAGATT ACGCGCAGAA AAAAAGGATC TCAAGAAGAT CCTTTGATCT |
| 7321 | TTTCTACGGG GTCTGACGCT CAGTGGAACG AAAACTCACG TTAAGGGATT TTGGTCATGA |
| 7381 | GATTATCAAA AAGGATCTTC ACCTAGATCC TTTTAAATTA AAAATGAAGT TTTAAATCAA |
| 7441 | TCTAAAGTAT ATATGAGTAA ACTTGGTCTG ACAGTTACCA ATGCTTAATC AGTGAGGCAC |
| 7501 | CTATCTCAGC GATCTGTCTA TTTCGTTCAT CCATAGTTGC CTGACTCCCC GTCGTGTAGA |
| 7561 | TAACTACGAT ACGGGAGGGC TTACCATCTG GCCCCAGTGC TGCAATGATA CCGCGAGACC |
| 7621 | CACGCTCACC GGCTCCAGAT TTATCAGCAA TAAACCAGCC AGCCGGAAGG GCCGAGCGCA |
| 7681 | GAAGTGGTCC TGCAACTTTA TCCGCCTCCA TCCAGTCTAT TAATTGTTGC CGGGAAGCTA |
| 7741 | GAGTAAGTAG TTCGCCAGTT AATAGTTTGC GCAACGTTGT TGCCATTGCT ACAGGCATCG |
| 7801 | TGGTGTCACG CTCGTCGTTT GGTATGGCTT CATTCAGCTC CGGTTCCCAA CGATCAAGGC |
| 7861 | GAGTTACATG ATCCCCCATG TTGTGCAAAA AAGCGGTTAG CTCCTTCGGT CCTCCGATCG |
| 7921 | TTGTCAGAAG TAAGTTGGCC GCAGTGTTAT CACTCATGGT TATGGCAGCA CTGCATAATT |
| 7981 | CTCTTACTGT CATGCCATCC GTAAGATGCT TTTCTGTGAC TGGTGAGTAC TCAACCAAGT |
| 8041 | CATTCTGAGA ATAGTGTATG CGGCGACCGA GTTGCTCTTG CCCGGCGTCA ATACGGGATA |
| 8101 | ATACCGCGCC ACATAGCAGA ACTTTAAAAG TGCTCATCAT TGGAAAACGT TCTTCGGGGC |
| 8161 | GAAAACTCTC AAGGATCTTA CCGCTGTTGA GATCCAGTTC GATGTAACCC ACTCGTGCAC |
| 8221 | CCAACTGATC TTCAGCATCT TTTACTTTCA CCAGCGTTTC TGGGTGAGCA AAAACAGGAA |
| 8281 | GGCAAAATGC CGCAAAAAAG GGAATAAGGG CGACACGGAA ATGTTGAATA CTCATACTCT |
| 8341 | TCCTTTTTCA ATATTATTGA AGCATTTATC AGGGTTATTG TCTCATGAGC GGATACATAT |
| 8401 | TTGAATGTAT TTAGAAAAAT AAACAAATAG GGGTTCCGCG CACATTTCCC CGAAAAGTGC |
| 8461 | CACCTGACGT TAGGGATAAC AGGGTAATTA ATACGACTCA CTATAATGGG CGGCGCATGA |
| 8521 | GAGAAGCCCA GACCAATTAC CTACCCAAAA TGGAGAAAGT TCACGTTGAC ATCGAGGAAG |
| 8581 | ACAGCCCATT CCTCAGAGCT TTGCAGCGGA GCTTCCCGCA GTTTGAGGTA GAAGCCAAGC |
| 8641 | AGGTCACTGA TAATGACCAT GCTAATGCCA GAGCGTTTTC GCATCTGGCT TCAAAACTGA |
| 8701 | TCGAAACGGA GGTGGACCCA TCCGACACGA TCCTTGACAT TGGAAGTGCG CCCGCCCGCA |
| 8761 | GAATGTATTC TAAGCACAAG TATCATTGTA TCTGTCCGAT GAGATGTGCG GAAGATCCGG |
| 8821 | ACAGATTGTA TAAGTATGCA ACTAAGCTGA AGAAAAACTG TAAGGAAATA ACTGATAAGG |
| 8881 | AATTGGACAA GAAAATGAAG GAGCTCGCCG CCGTCATGAG CGACCCTGAC CTGGAAACTG |
| 8941 | AGACTATGTG CCTCCACGAC GACGAGTCGT GTCGCTACGA AGGGCAAGTC GCTGTTTACC |
| 9001 | AGGATGTATA CGCGGTTGAC GGACCGACAA GTCTCTATCA CCAAGCCAAT AAGGGAGTTA |
| 9061 | GAGTCGCCTA CTGGATAGGC TTTGACACCA CCCCTTTTAT GTTTAAGAAC TTGGCTGGAG |
| 9121 | CATATCCATC ATACTCTACC AACTGGGCCG ACGAAACCGT GTTAACGGCT CGTAACATAG |
| 9181 | GCCTATGCAG CTCTGACGTT ATGGAGCGGT CACGTAGAGG GATGTCCATT CTTAGAAAGA |
| 9241 | AGTATTTGAA ACCATCCAAC AATGTTCTAT TCTCTGTTGG CTCGACCATC TACCACGAGA |
| 9301 | AGAGGGACTT ACTGAGGAGC TGGCACCTGC CGTCTGTATT TCACTTACGT GGCAAGCAAA |
| 9361 | ATTACACATG TCGGTGTGAG ACTATAGTTA GTTGCGACGG GTACGTCGTT AAAAGAATAG |
| 9421 | CTATCAGTCC AGGCCTGTAT GGGAAGCCTT CAGGCTATGC TGCTACGATG CACCGCGAGG |
| 9481 | GATTCTTGTG CTGCAAAGTG ACAGACACAT TGAACGGGGA GAGGGTCTCT TTTCCCGTGT |
| 9541 | GCACGTATGT GCCAGCTACA TTGTGTGACC AAATGACTGG CATACTGGCA ACAGATGTCA |
| 9601 | GTGCGGACGA CGCGCAAAAA CTGCTGGTTG GGCTCAACCA GCGTATAGTC GTCAACGGTC |
| 9661 | GCACCCAGAG AAACACCAAT ACCATGAAAA ATTACCTTTT GCCCGTAGTG GCCCAGGCAT |
| 9721 | TTGCTAGGTG GGCAAAGGAA TATAAGGAAG ATCAAGAAGA TGAAAGGCCA CTAGGACTAC |
| 9781 | GAGATAGACA GTTAGTCATG GGGTGTTGTT GGGCTTTTAG AAGGCACAAG ATAACATCTA |
| 9841 | TTTATAAGCG CCCGGATACC CAAACCATCA TCAAAGTGAA CAGCGATTTC CACTCATTCG |
| 9901 | TGCTGCCCAG GATAGGCAGT AACACATTGG AGATCGGGCT GAGAACAAGA ATCAGGAAAA |
| 9961 | TGTTAGAGGA GCACAAGGAG CCGTCACCTC TCATTACCGC CGAGGACGTA CAAGAAGCTA |
| 10021 | AGTGCGCAGC CGATGAGGCT AAGGAGGTGC GTGAAGCCGA GGAGTTGCGC GCAGCTCTAC |
| 10081 | CACCTTTGGC AGCTGATGTT GAGGAGCCCA CTCTGGAAGC CGATGTCGAC TTGATGTTAC |
| 10141 | AAGAGGCTGG GGCCGGCTCA GTGGAGACAC CTCGTGGCTT GATAAAGGTT ACCAGCTACG |
| 10201 | ATGGCGAGGA CAAGATCGGC TCTTACGCTG TGCTTTCTCC GCAGGCTGTA CTCAAGAGTG |
| 10261 | AAAAATTATC TTGCATCCAC CCTCTCGCTG AACAAGTCAT AGTGATAACA CACTCTGGCC |
| 10321 | GAAAAGGGCG TTATGCCGTG GAACCATACC ATGGTAAAGT AGTGGTGCCA GAGGGACATG |
| 10381 | CAATACCCGT CCAGGACTTT CAAGCTCTGA GTGAAAGTGC CACCATTGTG TACAACGAAC |
| 10441 | GTGAGTTCGT AAACAGGTAC CTGCACCATA TTGCCACACA TGGAGGAGCG CTGAACACTG |
| 10501 | ATGAAGAATA TTACAAAACT GTCAAGCCCA GCGAGCACGA CGGCGAATAC CTGTACGACA |
| 10561 | TCGACAGGAA ACAGTGCGTC AAGAAAGAAC TAGTCACTGG GCTAGGGCTC ACAGGCGAGC |
| 10621 | TGGTGGATCC TCCCTTCCAT GAATTCGCCT ACGAGAGTCT GAGAACACGA CCAGCCGCTC |
| 10681 | CTTACCAAGT ACCAACCATA GGGGTGTATG GCGTGCCAGG ATCAGGCAAG TCTGGCATCA |
| 10741 | TTAAAAGCGC AGTCACCAAA AAAGATCTAG TGGTGAGCGC CAAGAAAGAA AACTGTGCAG |
| 10801 | AAATTATAAG GGACGTCAAG AAAATGAAAG GGCTGGACGT CAATGCCAGA ACTGTGGACT |
| 10861 | CAGTGCTCTT GAATGGATGC AAACACCCCG TAGAGACCCT GTATATTGAC GAAGCTTTTG |
| 10921 | CTTGTCATGC AGGTACTCTC AGAGCGCTCA TAGCCATTAT AAGACCTAAA AAGGCAGTGC |
| 10981 | TCTGCGGGGA TCCCAAACAG TGCGGTTTTT TTAACATGAT GTGCCTGAAA GTGCATTTTA |
| 11041 | ACCACGAGAT TTGCACACAA GTCTTCCACA AAAGCATCTC TCGCCGTTGC ACTAAATCTG |
| 11101 | TGACTTCGGT CGTCTCAACC TTGTTTTACG ACAAAAAAAT GAGAACGACG AATCCGAAAG |
| 11161 | AGACTAAGAT TGTGATTGAC ACTACCGGCA GTACCAAACC TAAGCAGGAC GATCTCATTC |
| 11221 | TCACTTGTTT CAGAGGGTGG GTGAAGCAGT TGCAAATAGA TT |

Non-cytopathic-NS1-EGFP (SEQ ID NO: 8)

| | |
|---|---|
| 1 | ATGTGCACCC TCATATCATG TGGTGCGAGG GGATATTGCC ACGGCCACCG AAGGAGTGAT |
| 61 | TATAAATGCT GCTAACAGCA AAGGACAACC TGGCGGAGGG GTGTGCGGAG CGCTGTATAA |
| 121 | GAAATTCCCG GAAAGCTTCG ATTTACAGCC GATCGAAGTA GGAAAAGCGC GACTGGTCAA |
| 181 | AGGTGCAGCT AAACATATCA TTCATGCCGT AGGACCAAAC TTCAACAAAG TTTCGGAGGT |
| 241 | TGAAGGTGAC AAACAGTTGG CAGAGGCTTA TGAGTCCATC GCTAAGATTG TCAACGATAA |
| 301 | CAATTACAAG TCAGTAGCGA TTCCACTGTT GTCCACCGGC ATCTTTTCCG GGAACAAAGA |
| 361 | TCGACTAACC CAATCATTGA ACCATTTGCT GACAGCTTTA GACACCACTG ATGCAGATGT |
| 421 | AGCCATATAC TGCAGGGACA AGAAATGGGA AATGACTCTC AAGGAAGCAG TGGCTAGGAG |
| 481 | AGAAGCAGTG GAGGAGATAT GCATATCCGA CGACTCTTCA GTGACAGAAC CTGATGCAGA |
| 541 | GCTGGTGAGG GTGCATCCGA AGAGTTCTTT GGCTGGAAGG AAGGGCTACA GCACAAGCGA |
| 601 | TGGCAAAACT TTCTCATATT TGGAAGGGAC CAAGTTTCAC CAGGCGGCCA AGGATATAGC |
| 661 | AGAAATTAAT GCCATGTGGC CCGTTGCAAC GGAGGCCAAT GAGCAGGTAT GCATGTATAT |
| 721 | CCTCGGAGAA AGCATGAGCA GTATTAGGTC GAAATGCCCC GTCGAAGAGT CGGAAGCCTC |
| 781 | CACACCACCT AGCACGCTGC CTTGCTTGTG CATCCATGCC ATGACTCCAG AAAGAGTACA |
| 841 | GCGCCTAAAA GCCTCACGTC CAGAACAAAT TACTGTGTGC TCATCCTTTC CATTGCCGAA |
| 901 | GTATAGAATC ACTGGTGTGC AGAAGATCCA ATGCTCCCAG CCTATATTGT TCTCACCGAA |
| 961 | AGTGCCTGCG TATATTCATC CAAGGAAGTA TCTCGTGGAA ACACCACCGG TAGACGAGAC |
| 1021 | TCCGGAGCCA TCGGCAGAGA ACCAATCCAC AGAGGGGACA CCTGAACAAC CACCACTTAT |
| 1081 | AACCGAGGAT GAGACCAGGA CTAGAACGCC TGAGCCGATC ATCATCGAAG AGGAAGAAGA |
| 1141 | GGATAGCATA AGTTTGCTGT CAGATGGCCC GACCCACCAG GTGCTGCAAG TCGAGGCAGA |
| 1201 | CATTCACGGG CCGCCCTCTG TATCTAGCTC ATCCTGGTCC ATTCCTCATG CATCCGACTT |
| 1261 | TGATGTGGAC AGTTTATCCA TACTTGACAC CCTGGAGGGA GCTAGCGTGA CCAGCGGGGC |
| 1321 | AACGTCAGCC GAGACTAACT CTTACTTCGC AAAGAGTATG GAGTTTCTGG CGCGACCGGT |
| 1381 | GCCTGCGCCT CGAACAGTAT TCAGGAACCC TCCACATCCC GCTCCGCGCA CAAGAACACC |
| 1441 | GTCACTTGCA CCCAGCAGGG CCTGCTCGAG AACCAGCCTA GTTTCCACCC CGCCAGGCGT |
| 1501 | GAATAGGGTG ATCACTAGAG AGGAGCTCGA GGCGCTTACC CCGTCACGCA CTCCTAGCAG |
| 1561 | GTCGGTCTCG AGAACCAGCC TGGTCTCCAA CCCGCCAGGC GTAAATAGGG TGATTACAAG |
| 1621 | AGAGGAGTTT GAGGCGTTCG TAGCACAACA ACAATGACGG TTTGATGCGG GTGCATACAT |
| 1681 | CTTTTCCTCC GACACCGGTC AAGGGCATTT ACAACAAAAA TCAGTAAGGC AAACGGTGCT |
| 1741 | ATCCGAAGTG GTGTTGGAGA GGACCGAATT GGAGATTTCG TATGCCCCGC GCCTCGACCA |
| 1801 | AGAAAAAGAA GAATTACTAC GCAAGAAATT ACAGTTAAAT CCCACACCTG CTAACAGAAG |
| 1861 | CAGATACCAG TCCAGGAAGG TGGAGAACAT GAAAGCCATA ACAGCTAGAC GTATTCTGCA |
| 1921 | AGGCCTAGGG CATTATTTGA AGGCAGAAGG AAAAGTGGAG TGCTACCGAA CCCTGCATCC |
| 1981 | TGTTCCTTTG TATTCATCTA GTGTGAACCG TGCCTTTTCA AGCCCCAAGG TCGCAGTGGA |
| 2041 | AGCCTGTAAC GCCATGTTGA AAGAGAACTT TCCGACTGTG GCTTCTTACT GTATTATTCC |
| 2101 | AGAGTACGAT GCCTATTTGG ACATGGTTGA CGGAGCTTCA TGCTGCTTAG ACACTGCCAG |
| 2161 | TTTTTGCCCT GCAAAGCTGC GCAGCTTTCC AAAGAAACAC TCCTATTTGG AACCCACAAT |
| 2221 | ACGATCGGCA GTGCCTTCAG CGATCCAGAA CACGCTCCAG AACGTCCTGG CAGCTGCCAC |
| 2281 | AAAAAGAAAT TGCAATGTCA CGCAAATGAG AGAATTGCCC GTATTGGATT CGGCGGCCTT |
| 2341 | TAATGTGGAA TGCTTCAAGA AATATGCGTG TAATAATGAA TATTGGGAAA CGTTTAAAGA |
| 2401 | AAACCCCATC AGGCTTACTG AAGAAAACGT GGTAAATTAC ATTACCAAAT TAAAAGGACC |
| 2461 | AAAAGCTGCT GCTCTTTTTG CGAAGACACA TAATTTGAAT ATGTTGCAGG ACATACCAAT |
| 2521 | GGACAGGTTT GTAATGGACT TAAAGAGAGA CGTGAAAGTG ACTCCAGGAA CAAAACATAC |
| 2581 | TGAAGAACGG CCCAAGGTAC AGGTGATCCA GGCTGCCGAT CCGCTAGCAA CAGCGTATCT |
| 2641 | GTGCGGAATC CACCGAGAGC TGGTTAGGAG ATTAAATGCG GTCCTGCTTC CGAACATTCA |
| 2701 | TACACTGTTT GATATGTCGG CTGAAGACTT TGACGCTATT ATAGCCGAGC ACTTCCAGCC |
| 2761 | TGGGGATTGT GTTCTGGAAA CTGACATCGC GTCGTTTGAT AAAAGTGAGG ACGACGCCAT |
| 2821 | GGCTCTGACC GCGTTAATGA TTCTGGAAGA CTTAGGTGTG GACGCAGAGC TGTTGACGCT |
| 2881 | GATTGAGGCG GCTTTCGGCG AAATTTCATC AATACATTTG CCCACTAAAA CTAAATTTAA |
| 2941 | ATTCGGAGCC ATGATGAAAT CTGGAATGTT CCTCACACTG TTTGTGAACA CAGTCATTAA |
| 3001 | CATTGTAATC GCAAGCAGAG TGTTGAGAGA ACGGCTAACC GGATCACCAT GTGCAGCATT |
| 3061 | CATTGGAGAT GACAATATCG TGAAAGGAGT CAAATCGGAC AAATTAATGG CAGACAGGTG |
| 3121 | CGCCACCTGG TTGAATATGG AAGTCAAGAT TATAGATGCT GTGGTGGGCG AGAAAGCGCC |
| 3181 | TTATTTCTGT GGAGGGTTTA TTTTGTGTGA CTCCGTGACC GGCACAGCGT GCCGTGTGGC |
| 3241 | AGACCCCCTA AAAAGGCTGT TTAAGCTTGG CAAACCTCTG GCAGCAGACG ATGAACATGA |
| 3301 | TGATGACAGG AGAAGGGCAT TGCATGAAGA GTCAACACGC TGGAACCGAG TGGGTATTCT |
| 3361 | TTCAGAGCTG TGCAAGGCAG TAGAATCAAG GTATGAAACC GTAGGAACTT CCATCATAGT |
| 3421 | TATGGCCATG ACTACTCTAG CTAGCAGTGT TAAATCATTC AGCTACCTGA GAGGGGCCCC |
| 3481 | TATAACTCTC TACGGCTAAC CTGAATGGAC TACGACATAG TCTAGTCCGC CAAGGCCACC |
| 3541 | atggacagca acacggtgtc ctccttccag gtggactgct tcctctggca cgtgcgcaag |
| 3601 | cgcttcgccg accaggagct gggcgacgcc cccttcctgg accgccttcg ccgggaccag |
| 3661 | aagtccctgc ggggccgggg cagcacgctt ggcctggaca tccgcacggc cacccgggag |
| 3721 | gggaagcaca tcgtggagcg gatcctggag gaggagtcgg acgaggccct gaagatgacg |
| 3781 | atcgcgagcg tgcccgcgcc ccggtaccta accgagatga cgctggagga gatgagcagg |
| 3841 | gactggctga tgctcatccc caagcagaag gtgaccgggt ccctctgcat acgcatggac |
| 3901 | caggccatca tggacaagga catcatcctg aaggccaact tcagcgtcat ctttaaccgg |
| 3961 | ctggaggccc tcatcctgct ccgcgccttc accgacgagg gggccattgt gggggagatc |
| 4021 | agccccctcc ccagcctgcc gggccacacc gaggaggacg tcaagaacgc catcggggto |
| 4081 | ctcatcggcg gcctcgagtg gaacgacaac accgtccgcg tgagcgagac cctccagcgg |
| 4141 | ttcacgtggc gcagctctga cgagaacggc cggagccccc tcccgcccaa gcagaagcgg |
| 4201 | aagatggagc ggacgatcga gcccgaggtg GCTACTAACT TCAGCCTGCT GAAGCAGGCT |
| 4261 | GGCGACGTGG AGGAGAACCC TGGACCTatg gtgagcaagg gcgaggagct gttcaccggg |
| 4321 | gtggtgccca tcctggtcga gctggacggc gacgtaaacg gccacaagtt cagcgtgtcc |
| 4381 | ggcgagggcg agggcgatgc cacctacggc aagctgaccc tgaagttcat ctgcaccacc |
| 4441 | ggcaagctgc ccgtgccctg gcccaccctc gtgaccaccc tgacctacgg cgtgcagtgc |
| 4501 | ttcagccgct accccgacca catgaagcag cacgacttct tcaagtccgc catgcccgaa |
| 4561 | ggctacgtcc aggagcgcac catcttcttc aaggacgacg gcaactacaa gacccgcgcc |
| 4621 | gaggtgaagt tcgagggcga caccctggtg aaccgcatcg agctgaaggg catcgacttc |
| 4681 | aaggaggacg gcaacatcct ggggcacaag ctggagtaca actacaacag ccacaacgtc |
| 4741 | tatatcatgg ccgacaagca gaagaacggc atcaaggtga acttcaagat ccgccacaac |
| 4801 | atcgaggacg gcagcgtgca gctcgccgac cactaccagc agaacacccc catcggcgac |
| 4861 | ggccccgtgc tgctgcccga caaccactac ctgagcaccc agtccgccct gagcaaagac |
| 4921 | cccaacgaga agcgcgatca catggtcctg ctggagttcg tgaccgccgc cgggatcact |
| 4981 | ctcggcatgg acgagctgta caagtaaTGA TAATATGTTA CGTGCAAAGG TGATTGTCAC |
| 5041 | CCCCCGAAAG ACCATATTGT GACACACCCT CAGTATCACG CCCAAACATT TACAGCCGCG |
| 5101 | GTGTCAAAAA CCGCGTGGAC GTGGTTAACA TCCCTGCTGG GAGGATCAGC CGTAATTATT |
| 5161 | ATAATTGGCT TGGTGCTGGC TACTATTGTG GCCATGTACG TGCTGACCAA CCAGAAACAT |
| 5221 | AATTGAATAC AGCAGCAATT GGCAAGCTGC TTACATAGAA CTCGCGGCGA TTGGCATGCC |
| 5281 | GCCTTAAAAT TTTTATTTTA TTTTTCTTTT CTTTTCCGAA TCGGATTTTG TTTTTAATAT |
| 5341 | TTCAAAAAAA AAAAAAAAAA AAAAAAAAAA AAAAAAAAAA AAAAaaagaa gagcGCAGCT |
| 5401 | CTGGCCCGTG TCTCAAAATC TCTGATGTTA CATTGCACAA GATAAAAATA TATCATCATG |
| 5461 | AACAATAAAA CTGTCTGCTT ACATAAACAG TAATACAAGG GGTGTTATGA GCCATATTCA |
| 5521 | ACGGGAAACG TCGAGGCCGC GATTAAATTC CAACATGGAT GCTGATTTAT ATGGGTATAA |
| 5581 | ATGGGCTCGC GATAATGTCG GGCAATCAGG TGCGACAATC TATCGCTTGT ATGGGAAGCC |
| 5641 | CGATGCGCCA GAGTTGTTTC TGAAACATGG CAAAGGTAGC GTTGCCAATG ATGTTACAGA |
| 5701 | TGAGATGGTC AGACTAAACT GGCTGACGGA ATTTATGCCT CTTCCGACCA TCAAGCATTT |
| 5761 | TATCCGTACT CCTGATGATG CATGGTTACT CACCACTGCG ATCCCCGGAA AAACAGCATT |
| 5821 | CCAGGTATTA GAAGAATATC CTGATTCAGG TGAAAATATT GTTGATGCGC TGGCAGTGTT |
| 5881 | CCTGCGCCGG TTGCATTCGA TTCCTGTTTG TAATTGTCCT TTTAACAGCG ATCGCGTATT |
| 5941 | TCGTCTCGCT CAGGCGCAAT CACGAATGAA TAACGGTTTG GTTGATGCGA GTGATTTTGA |
| 6001 | TGACGAGCGT AATGGCTGGC CTGTTGAACA AGTCTGGAAA GAAATGCATA AACTTTTGCC |
| 6061 | ATTCTCACCG GATTCAGTCG TCACTCATGG TGATTTCTCA CTTGATAACC TTATTTTTGA |
| 6121 | CGAGGGGAAA TTAATAGGTT GTATTGATGT TGGACGAGTC GGAATCGCAG ACCGATACCA |
| 6181 | GGATCTTGCC ATCCTATGGA ACTGCCTCGG TGAGTTTTCT CCTTCATTAC AGAAACGGCT |
| 6241 | TTTTCAAAAA TATGGTATTG ATAATCCTGA TATGAATAAA TTGCAGTTTC ATTTGATGCT |
| 6301 | CGATGAGTTT TTCTAATCAG AATTGGTTAA TTGGTTGTAA CACTGGCAGA GCATTACGCT |
| 6361 | GACTTGACGG GACGGCGCAA GCTCATGACC AAAATCCCTT AACGTGAGTT ACGCGTCGTT |
| 6421 | CCACTGAGCG TCAGACCCCG TAGAAAAGAT CAAAGGATCT TCTTGAGATC CTTTTTTTCT |
| 6481 | GCGCGTAATC TGCTGCTTGC AAACAAAAAA ACCACCGCTA CCAGCGGTGG TTTGTTTGCC |
| 6541 | GGATCAAGAG CTACCAACTC TTTTTCCGAA GGTAACTGGC TTCAGCAGAG CGCAGATACC |
| 6601 | AAATACTGTT CTTCTAGTGT AGCCGTAGTT AGGCCACCAC TTCAAGAACT CTGTAGCACC |
| 6661 | GCCTACATAC CTCGCTCTGC TAATCCTGTT ACCAGTGGCT GCTGCCAGTG GCGATAAGTC |
| 6721 | GTGTCTTACC GGGTTGGACT CAAGACGATA GTTACCGGAT AAGGCGCAGC GGTCGGGCTG |
| 6781 | AACGGGGGGT TCGTGCACAC AGCCCAGCTT GGAGCGAACG ACCTACACCG AACTGAGATA |
| 6841 | CCTACAGCGT GAGCTATGAG AAAGCGCCAC GCTTCCCGAA GGGAGAAAGG CGGACAGGTA |
| 6901 | TCCGGTAAGC GGCAGGGTCG GAACAGGAGA GCGCACGAGG GAGCTTCCAG GGGGAAACGC |
| 6961 | CTGGTATCTT TATAGTCCTG TCGGGTTTCG CCACCTCTGA CTTGAGCGTC GATTTTTGTG |
| 7021 | ATGCTCGTCA GGGGGGCGGA GCCTATGGAA AAACGCCAGC AACGCGGCCT TTTTACGGTT |
| 7081 | CCTGGCCTTT TGCTGGCCTT TTGCTCACAT gctcttctaa gTAATACGAC TCACTATAAT |
| 7141 | GGGCGGCGCA TGAGAGAAGC CCAGACCAAT TACCTACCCA AAATGGAGAA AGTTCACGTT |
| 7201 | GACATCGAGG AAGACAGCCC ATTCCTCAGA GCTTTGCAGC GGAGCTTCCC GCAGTTTGAG |
| 7261 | GTAGAAGCCA AGCAGGTCAC TGATAATGAC CATGCTAATG CCAGAGCGTT TTCGCATCTG |
| 7321 | GCTTCAAAAC TGATCGAAAC GGAGGTGGAC CCATCCGACA CGATCCTTGA CATTGGAAGT |
| 7381 | GCGCCCGCCC GCAGAATGTA TTCTAAGCAC AAGTATCATT GTATCTGTCC GATGAGATGT |
| 7441 | GCGGAAGATC CGGACAGATT GTATAAGTAT GCAACTAAGC TGAAGAAAAA CTGTAAGGAA |
| 7501 | ATAACTGATA AGGAATTGGA CAAGAAAATG AAGGAGCTCG CCGCCGTCAT GAGCGACCCT |
| 7561 | GACCTGGAAA CTGAGACTAT GTGCCTCCAC GACGACGAGT CGTGTCGCTA CGAAGGGCAA |
| 7621 | GTCGCTGTTT ACCAGGATGT ATACGCGGTT GACGGACCGA CAAGTCTCTA TCACCAAGCC |
| 7681 | AATAAGGGAG TTAGAGTCGC CTACTGGATA GGCTTTGACA CCACCCCTTT TATGTTTAAG |
| 7741 | AACTTGGCTG GAGCATATCC ATCATACTCT ACCAACTGGG CCGACGAAAC CGTGTTAACG |
| 7801 | GCTCGTAACA TAGGCCTATG CAGCTCTGAC GTTATGGAGC GGTCACGTAG AGGGATGTCC |
| 7861 | ATTCTTAGAA AGAAGTATTT GAAACCATCC AACAATGTTC TATTCTCTGT TGGCTCGACC |
| 7921 | ATCTACCACG AGAAGAGGGA CTTACTGAGG AGCTGGCACC TGCCGTCTGT ATTTCACTTA |
| 7981 | CGTGGCAAGC AAAATTACAC ATGTCGGTGT GAGACTATAG TTAGTTGCGA CGGGTACGTC |
| 8041 | GTTAAAAGAA TAGCTATCAG TCCAGGCCTG TATGGGAAGC CTTCAGGCTA TGCTGCTACG |
| 8101 | ATGCACCGCG AGGGATTCTT GTGCTGCAAA GTGACAGACA CATTGAACGG GGAGAGGGTC |
| 8161 | TCTTTTCCCG TGTGCACGTA TGTGCCAGCT ACATTGTGTG ACCAAATGAC TGGCATACTG |
| 8221 | GCAACAGATG TCAGTGCGGA CGACGCGCAA AAACTGCTGG TTGGGCTCAA CCAGCGTATA |
| 8281 | GTCGTCAACG GTCGCACCCA GAGAAACACC AATACCATGA AAAATTACCT TTTGCCCGTA |
| 8341 | GTGGCCCAGG CATTTGCTAG GTGGGCAAAG GAATATAAGG AAGATCAAGA AGATGAAAGG |
| 8401 | CCACTAGGAC TACGAGATAG ACAGTTAGTC ATGGGGTGTT GTTGGGCTTT TAGAAGGCAC |
| 8461 | AAGATAACAT CTATTTATAA GCGCCCGGAT ACCCAAACCA TCATCAAAGT GAACAGCGAT |
| 8521 | TTCCACTCAT TCGTGCTGCC CAGGATAGGC AGTAACACAT TGGAGATCGG GCTGAGAACA |
| 8581 | AGAATCAGGA AAATGTTAGA GGAGCACAAG GAGCCGTCAC CTCTCATTAC CGCCGAGGAC |
| 8641 | GTACAAGAAG CTAAGTGCGC AGCCGATGAG GCTAAGGAGG TGCGTGAAGC CGAGGAGTTG |
| 8701 | CGCGCAGCTC TACCACCTTT GGCAGCTGAT GTTGAGGAGC CCACTCTGGA AGCCGATGTC |
| 8761 | GACTTGATGT TACAAGAGGC TGGGGCCGGC TCAGTGGAGA CACCTCGTGG CTTGATAAAG |
| 8821 | GTTACCAGCT ACGATGGCGA GGACAAGATC GGCTCTTACG CTGTGCTTTC TCCGCAGGCT |
| 8881 | GTACTCAAGA GTGAAAAATT ATCTTGCATC CACCCTCTCG CTGAACAAGT CATAGTGATA |
| 8941 | ACACACTCTG GCCGAAAAGG GCGTTATGCC GTGGAACCAT ACCATGGTAA AGTAGTGGTG |
| 9001 | CCAGAGGGAC ATGCAATACC CGTCCAGGAC TTTCAAGCTC TGAGTGAAAG TGCCACCATT |
| 9061 | GTGTACAACG AACGTGAGTT CGTAAACAGG TACCTGCACC ATATTGCCAC ACATGGAGGA |
| 9121 | GCGCTGAACA CTGATGAAGA ATATTACAAA ACTGTCAAGC CCAGCGAGCA CGACGGCGAA |
| 9181 | TACCTGTACG ACATCGACAG GAAACAGTGC GTCAAGAAAG AACTAGTCAC TGGGCTAGGG |
| 9241 | CTCACAGGCG AGCTGGTGGA TCCTCCCTTC CATGAATTCG CCTACGAGAG TCTGAGAACA |
| 9301 | CGACCAGCCG CTCCTTACCA AGTACCAACC ATAGGGGTGT ATGGCGTGCC AGGATCAGGC |
| 9361 | AAGTCTGGCA TCATTAAAAG CGCAGTCACC AAAAAAGATC TAGTGGTGAG CGCCAAGAAA |
| 9421 | GAAAACTGTG CAGAAATTAT AAGGGACGTC AAGAAAATGA AAGGGCTGGA CGTCAATGCC |
| 9481 | AGAACTGTGG ACTCAGTGCT CTTGAATGGA TGCAAACACC CCGTAGAGAC CCTGTATATT |
| 9541 | GACGAAGCTT TTGCTTGTCA TGCAGGTACT CTCAGAGCGC TCATAGCCAT TATAAGACCT |
| 9601 | AAAAAGGCAG TGCTCTGCGG GGATCCCAAA CAGTGCGGTT TTTTTAACAT GATGTGCCTG |
| 9661 | AAAGTGCATT TTAACCACGA GATTTGCACA CAAGTCTTCC ACAAAAGCAT CTCTCGCCGT |
| 9721 | TGCACTAAAT CTGTGACTTC GGTCGTCTCA ACCTTGTTTT ACGACAAAAA AATGAGAACG |
| 9781 | ACGAATCCGA AAGAGACTAA GATTGTGATT GACACTACCG GCAGTACCAA ACCTAAGCAG |
| 9841 | GACGATCTCA TTCTCACTTG TTTCAGAGGG TGGGTGAAGC AGTTGCAAAT AGATTACAAA |
| 9901 | GGCAACGAAA TAATGACGGC AGCTGCCTCT CAAGGGCTGA CCCGTAAAGG TGTGTATGCC |
| 9961 | GTTCGGTACA AGGTGAATGA AAATCCTCTG TACGCACCCA CCTCAGAACA TGTGAACGTC |
| 10021 | CTACTGACCC GCACGGAGGA CCGCATCGTG TGGAAAACAC TAGCCGGCGA CCCATGGATA |
| 10081 | AAAACACTGA CTGCCAAGTA CCCTGGGAAT TTCACTGCCA CGATAGAGGA GTGGCAAGCA |
| 10141 | GAGCATGATG CCATCATGAG GCACATCTTG GAGAGACCGG ACCCTACCGA CGTCTTCCAG |
| 10201 | AATAAGGCAA ACGTGTGTTG GGCCAAGGCT TTAGTGCCGG TGCTGAAGAC CGCTGGCATA |
| 10261 | GACATGACCA CTGAACAATG GAACACTGTG GATTATTTTG AAACGGACAA AGCTCACTCA |
| 10321 | GCAGAGATAG TATTGAACCA ACTATGCGTG AGGTTCTTTG GACTCGATCT GGACTCCGGT |
| 10381 | CTATTTTCTG CACCCACTGT TCCGTTATCC ATTAGGAATA ATCACTGGGA TAACTCCCCG |
| 10441 | TCGCCTAACA TGTACGGGCT GAATAAAGAA GTGGTCCGTC AGCTCTCTCG CAGGTACCCA |
| 10501 | CAACTGCCTC GGGCAGTTGC CACTGGAAGA GTCTATGACA TGAACACTGG TACACTGCGC |
| 10561 | AATTATGATC CGCGCATAAA CCTAGTACCT GTAAACAGAA GACTGCCTCA TGCTTTAGTC |
| 10621 | CTCCACCATA ATGAACACCC ACAGAGTGAC TTTTCTTCAT TCGTCAGCAA ATTGAAGGGC |
| 10681 | AGAACTGTCC TGGTGGTCGG GGAAAAGTTG TCCGTCCCAG GCAAAATGGT TGACTGGTTG |
| 10741 | TCAGACCGGC CTGAGGCTAC CTTCAGAGCT CGGCTGGATT TAGGCATCCC AGGTGATGTG |
| 10801 | CCCAAATATG ACATAATATT TGTTAATGTG AGGACCCCAT ATAAATACCA TCACTATCAG |
| 10861 | CAGTGTGAAG ACCATGCCAT TAAGCTTAGC ATGTTGACCA AGAAAGCTTG TCTGCATCTG |
| 10921 | AATCCCGGCG GAACCTGTGT CAGCATAGGT TATGGTTACG CTGACAGGGC CAGCGAAAGC |
| 10981 | ATCATTGGTG CTATAGCGCG GCTGTTCAAG TTTTCCCGGG TATGCAAACC GAAATCCTCA |
| 11041 | CTTGAAGAGA CGGAAGTTCT GTTTGTATTC ATTGGGTACG ATCGCAAGGC CCGTACGCAC |
| 11101 | AATCCTTACA AGCTTTCATC AACCTTGACC AACATTTATA CAGGTTCCAG ACTCCACGAA |
| 11161 | GCCGG |

What is claimed is:

1. A polynucleotide or a set of polynucleotides comprising a first nucleic acid molecule encoding an influenza non-structural (NS1) protein and a second nucleic acid molecule encoding a heterologous target mRNA.

2. The polynucleotide or set of polynucleotides of claim 1, wherein the first nucleic acid molecule encoding the influenza NS1 protein and the second nucleic acid molecule encoding the target mRNA are present in a first vector.

3. The polynucleotide or set of polynucleotides of claim 1, wherein the first nucleic acid molecule encoding the influenza NS1 protein is present in a first vector, and wherein the second nucleic acid molecule encoding the target mRNA is present in a second vector.

4. The polynucleotide or set of polynucleotides of any one of claims 1 to 3, wherein the first nucleic acid molecule encoding the influenza NS1 protein is expressed under the control of a first promoter.

5. The polynucleotide or set of polynucleotides of any one of claims 1 to 4, wherein the second nucleic acid molecule encoding the target mRNA is expressed under the control of a second promoter.

6. The polynucleotide or set of polynucleotides of claim 5, wherein the first promoter and the second promoter are the same.

7. The polynucleotide or set of polynucleotides of claim 5, wherein the first promoter and the second promoter are the different.

8. The polynucleotide or set of polynucleotides of claim 1 or 2, wherein the first nucleic acid molecule encoding the influenza NS1 protein and the second nucleic acid molecule encoding the target mRNA are expressed under the control of a first promoter, wherein the first promoter drives expression of both the influenza NS1 protein and the target mRNA.

9. The polynucleotide or set of polynucleotides of claim 8, wherein the first nucleic acid molecule encoding the influenza NS1 protein and the second nucleic acid molecule encoding the target mRNA are linked by an IRES sequence.

10. The polynucleotide or set of polynucleotides of any one of claims 2 to 9, wherein the first vector, the second vector, or both comprise one or more regulatory elements.

11. The polynucleotide or set of polynucleotides of any one of claims 1 to 10, wherein the expression of the target mRNA is increased relative to the expression of the target mRNA in the absence of the first nucleic acid molecule encoding the influenza NS1 protein.

12. The polynucleotide or set of polynucleotides of claim 11, wherein the expression of the target mRNA is increased by at least about 10%, at least about 20%, at least about 25%, at least about 30%, at least about 40%, at least about 50%, at least about 60%, at least about 70%, at least about 75%, at least about 80%, at least about 90%, at least about 100%, at least about 125%, at least about 150%, at least about 175%, at least about 200%, at least about 225%, at least about 250%, at least about 275%, or at least about 300% relative to the expression of the target mRNA in the absence of the first nucleic acid molecule encoding the influenza NS1 protein.

13. The polynucleotide or set of polynucleotides of claim 11 or 12, wherein the increase in the expression of the target mRNA persists for at least about 6 hours, at least about 12 hours, at least about 18 hours, at least about 24 hours, at least about 30 hours, at least about 36 hours, at least about 42 hours, or at least about 48 hours.

14. The polynucleotide or set of polynucleotides of any one of claims 1 to 13, wherein the target mRNA encodes a biologically active polypeptide.

15. The polynucleotide or set of polynucleotides of claim 14, wherein the biologically active polypeptide comprises a cytokine, a chemokine, a growth factor, a clotting factor, an enzyme, or any combination thereof.

16. The polynucleotide or set of polynucleotides of claim 15, wherein the cytokine is IL-1α, IL-1β, IL-1RA, IL-18, IL-2, IL-4, IL-7, IL-9, IL-13, IL-15, IL-3, IL-5, GM-CSF, IL-6, IL-11, G-CSF, IL,-12, LIF, OSM, IL,-10, IL,-20, IL,-14, IL,-16, IL,-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP, a fragment thereof, a variant thereof, or any combination thereof.

17. The polynucleotide or set of polynucleotides of any one of claims 1 to 15, wherein the target mRNA encodes an IL-12 polypeptide or a fragment or variant thereof.

18. The polynucleotide or set of polynucleotides of claim 17, wherein the target mRNA encodes a p35 subunit of IL-12 and a p40 subunit of IL-12.

19. The polynucleotide or set of polynucleotides of claim 18, wherein the p35 subunit and the p40 subunit are expressed from a single promoter.

20. The polynucleotide or set of polynucleotides of claim 18 or 19, wherein the p35 subunit and the p40 subunit are expressed as a single contiguous polypeptide.

21. The polynucleotide or set of polynucleotides of any one of claims 18 to 20, wherein the p35 subunit and the p40 subunit are linked by one or more covalent bonds.

22. The polynucleotide or set of polynucleotides of any one of claims 18 to 21, wherein the p35 subunit and the p40 subunit are linked by one or more peptide bonds.

23. The polynucleotide or set of polynucleotides of claim 18 or 19, wherein a portion of the mRNA that encodes the p35 subunit is separated from a portion of the mRNA that encodes the p40 subunit by an IRES.

24. The polynucleotide or set of polynucleotides of any one of claims 1 to 13, wherein the target mRNA encodes a miRNA, siRNA, shRNA, a dsRNA, antisense oligonucleotide, a guide RNA, or any combination thereof.

25. The polynucleotide or set of polynucleotides of any one of claims 4 to 24, wherein the first promoter is an inducible promoter, a tissue specific promoter, or a constitutively active promoter.

26. The polynucleotide or set of polynucleotides of any one of claims 5 to 25, wherein the second promoter is an inducible promoter, a tissue specific promoter, or a constitutively active promoter.

27. The polynucleotide or set of polynucleotides of any one of claims 1 to 26, wherein the influenza NS1 is a type A influenza virus NS1, a type B influenza virus NS1, a type C influenza virus NS1, or a variant thereof.

28. The polynucleotide or set of polynucleotides of any one of claims 1 to 26, wherein the influenza NS1 is an H1N1 NS1, H1N2 NS1, H2N2 NS1, H3N2 NS1, H5N1 NS1, H7N9 NS1, H7N7 NS1, H9N2 NS1, H7N2 NS1, H7N3 NS1, H5N2 NS1, H10N7 NS1, or any combination thereof.

29. The polynucleotide or set of polynucleotides of any one of claims 1 to 28, wherein the influenza NS1 is H5N1 NS1.

30. The polynucleotide or set of polynucleotides of any one of claims 1 to 28, wherein the influenza NS1 is H1N1 NS1.

31. The polynucleotide or set of polynucleotides of claim 30, wherein the H1N1 NS1 is the H1N1 TX91 variant NS1.

32. The polynucleotide or set of polynucleotides of any one of claims 1 to 31, wherein the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 1 or 2.

33. The polynucleotide or set of polynucleotides of any one of claims 1 to 32, wherein the influenza NS1 encoded by the first nucleic acid molecule comprises the amino acid sequence encoded by the nucleotide sequence set forth in SEQ ID NO: 1 or 2.

34. The polynucleotide or set of polynucleotides of any one of claims 1 to 33, wherein the first nucleic acid molecule comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1 or 2, wherein the nucleotide sequence encodes an influenza NS1 protein.

35. The polynucleotide or set of polynucleotides of any one of claims 1 to 32, wherein the first nucleic acid molecule comprises the nucleotide sequence set forth in SEQ ID NO: 1 or 2, wherein the nucleotide sequence encodes an influenza NS1 protein.

36. The polynucleotide or set of polynucleotides of any one of claims 1 to 35, comprising one or more modified nucleic acid molecule.

37. The polynucleotide or set of polynucleotides of any one of claims 1 to 36, wherein: (i) the first nucleic acid molecule, (ii) the second nucleic acid molecule, or (iii) both (i) and (ii) are circular RNA.

38. A polynucleotide or a set of polynucleotides comprising a self-replicating target mRNA, wherein the self-replicating target mRNA comprises one or more modified nucleic acid molecule.

39. The polynucleotide or set of polynucleotides of any one of claims 1 to 38, wherein less than about 50%, less than about 45%, less than about 40%, less than about 35%, less than about 30%, or less than about 25% of the nucleic acids in the polynucleotide or the set of polynucleotides are modified nucleic acid molecules.

40. The polynucleotide or set of polynucleotides of any one of claims 1 to 39, wherein about 25% of the nucleic acids in the polynucleotide or the set of polynucleotides are modified nucleic acid molecules.

41. The polynucleotide or set of polynucleotides of any one of claims 36 to 40, wherein the one or more modified nucleic acid molecule is a modified rNTP.

42. The polynucleotide or the set of polynucleotides of any one of claims 36 to 41, wherein the one or more modified nucleic acid molecule comprises N1-methylpsuedo uracil, 5-methyl cytosine, N6-methyladenosine, or combinations thereof.

43. A vector or a set of vectors comprising the polynucleotide or the set of polynucleotides of any one of claims 1 to 42.

44. The vector or the set of vectors of claim 43, which is a replicon.

45. The vector or the set of vectors of claim 43 or 44, which is a Venezuelan equine encephalitis (VEE) replicon or a derivative or portion thereof.

46. The vector or the set of vectors of claim any one of claims 43 to 45, which is a Venezuelan equine encephalitis (VEE) replicon comprising a nucleotide sequence encoding a lysine at residue 739, according to the wild-type amino acid sequence VEE.

47. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA; and (v) a VEE 3′UTR or a derivative thereof.

48. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA; and (vi) a VEE 3′UTR or a derivative thereof.

49. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA; (v) an E1 sequence; and (vi) a VEE 3′UTR or a derivative thereof.

50. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA; (vi) an E1 sequence;
and (vii) a VEE 3′UTR or a derivative thereof.

51. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a second nucleic acid molecule encoding the target mRNA, wherein the target mRNA encodes a human IL-12 polypeptide; and (v) a VEE 3′UTR or a derivative thereof.

52. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding the influenza NS1 protein; (iv) a P2A linker; (v) a second nucleic acid molecule encoding the target mRNA, wherein the target mRNA encodes a human IL-12 polypeptide; (vi) an E1 sequence; and (vii) a VEE 3′UTR or a derivative thereof.

53. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a second nucleic acid molecule encoding the target mRNA; and (v) a VEE 3′UTR or a derivative thereof.

54. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a P2A linker; (v) a second nucleic acid molecule encoding the target mRNA; (vi) an E1 sequence; and (vii) a VEE 3′UTR or a derivative thereof.

55. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a second nucleic acid molecule encoding the target mRNA, wherein the target mRNA encodes a human IL-12 polypeptide; and (v) a VEE 3′UTR or a derivative thereof.

56. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H1N1 TX91 variant NS1 or an H5N1 NS1; (iv) a P2A linker; (v) a second nucleic acid molecule encoding the target mRNA, wherein the target mRNA encodes a human IL-12 polypeptide; (vi) an E1 sequence; and
(vii) a VEE 3′UTR or a derivative thereof.

57. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H1N1 TX91 variant NS1; (iv) a second nucleic acid molecule encoding the target mRNA, wherein the target mRNA encodes a human IL-12 polypeptide; and (v) a VEE 3′UTR or a derivative thereof.

58. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1; (iv) a second nucleic acid molecule encoding the target mRNA, wherein the target mRNA encodes a human IL-12 polypeptide; and (v) a VEE 3′UTR or a derivative thereof.

59. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule comprising the nucleotide sequence set forth in SEQ ID NO: 1; (iv) a second nucleic acid molecule encoding the target mRNA, wherein the target mRNA encodes a human IL-12 polypeptide; and (v) a VEE 3′UTR or a derivative thereof.

60. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule encoding an H5N1 NS1; (iv) a P2A linker; (v) a second nucleic acid molecule encoding the target mRNA, wherein the target mRNA encodes a human IL-12 polypeptide; (vi) an E1 sequence; and (vii) a VEE 3′UTR or a derivative thereof.

61. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule comprising a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 2; (iv) a second nucleic acid molecule encoding the target mRNA, wherein the target mRNA encodes a human IL-12 polypeptide; and (v) a VEE 3′UTR or a derivative thereof.

62. The vector or the set of vectors of claim any one of claims 43 to 46, wherein the vector comprises (i) a VEE 5′UTR or a derivative thereof; (ii) one or more non-structural protein (nsP); (iii) a first nucleic acid molecule comprising the nucleotide sequence set forth in SEQ ID NO: 2; (iv) a second nucleic acid molecule encoding the target mRNA, wherein the target mRNA encodes a human IL-12 polypeptide; and (v) a 3′UTR from a parent replicon, e.g., a 3′UTR from VEE.

63. The vector of the set of vectors of any one of claims 47 to 62, wherein the one or more nsP comprises a VEE nsP or a derivative thereof.

64. The vector of the set of vectors of claim 63, wherein the VEE nsP is selected from nsP2, nsP3, nsP4, and any combination thereof.

65. A cell comprising the polynucleotide or set of polynucleotides of any one of claims 1 to 42 or the vector or the set of vectors of any one of claims 43 to 64.

66. The cell of claim 65, which is a mammalian cell.

67. The cell of claim 65 or 66, which is a human cell.

68. The cell of any one of claims 65 to 67, which is an immune cell.

69. A pharmaceutical composition comprising the polynucleotide or set of polynucleotides of any one of claims 1 to 42, the vector or the set of vectors of any one of claims 43 to 64, or the cell of any one of claims 65 to 68 and a pharmaceutically acceptable carrier.

70. A method of expressing a target mRNA in a cell, comprising transfecting the cell with the polynucleotide or set of polynucleotides of any one of claims 1 to 42 or the vector or the set of vectors of any one of claims 43 to 64.

71. The method of claim 70, wherein the cell is a human cell.

72. The method of claim 70 or 71, wherein the cell is an ex vivo human cell.

73. The method of any one of claims 70 to 72, wherein the cell is a human immune cell.

74. A method of treating a subject in need thereof, comprising administering to the subject the polynucleotide or set of polynucleotides of any one of claims 1 to 42, the vector or the set of vectors of any one of claims 43 to 64, the cell of any one of claims 65 to 68, or the pharmaceutical composition of claim 69.

75. A method of expressing a target mRNA in a subject in need thereof, comprising administering to the subject the polynucleotide or set of polynucleotides of any one of claims 1 to 42, the vector or the set of vectors of any one of claims 43 to 64, the cell of any one of claims 66 to 68, or the pharmaceutical composition of claim 69.

76. The method of claim 74 or 75, wherein the subject is afflicted with a cancer.

77. The method of claim 76, wherein the cancer is selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, Hodgkin's lymphoma, and any combination thereof.

78. A method of expressing a target mRNA in a cell, comprising co-expressing the target mRNA and an influenza NS1 protein in the cell, wherein the target mRNA is not an influenza mRNA.

79. The method of claim 78, wherein the influenza NS1 protein is encoded by a first nucleic acid molecule and the target mRNA is encoded by a second nucleic acid molecule.

80. The method of claim 78 or 79, wherein the first nucleic acid molecule encoding the influenza NS1 protein and the second nucleic acid molecule encoding the target mRNA are present in a first vector.

81. The method of claim 78 or 79, wherein the first nucleic acid molecule encoding the influenza NS1 protein is present in a first vector, and wherein the second nucleic acid molecule encoding the target mRNA is present in a second vector.

82. The method of any one of claims 78 to 81, wherein the first nucleic acid molecule encoding the influenza NS1 protein is expressed under the control of a first promoter.

83. The method of any one of claims 78 to 82, wherein the second nucleic acid molecule encoding the target mRNA is expressed under the control of a second promoter.

84. The method of claim 83, wherein the first promoter and the second promoter are the same.

85. The method of claim 83, wherein the first promoter and the second promoter are the different.

86. The method of any one of claims 78 to 81, wherein the first nucleic acid molecule encoding the influenza NS1 protein and the second nucleic acid molecule encoding the target mRNA are expressed under the control of a first promoter, wherein the first promoter drives expression of both the influenza NS1 protein and the target mRNA.

87. The method of claim 86, wherein the first nucleic acid molecule encoding the influenza NS1 protein and the second nucleic acid molecule encoding the target mRNA are linked by an IRES sequence.

88. The method of any one of claims 80 to 87, wherein the first vector, the second vector, or both comprise one or more regulatory elements.

89. The method of any one of claims 78 to 88, wherein expression of the target mRNA is increased relative to the expression of the target mRNA in the absence of the first nucleic acid molecule encoding the influenza NS1 protein.

90. The method of any one of claims 79 to 89, wherein: (i) the first nucleic acid molecule, (ii) the second nucleic acid molecule, or (iii) both (i) and (ii) are circular RNA.

91. A method of expressing a target mRNA in a cell, comprising transfecting the cell with a polynucleotide or a set of polynucleotides comprising a self-replicating target mRNA comprising one or more modified nucleic acid molecules.

92. The of claim 91, wherein less than about 50%, less than about 45%, less than about 40%, less than about 35%, less than about 30%, or less than about 25% of the nucleic acids in the polynucleotide or the set of polynucleotides are modified nucleic acid molecules.

93. The method of claims 91 or 92, wherein about 25% of the nucleic acids in the polynucleotide or the set of polynucleotides are modified nucleic acid molecules.

94. The method any one of claims 91 to 93, wherein the one or more modified nucleic acid molecules is a modified rNTP.

95. The method of any one of claims 91 to 94, wherein the one or more modified nucleic acid molecules comprises N1-methylpsuedo uracil, 5-methyl cytosine, N6-methyladenosine, or combinations thereof.

96. The method of any one of claims 91 to 95, wherein expression of the target mRNA is increased relative to the expression of the target mRNA from a self-replicating target mRNA not comprising one or more modified nucleic acid molecules.

97. The method of claim 96, wherein the expression of the target mRNA is increased by at least about 10%, at least about 20%, at least about 25%, at least about 30%, at least about 40%, at least about 50%, at least about 60%, at least about 70%, at least about 75%, at least about 80%, at least about 90%, at least about 100%, at least about 125%, at least about 150%, at least about 175%, at least about 200%, at least about 225%, at least about 250%, at least about 275%, or at least about 300%.

98. The method of claim 96 or 97, wherein the increase in the expression of the target mRNA persists for at least about 6 hours, at least about 12 hours, at least about 18 hours, at least about 24 hours, at least about 30 hours, at least about 36 hours, at least about 42 hours, or at least about 48 hours.

99. The method of any one of claims 70 to 98, wherein the target mRNA encodes a biologically active polypeptide.

100. The method of claim 99, wherein the biologically active polypeptide comprises a cytokine, a chemokine, a growth factor, a clotting factor, an enzyme, or any combination thereof.

101. The method of claim 100, wherein the cytokine is IL-1α, IL-1β, IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP, a fragment thereof, a variant thereof, or any combination thereof.

102. The method of claim 101, wherein the target mRNA encodes an IL-12 polypeptide or a fragment or variant thereof.

103. The method of claim 102, wherein the target mRNA encodes a p35 subunit of IL-12 and a p40 subunit of IL-12.

104. The method of claim 103, wherein the p35 subunit and the p40 subunit are expressed from a single promoter.

105. The method of claim 103 or 104, wherein the p35 subunit and the p40 subunit are expressed as a single contiguous polypeptide.

106. The method of any one of claims 103 to 105, wherein the p35 subunit and the p40 subunit are linked by one or more covalent bonds.

107. The method of any one of claims 103 to 106, wherein the p35 subunit and the p40 subunit are linked by one or more peptide bonds.

108. The method of claim 103 or 107, wherein a portion of the mRNA that encodes the p35 subunit is separated from a portion of the mRNA that encodes the p40 subunit by an IRES.

109. The method of any one of claims 70 to 98, wherein the target mRNA encodes a miRNA, siRNA, shRNA, a dsRNA, antisense oligonucleotide, a guide RNA, or any combination thereof.

110. The method of any one of claims 82 to 90, wherein the first promoter is an inducible promoter, a tissue specific promoter, or a constitutively active promoter.

111. The method of any one of claims 83 to 90, 109, and 110, wherein the second promoter is an inducible promoter, a tissue specific promoter, or a constitutively active promoter.

112. The method of any one of claims 78 to 90 and 109 to 111, wherein the influenza NS1 is a type A influenza virus NS1, a type B influenza virus NS1, a type C influenza virus NS1, or a variant thereof.

113. The method of any one of claims 78 to 90 and 109 to 112, wherein the influenza NS1 is an H1N1 NS1, H1N2 NS1, H2N2 NS1, H3N2 NS1, H5N1 NS1, H7N9 NS1, H7N7 NS1, H9N2 NS1, H7N2 NS1, H7N3 NS1, H5N2 NS1, H10N7 NS1, or a combination thereof.

114. The method of any one of claims 78 to 90 and 109 to 113, wherein the influenza NS1 is H5N1 NS1.

115. The method of any one of claims 78 to 90 and 109 to 113, wherein the influenza NS1 is H1N1 NS1.

116. The method of claim 115, wherein the H1N1 NS1 is the H1N1 TX91 variant NS1.

117. The method of any one of claims 78 to 90 and 109 to 113, wherein the influenza NS1 encoded by the first nucleic acid molecule comprises an amino acid sequence having at least about 80%, at least about 85%, at least about 90%, at least about 95%, at least about 96%, at least about 97%, at least about 98%, or at least about 99% sequence identity to the amino acid sequence encoded by the nucleotide sequence set forth in SEQ ID NO: 1 or 2.

118. The method of any one of claims 78 to 90 and 109 to 113, wherein the influenza NS1 encoded by the first nucleic acid molecule comprises the amino acid sequence encoded by the nucleotide sequence set forth in SEQ ID NO: 1 or 2.

119. The method of any one of claims 78 to 90 and 109 to 113, wherein the first nucleic acid molecule comprises a nucleotide sequence having at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 1 or 2, wherein the nucleotide sequence encodes an influenza NS1 protein.

120. The method of any one of claims 78 to 90 and 109 to 113, wherein the first nucleic acid molecule comprises the nucleotide sequence set forth in SEQ ID NO: 1 or 2, wherein the nucleotide sequence encodes an influenza NS1 protein.


SEQUENCE LISTING (8 sequences)

SEQ ID NO: 1 - DNA, 10497 bp, synthetic construct
  misc_feature 1..10497: note = Influenza H1N1 TX91 variant NS1
  source 1..10497: mol_type = other DNA
agattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggacagcaacacggtgtcaagcttccaggtcgactgcttcctgtggcacgtgcgcaagcaggtggcggaccaggagctgggcgacgccccgttcctcgaccggctgcgccgcgaccagaagtccctgaaggggcggggcagcacgctcggcctgaacatcgagaccgcgacgtgcgtcggcaagcagatcgtggagcggatcctcaaggaggagagcgacgaggccttccgcatgaccatggcgagcgccctggcgtcgcgctacctcacggacatgacgatcgaggagatgagccgggactggttcatgctcatgcccaagcagaaggtggcggggcccctctgcgtgcggatggaccaggccatcatggacaagaacatcatcctgaaggcgaacttcagcgtgatcttcgaccgcctggagacgctgaccctcctgcgcgcattcaccgaggagggggcgatcgtcggcgagatcagcccgctgccgtccctgccggggcacacgaacgaggacgtcaagaacgccatcggcgtcctcatcgggggcctcgagtggaacgacaacaccgtccgggtctccgagacgctccagcggttcgcgtggcggagcagcaacgagaacggcgggccgccgctcacccccacgcagaagcggaagatggccggaaagatccgctccgaggtctgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaatagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgttagggataacagggtaattaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaat

SEQ ID NO: 2 - DNA, 10497 bp, synthetic construct
  misc_feature 1..10497: note = Influenza H5N1 NS1
  source 1..10497: mol_type = other DNA
tagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggacagcaacacggtgtcctccttccaggtggactgcttcctctggcacgtgcgcaagcgcttcgccgaccaggagctgggcgacgcccccttcctggaccgccttcgccgggaccagaagtccctgcggggccggggcagcacgcttggcctggacatccgcacggccacccgggaggggaagcacatcgtggagcggatcctggaggaggagtcggacgaggccctgaagatgacgatcgcgagcgtgcccgcgccccggtacctaaccgagatgacgctggaggagatgagcagggactggctgatgctcatccccaagcagaaggtgaccgggtccctctgcatacgcatggaccaggccatcatggacaaggacatcatcctgaaggccaacttcagcgtcatctttaaccggctggaggccctcatcctgctccgcgccttcaccgacgagggggccattgtgggggagatcagccccctccccagcctgccgggccacaccgaggaggacgtcaagaacgccatcggggtcctcatcggcggcctcgagtggaacgacaacaccgtccgcgtgagcgagaccctccagcggttcacgtggcgcagctctgacgagaacggccggagccccctcccgcccaagcagaagcggaagatggagcggacgatcgagcccgaggtgtgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaatagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgttagggataacagggtaattaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaa

SEQ ID NO: 3 - DNA, 10418 bp, synthetic construct
  misc_feature 1..10418: note = Strand-EGFP
  source 1..10418: mol_type = other DNA
gctcttctaagtaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaagaagagcgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 4 - DNA, 10516 bp, synthetic construct
  misc_feature 1..10516: note = Strand-mCherry
  source 1..10516: mol_type = other DNA
cgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgttagggataacagggtaattaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggtcccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaatagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataacta

SEQ ID NO: 5 - DNA, 10418 bp, synthetic construct
  misc_feature 1..10418: note = Non-cytopathic-EGFP
  source 1..10418: mol_type = other DNA
gctcttctaagtaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggctgttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaagaagagcgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 6 - DNA, 11165 bp, synthetic construct
  misc_feature 1..11165: note = Non-cytopathic-EGFP
  source 1..11165: mol_type = other DNA
gctcttctaagtaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggacagcaacacggtgtcctccttccaggtggactgcttcctctggcacgtgcgcaagcgcttcgccgaccaggagctgggcgacgcccccttcctggaccgccttcgccgggaccagaagtccctgcggggccggggcagcacgcttggcctggacatccgcacggccacccgggaggggaagcacatcgtggagcggatcctggaggaggagtcggacgaggccctgaagatgacgatcgcgagcgtgcccgcgccccggtacctaaccgagatgacgctggaggagatgagcagggactggctgatgctcatccccaagcagaaggtgaccgggtccctctgcatacgcatggaccaggccatcatggacaaggacatcatcctgaaggccaacttcagcgtcatctttaaccggctggaggccctcatcctgctccgcgccttcaccgacgagggggccattgtgggggagatcagccccctccccagcctgccgggccacaccgaggaggacgtcaagaacgccatcggggtcctcatcggcggcctcgagtggaacgacaacaccgtccgcgtgagcgagaccctccagcggttcacgtggcgcagctctgacgagaacggccggagccccctcccgcccaagcagaagcggaagatggagcggacgatcgagcccgaggtggctactaacttcagcctgctgaagcaggctggcgacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaagaagagcgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 7 - DNA, 11262 bp, synthetic construct
  misc_feature 1..11262: note = Strand NS1-mCherry
  source 1..11262: mol_type = other DNA
acaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggacagcaacacggtgtcctccttccaggtggactgcttcctctggcacgtgcgcaagcgcttcgccgaccaggagctgggcgacgcccccttcctggaccgccttcgccgggaccagaagtccctgcggggccggggcagcacgcttggcctggacatccgcacggccacccgggaggggaagcacatcgtggagcggatcctggaggaggagtcggacgaggccctgaagatgacgatcgcgagcgtgcccgcgccccggtacctaaccgagatgacgctggaggagatgagcagggactggctgatgctcatccccaagcagaaggtgaccgggtccctctgcatacgcatggaccaggccatcatggacaaggacatcatcctgaaggccaacttcagcgtcatctttaaccggctggaggccctcatcctgctccgcgccttcaccgacgagggggccattgtgggggagatcagccccctccccagcctgccgggccacaccgaggaggacgtcaagaacgccatcggggtcctcatcggcggcctcgagtggaacgacaacaccgtccgcgtgagcgagaccctccagcggttcacgtggcgcagctctgacgagaacggccggagccccctcccgcccaagcagaagcggaagatggagcggacgatcgagcccgaggtggctactaacttcagcctgctgaagcaggctggcgacgtggaggagaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggtcccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaatagggataacagggtaattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgttagggataacagggtaattaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagatt

SEQ ID NO: 8 - DNA, 11165 bp, synthetic construct
  misc_feature 1..11165: note = Non-cytopathic-NS1-EGFP
  source 1..11165: mol_type = other DNA
atgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggccaccatggacagcaacacggtgtcctccttccaggtggactgcttcctctggcacgtgcgcaagcgcttcgccgaccaggagctgggcgacgcccccttcctggaccgccttcgccgggaccagaagtccctgcggggccggggcagcacgcttggcctggacatccgcacggccacccgggaggggaagcacatcgtggagcggatcctggaggaggagtcggacgaggccctgaagatgacgatcgcgagcgtgcccgcgccccggtacctaaccgagatgacgctggaggagatgagcagggactggctgatgctcatccccaagcagaaggtgaccgggtccctctgcatacgcatggaccaggccatcatggacaaggacatcatcctgaaggccaacttcagcgtcatctttaaccggctggaggccctcatcctgctccgcgccttcaccgacgagggggccattgtgggggagatcagccccctccccagcctgccgggccacaccgaggaggacgtcaagaacgccatcggggtcctcatcggcggcctcgagtggaacgacaacaccgtccgcgtgagcgagaccctccagcggttcacgtggcgcagctctgacgagaacggccggagccccctcccgcccaagcagaagcggaagatggagcggacgatcgagcccgaggtggctactaacttcagcctgctgaagcaggctggcgacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaagaagagcgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctcttctaagtaatacgactcactataatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggctgttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccgg